(19) United States
(12) Patent Application Publication
(10) Pub. No.: US 2026/0176228 A1
(43) Pub. Date: Jun. 25, 2026

(54) MODULATORS OF LIVER RECEPTOR HOMOLOGUE 1 (LRH-1) AND USES

(71) Applicant: Emory University, Atlanta, GA (US)

(72) Inventors: Nathan Jui, Decatur, GA (US); Eric Ortlund, Decatur, GA (US); Autumn Flynn, Decatur, GA (US); Henry Zecca, Decatur, GA (US); Racheal Spurlin, Decatur, GA (US); Michael Lee Cato, Anderson, SC (US)

(73) Assignee: Emory University, Atlanta, GA (US)

(21) Appl. No.: 19/123,163

(22) PCT Filed: Oct. 27, 2023
(86) PCT No.: PCT/US2023/036120

§ 371 Date: Apr. 22, 2025

Related U.S. Application Data
(60) Provisional application No. 63/420,005, filed on Oct. 27, 2022.

(51) Int. Cl. C07C 59/72; A61K 31/192; A61P 3/10; C07C 307/08

(52) U.S. Cl. CPC C07C 59/72; A61K 31/192; A61P 3/10; C07C 307/08; C07C 2602/22

(57) ABSTRACT

This disclosure relates to modulators of liver receptor homologue 1 (LRH-1) and methods of managing disease and conditions related thereto. In certain examples, modulators are derivatives of hexahydropental ene. In certain examples, this disclosure relates to methods of treating or preventing cancer, diabetes, or cardiovascular disease by administering an effective amount of a hexahydropentalene derivative disclosed herein.

### CROSS-REFERENCE TO RELATED APPLICATION

The application claims the benefit of and the priority to U.S. Provisional Application No. 63/420,005, filed Oct. 27, 2022, which is hereby incorporated by reference in its entirety for all purposes.

### STATEMENT REGARDING FEDERALLY FUNDED RESEARCH

This invention was made with government support under Grant No. DK115213 awarded by the National Institutes of Health. The government has certain rights in the invention.

### BACKGROUND

Liver receptor homologue 1 (LRH-1) is a nuclear hormone receptor (NR) and acts as a transcription factor to control gene expression. Traditionally, LRH-1 was identified to be involved with cholesterol homeostasis and early fetal development. The use of an LRH-1 agonist for treating diabetes is reported in Lee et al. Nature, 2011, 474, 506-510. The medium-chain dietary phospholipid, dilauroyl-phosphatidylcholine (DLPC), was identified as an LRH-1 agonist. Diabetic mice fed DLPC had improved glucose tolerance and reduced hepatic fat accumulation, as well as reduced quantities of circulating insulin, triglycerides, and free fatty acids. The anti-diabetic effects were associated with changes in expression of a select subset of LRH-1 target genes involved with lipid metabolism. Importantly, the differences in health and on gene expression by DLPC were absent in LRH-1 liver-specific conditional knockout mice, directly implicating LRH-1 in these effects. In addition to DLPC, LRH-1 binds phosphatidyl-inositol 3,4,5-trisphosphate (PIP3), which is an important signaling lipid in diabetes.

LRH-1 is also aberrantly overexpressed in certain cancers. It is believed to promote tumor growth through estrogen receptor and β-catenin signaling. See Nadolny et al. Liver receptor homolog-1 (LRH-1): a potential therapeutic target for cancer. Cancer Biol Ther, 2015, 16 (7): 997-1004. Whitby et al. report small molecule agonists of LRH-1. J Med Chem 2006, 49 (23): 6652-5. See also Whitby et al., J Med Chem, 2011, 54, 2266-2281; Busby et al. Probe Reports from the NIH Molecular Libraries Program, 2010, 1:1-55; Benod et al. Antagonists of nuclear receptor LRH-1. J Biol Chem, 2013, 288:19830-44, U.S. Published Patent Application No. 2013/0210143, U.S. Published Patent Application No. 2008/0227864, and U.S. Published Patent Application No. 2004/0038862. Mays et al. report the crystal structures of LRH-1 bound to synthetic agonists. J Biol Chem, 2016, 291 (49): 25281-25291. References cited herein are not an admission of prior art.

### SUMMARY

Described herein are modulators of liver receptor homologue 1 (LRH-1) and methods of managing diseases and conditions related thereto. In certain examples, the modulators described herein are derivatives of hexahydropentalene. In certain examples, this disclosure relates to methods of treating or preventing diabetes, cancer, or cardiovascular disease by administering an effective amount of a hexahydropentalene derivative as disclosed herein to a subject in need thereof.

Described herein are compounds having the following formula:

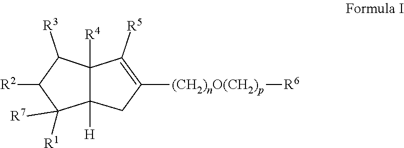

or a prodrug, salt, or stereoisomer thereof, wherein n is 1-6; p is 1-6; R¹ is alkyl, halogen, nitro, cyano, hydroxy, amino, sulfamoylamino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰; R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰; R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰; R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰; R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰, R⁶ is a hydrogen, carboxy, or alkyl wherein R⁶ is optionally terminally substituted with a hydroxy, carboxy, or phosphate, wherein the hydroxy, carboxy, or phosphate are optionally further substituted with R¹⁰; R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁷ is optionally substituted with one or more, the same or different, R¹⁰; or R¹ and R⁷ together are an oxo or oxime, wherein the oxime is optionally substituted with one or more, the same or different, R¹⁰; R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹⁰ is optionally substituted with one or more, the same or different, R¹¹; and R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

Also described herein are compounds having the following formula:

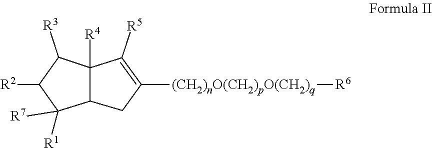

or a prodrug, salt, or stereoisomer thereof, wherein n is 1-6; p is 1-6; q is 1-6; R¹ is alkyl, halogen, nitro, cyano, hydroxy, amino, sulfamoylamino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰, R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) 2amino, alkanoyl, 1, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰; R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰; R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰; R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰; R⁶ is a hydrogen, carboxy, or alkyl wherein R⁶ is optionally terminally substituted with a hydroxy, carboxy, or phosphate, wherein the hydroxy, carboxy, or phosphate are optionally further substituted with R¹⁰; R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁷ is optionally substituted with one or more, the same or different, R¹⁰; or R¹ and R⁷ together are an oxo or oxime, wherein the oxime is optionally substituted with one or more, the same or different, R¹⁰; R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) 2amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹⁰ is optionally substituted with one or more, the same or different, R¹¹; and R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

In some cases, the compound is 5-(4-(3-phenyl-3a-(1-phenylvinyl)-6-(sulfamoylamino)-1,3a,4,5,6,6a-hexahydropentalen-2-yl) butoxy) pentanoic acid or salt thereof. In some cases, the compound is 3-(3-(2-(3-phenyl-3a-(1-phenylvinyl)-6-(sulfamoylamino)-1,3a,4,5,6,6a-hexahydropentalen-2-yl) ethoxy) propoxy) propanoic acid or salt thereof.

Additionally described herein are compounds according to the following structures:

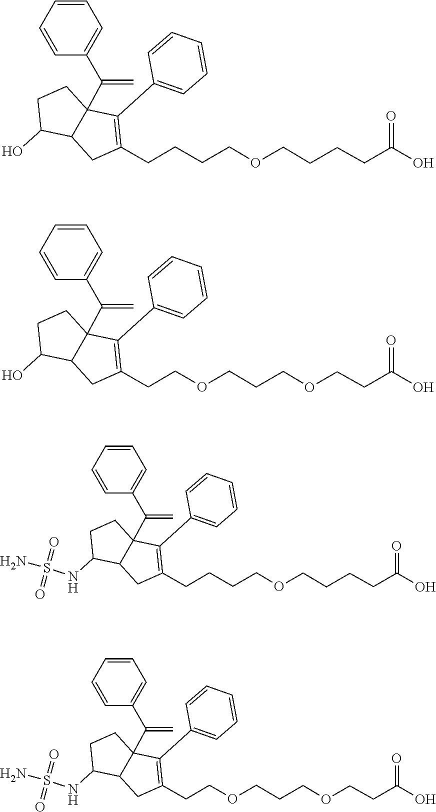 [SMILES: C=C(c1ccccc1)C12CCC(O)C1CC(CCCCOCCCCC(=O)O)=C2c1ccccc1 ; C=C(c1ccccc1)C12CCC(O)C1CC(CCOCCCOCCC(=O)O)=C2c1ccccc1 ; C=C(c1ccccc1)C12CCC(NS(N)(=O)=O)C1CC(CCCCOCCCCC(=O)O)=C2c1ccccc1 ; C=C(c1ccccc1)C12CCC(NS(N)(=O)=O)C1CC(CCOCCCOCCC(=O)O)=C2c1ccccc1]

or a prodrug or salt thereof.

In certain examples, the disclosure contemplates pharmaceutical compositions comprising compounds disclosed herein or pharmaceutically acceptable salts thereof and pharmaceutically acceptable excipients. In certain examples, the pharmaceutical products may be in the form of tablets, pills, capsules, gels, granules, or aqueous buffer solutions.

Further described herein are methods of treating or preventing cancer comprising administering an effective amount of a pharmaceutical composition as described herein to a subject in need thereof. Optionally, the cancer is selected from the group consisting of bladder cancer, breast cancer, colon cancer, rectal cancer, endometrial cancer, kidney cancer, leukemia, lung cancer, melanoma, non-Hodgkin lymphoma, pancreatic cancer, prostate cancer, and thyroid cancer.

Also described herein are methods of treating or preventing diabetes comprising administering an effective amount of a pharmaceutical composition as described herein to a subject in need thereof. Optionally, the diabetes is insulin-dependent diabetes mellitus, non insulin-dependent diabetes mellitus, or gestational diabetes.

Further described herein are methods of treating or preventing cardiovascular disease, inflammatory bowel diseases (IBD), and/or colitis or ulcerative colitis comprising administering an effective amount of a pharmaceutical composition as described herein to a subject in need thereof.

The details of one or more examples are set forth in the drawings and the description below. Other features, objects, and advantages will be apparent from the description and drawings, and from the claims.

### DESCRIPTION OF DRAWINGS

FIG. 1 shows data on pharmacokinetics, solubility, permeability, and metabolic stability of compounds disclosed herein.

FIGS. 2A, 2B, and 2C show the target gene expression driven by 6N-10CA-ME. FIG. 2A is a Volcano Plot showing RNA-seq of H-mLRH-1 mice treated with 3 mg/Kg of 6N-10CA-ME via an intraperitoneal injection. FIG. 2B is a Volcano Plot showing RNA-seq of H-mLRH-1 mice treated with 30 mg/Kg of 6N-10CA-ME via an intraperitoneal injection. FIG. 2C is a RT-qPCR showing target gene expression at 3 and 30 mg/kg doses.

FIG. 3 demonstrates that 6N-10CA-ME shows minimal cross-reactivity with other nuclear receptors (NRs). 6N-10CA-ME was introduced to reporter cells at 2 μM and the activity of respective NRs was tested. Data are normalized relative to activity induced by the appropriate reference agonist for each receptor.

FIG. 4 shows that 6N-10CA-ME reduces glucose intolerance in setting of diet-induced obesity. Male and female mice were treated with 6N-10CA-ME via an intraperitoneal injection starting at 12 weeks of High Fat Diet (HFD; 60% fat) feeding and continuing daily to 16 weeks of HFD feeding. (Panel A) Summary of body weights of mice in each group at 0, 12, and 16 weeks of HFD feeding. (Panels B and C) Fasting blood glucose (mg/dL) levels of mice in each group at 12 and 16 weeks of HFD feeding. (Panel D) Glucose tolerance tests (GTT) following administration of glucose (2 g/kg) at 12 and 16 weeks of HFD feeding. (Panel E) Area under the curve (AUC) summary of GTT at 12 and 16 weeks of HFD feeding. Data are represented as means+/−SEM. Sample size is 6-17 per group. **** p<0.0001 vs. chow. Data were analyzed with a two-way ANOVA with Tukey multiple comparison test.

### DETAILED DESCRIPTION

Described herein are modulators of liver receptor homologue 1 (LRH-1) and methods for their use in managing diseases and conditions related thereto. In certain examples, the modulators described herein are derivatives of hexahydropentalene. Also described herein are methods of treating and/or preventing diabetes, cancer, or cardiovascular disease by administering an effective amount of a hexahydropentalene derivative as disclosed herein to a subject in need thereof.

Before the present disclosure is described in greater detail, it is to be understood that this disclosure is not limited to particular examples described, and as such may, of course, vary. It is also to be understood that the terminology used herein is for the purpose of describing particular examples only, and is not intended to be limiting, since the scope of the present disclosure will be limited only by the appended claims.

### I. Definitions

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual examples described and illustrated herein has discrete components and features which may be readily separated from or combined with the features of any of the other several examples without departing from the scope or spirit of the present disclosure. Any recited method can be carried out in the order of events recited or in any other order that is logically possible.

Examples of the present disclosure will employ, unless otherwise indicated, techniques of medicine, organic chemistry, biochemistry, molecular biology, pharmacology, and the like, which are within the skill of the art. Such techniques are explained fully in the literature. It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Certain of the compounds described herein may contain one or more asymmetric centers and may give rise to enantiomers, diastereomers, and other stereoisomeric forms that can be defined, in terms of absolute stereochemistry at each asymmetric atom as (R)— or (S)—. The present chemical entities, pharmaceutical compositions and methods are meant to include all such possible isomers, including racemic mixtures, tautomer forms, hydrated forms, optically substantially pure forms, and intermediate mixtures.

Unless otherwise stated, structures depicted herein are also meant to include compounds which differ only in the presence of one or more isotopically enriched atoms. For example, compounds having the present structures except for the replacement or enrichment of a hydrogen by deuterium or tritium at one or more atoms in the molecule, or the replacement or enrichment of a carbon by ¹³C or ¹⁴C at one or more atoms in the molecule, are within the scope of this disclosure. In one example, provided herein are isotopically labeled compounds having one or more hydrogen atoms replaced by or enriched by deuterium. In one example, provided herein are isotopically labeled compounds having one or more hydrogen atoms replaced by or enriched by tritium. In one example, provided herein are isotopically labeled compounds having one or more carbon atoms replaced or enriched by ¹³C. In one example, provided herein are isotopically labeled compounds having one or more carbon atoms replaced or enriched by ¹⁴C.

The disclosure also embraces isotopically labeled compounds that are identical to those recited herein, except that one or more atoms are replaced by an atom having an atomic mass or mass number different from the atomic mass or mass number usually found in nature. Examples of isotopes that can be incorporated into disclosed compounds include isotopes of hydrogen, carbon, nitrogen, oxygen, phosphorus, sulfur, fluorine, and chlorine, such as, e.g., ²H, ³H, ¹³C, ¹⁴C, ¹⁵N, ¹⁸O, ¹⁷O, ³¹P, ³²P, ³⁵S, ¹⁸F, and ³⁶Cl, respectively. Certain isotopically labeled compounds (e.g., those labeled with ³H and/or ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium (i.e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isotopically labeled reagent. In some examples, provided herein are compounds that can also contain unnatural proportions of atomic isotopes at one or more of atoms that constitute such compounds. All isotopic variations of the compounds as disclosed herein, whether radioactive or not, are encompassed within the scope of the present disclosure.

A “linking group” refers to any variety of molecular arrangements that can be used to bridge molecular moieties together. An example formula may be —Rₘ— wherein R is selected individually and independently at each occurrence as: —CRₘRₘ—, —CHRₘ—, —CH—, —C—, —CH₂—, —C(OH) Rₘ, —C(OH) (OH)—, —C(OH) H, —C(Hal) Rₘ—, —C(Hal) (Hal)-, —C(Hal) H—, —C(N₃) Rₘ—, —C(CN) Rₘ—, —C(CN) (CN)—, —C(CN) H—, —C(N₃) (N₃)—, —C(N₃) H—, —O—, —S—, —N—, —NH—, —NRₘ—, (C═O)—, —(C═NH)—, —(C═S)—, —(C═CH₂)—, which may contain single, double, or triple bonds individually and independently between the R groups. If an R is branched with an Rₘ it may be terminated with a group such as —CH₃, —H, —CH═CH₂, —CCH, —OH, —SH, —NH₂, —N₃, —CN, or -Hal, or two branched Rs may form a cyclic structure. It is contemplated that in certain instances, the total Rs or “m” may be less than 100, 50, 25, 10, 5, 4, or 3. Examples of linking groups in include bridging alkyl groups, alkoxyalkyl groups, and polyethylene glycol.

As used herein, a “lipid” group refers to a hydrophobic group that is naturally or non-naturally occurring that is highly insoluble in water. As used herein a lipid group is considered highly insoluble in water when the point of connection on the lipid is replaced with a hydrogen and the resulting compound has a solubility of less than 3×10⁻³ w/w (at 25° C.) in water, e.g., 9.5×10⁻⁴% w/w (at 25° C.) which is the percent solubility of hexane in water by weight. See Solvent Recovery Handbook, 2^{nd} Ed, Smallwood, 2002 by Blackwell Science, page 193. Examples of naturally occurring lipids include saturated or unsaturated hydrocarbon chains found in fatty acids, glycerolipids, cholesterol, steroids, polyketides, and derivatives. Non-naturally occurring lipids include derivatives of naturally occurring lipids, acrylic polymers, and alkylated compounds and derivatives thereof.

As used herein, “alkyl” means a noncyclic straight chain or branched, unsaturated or saturated hydrocarbon such as those containing from 1 to 22 carbon atoms, while the term “lower alkyl” or “C₁₋₄ alkyl” has the same meaning as alkyl but contains from 1 to 4 carbon atoms. The term “higher alkyl” has the same meaning as alkyl but contains from 8 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or “heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic. “Arylalkyl” means an alkyl substituted with an aryl, e.g., benzyl, methyl substituted with phenyl.

As used herein, “heteroaryl” refers to an aromatic heterocarbocycle having 1 to 4 heteroatoms selected from nitrogen, oxygen, and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above attached through a sulfur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, and t-butoxy.

“Alkylamino” refers to an alkyl group as defined above attached through an amino bridge. An example of an alkylamino is methylamino, (i.e., —NH—CH₃).

“Alkanoyl” refers to an alkyl as defined above attached through a carbonyl bride (i.e., —(C═O)alkyl).

“Alkylsulfonyl” refers to an alkyl as defined above attached through a sulfonyl bridge (i.e., —S(═O)₂alkyl) such as mesyl and the like, “arylsulfonyl, sulfamoyl” refers to an aryl attached through a sulfonyl bridge (i.e., —S(═O)₂aryl).

“Aminosulfonyl” or “Sulfamoyl” refers to an amino attached through a sulfonyl bridge (i.e., —S(═O)₂NH₂).

“Sulfamoylamino” refers to a sulfamoyl attached through an amino bridge (i.e., —NH—S(═O)₂NH₂).

“Alkylsulfinyl” refers to an alkyl as defined above attached through a sulfinyl bridge (i.e. —S(═O)alkyl).

“Aminoalkyl” refers to an amino group attached through an alkyl bridge. An example of an aminoalkyl is aminomethyl, (i.e., NH₂—CH₂—).

“Hydroxyalkyl” refers to a hydroxy group attached through an alkyl bridge. An example of a hydroxyalkyl is hydroxyethyl, (i.e., HO—CH₂CH₂—).

“Hal” refers to a halogen such as F, Br, Cl, or I.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O) R_{b}, —NRₐC(═O) NRₐNR_{b}, —NRₐC(═O) OR^{b}, —NRₐSO₂R_{b}, —C(═O) Rₐ, —C(═O) ORₐ, —C(═O) NRₐR_{b}, —OC(═O) NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen, hydroxy, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

The term “optionally substituted,” as used herein, means that substitution is optional and therefore it is possible for the designated atom to be unsubstituted.

As used herein, “salts” refer to derivatives of the disclosed compounds where the parent compound is modified making acid or base salts thereof. Examples of salts include, but are not limited to, mineral salts such as sodium, potassium, or zinc carboxylic acid salts, or organic acid salts of basic residues such as amines, alkylamines, or dialkylamines; alkali or organic salts of acidic residues such as carboxylic acids; and the like. In typical examples, the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers to any animal, preferably a human patient, livestock, rodent, monkey, or domestic pet.

As used herein, the term “derivative” refers to a structurally similar compound that retains sufficient functional attributes of the identified analogue. The derivative may be structurally similar because it is lacking one or more atoms, substituted with one or more substituents, a salt, in different hydration/oxidation states, e.g., substituting a single or double bond, substituting a hydroxy group for a ketone, or because one or more atoms within the molecule are switched, such as, but not limited to, replacing an oxygen atom with a sulfur or nitrogen atom, or replacing an amino group with a hydroxy group or vice versa. Replacing a carbon with nitrogen in an aromatic ring is a contemplated derivative. The derivative may be a prodrug. Derivatives may be prepared by any variety of synthetic methods or appropriate adaptations presented in the chemical literature or as in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “prodrug” refers to an agent that is converted into a biologically active form in vivo. Prodrugs are often useful because, in some situations, they may be easier to administer than the parent compound. They may, for instance, be bioavailable by oral administration whereas the parent compound is not. The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters or enol ethers. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate, and benzoate derivatives of an alcohol or acetamide, formamide and benzamide derivatives of an amine functional group in the active compound and the like.

For example, if a disclosed compound or a pharmaceutically acceptable form of the compound contains a carboxylic acid functional group, a prodrug can comprise a pharmaceutically acceptable ester formed by the replacement of the hydrogen atom of the acid group with a group such as (C₁-C₄)alkyl, (C₁-C₅)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino (C₂-C₃)alkyl (such as beta-dimethylaminoethyl), carbamoyl-(C₁-C₂)alkyl, N,N-di-(C₁-C₂)alkylcarbamoyl-(C₁-C₂)alkyl and piperidino-, pyrrolidino- or morpholino (C₂-C₃)alkyl.

If a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆)alkanoyloxy)ethyl, 1-methyl-1 ((C₁-C₆)alkanoyloxy)ethyl (C₁-C₆)alkoxycarbonyloxymethyl, —N—(C₁-C₆)alkoxycarbonylaminomethyl, succinoyl, (C₁-C₆)alkanoyl, alpha-amino (C₁-C₄)alkanoyl, arylacyl and alpha-aminoacyl, or alpha-aminoacyl-alpha-aminoacyl, where each alpha-aminoacyl group is independently selected from naturally occurring L-amino acids P(O) (OH)₂, —P(O) (O(C₁-C₆)alkyl)₂, and glycosyl (the radical resulting from the removal of a hydroxy group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇) cycloalkyl, benzyl, a natural alpha-aminoacyl, —C(OH)C(O)OY₁ wherein Y¹ is H, (C₁-C₆)alkyl or benzyl, —C (OY₂) Y³ wherein Y² is (C₁-C₄)alkyl and Y³ is (C₁-C₆)alkyl, carboxy (C₁-C₆)alkyl, amino (C₁-C₄)alkyl or mono-Nor di-N,N—(C₁-C₆)alkylaminoalkyl, —C(Y⁴)Y₅ wherein Y⁴ is H or methyl and Y₅ is mono-N— or di-N,N—(C₁-C₆)alkylamino, morpholino, piperidin-1-yl or pyrrolidin-1-yl.

As used herein, “pharmaceutically acceptable esters” include, but are not limited to, alkyl, alkenyl, alkynyl, aryl, arylalkyl, and cycloalkyl esters of acidic groups, including, but not limited to, carboxylic acids, phosphoric acids, phosphinic acids, sulfonic acids, sulfinic acids, and boronic acids.

As used herein, “pharmaceutically acceptable enol ethers” include, but are not limited to, derivatives of formula —C═C(OR) where R can be selected from alkyl, alkenyl, alkynyl, aryl, aralkyl, and cycloalkyl. Pharmaceutically acceptable enol esters include, but are not limited to, derivatives of formula —C═C(OC(O) R) where R can be selected from hydrogen, alkyl, alkenyl, alkynyl, aryl, aralkyl, and cycloalkyl.

“Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain examples, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

A “chemotherapy agent,” “chemotherapeutic,” “anti-cancer agent” or the like, refer to molecules that are recognized to aid in the treatment of a cancer. Contemplated examples include the following molecules or derivatives such as temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, idoxifene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin and/or lenalidomide or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

As used herein, the terms “prevent” and “preventing” include the prevention of the recurrence, spread or onset. It is not intended that the present disclosure be limited to complete prevention. In some examples, the onset is delayed, or the severity of the disease is reduced.

As used herein, the terms “treat” and “treating” are not limited to the case where the subject (e.g., patient) is cured and the disease is eradicated. Rather, examples, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

Further explanations of the terms recited above are provided below in the description.

### II. Hexahydropentalene Derivatives

In certain examples, the present disclosure relates to compounds that are hexahydropentalene derivatives. The hexahydropentalene derivatives are any of the compounds disclosed herein optionally substituted with one or more substituents.

A class of compounds described herein is represented by Formula I:

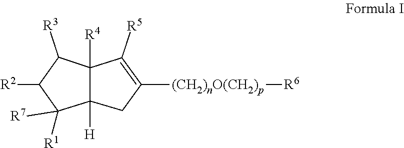

including prodrugs, salts, and stereoisomers thereof.

In Formula I, n is 1-6. For example, n can be from 2-4.

Also in Formula I, p is 1-6. For example, p can be from 2-4.

Additionally in Formula I, R¹ is alkyl, halogen, nitro, cyano, hydroxy, amino, sulfamoylamino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R¹ can be substituted with one or more, the same or different, R¹⁰ (as described below).

Further in Formula I, R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R² can be substituted with one or more, the same or different, R¹⁰.

Also in Formula I, R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R³ can be substituted with one or more, the same or different, R¹⁰;

Additionally in Formula I, R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R⁴ can be substituted with one or more, the same or different, R¹⁰.

Further in Formula I, R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R⁵ can be substituted with one or more, the same or different, R¹⁰.

Also in Formula I, R⁶ is a hydrogen, carboxy, or alkyl. In some examples, R⁶ is optionally terminally substituted with a hydroxy, carboxy, or phosphate. In some examples, R⁶ is optionally substituted with a hydroxy, carboxy, or phosphate. Optionally, the hydroxy, carboxy, or phosphate are further substituted with R¹⁰.

Additionally in Formula I, R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R⁷ is substituted with one or more, the same or different, R¹⁰.

In some cases of Formula I, R¹ and R⁷ together are an oxo or oxime. The oxime can be optionally substituted with one or more, the same or different, R¹⁰.

In Formula I, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. In some cases, R¹⁰ can be aminosulfonyl. Optionally, R¹⁰ can be optionally substituted with one or more, the same or different, R¹¹.

Also in Formula I, R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

In some examples of Formula I, n is 2, 3, or 4; p is 2, 3, or 4. In some examples, n is 2; p is 2, 3, or 4. In some examples, n is 3; p is 2, 3, or 4. In some examples, n is 4; p is 2, 3, or 4. In some examples, n is 2; p is 2. In some examples, n is 3; p is 3. In some examples, n is 4; p is 4. In some examples, n is 2; p is 3. In some examples, n is 2; p is 4. In some examples, n is 3; p is 2. In some examples, n is 3; p is 4. In some examples, n is 4; p is 2. In some examples, n is 4; p is 3.

In some examples of Formula I, R¹ is hydroxy or sulfamoylamino. In some examples, R⁷ is hydrogen. In some examples, R⁷ is hydrogen, and R¹ is hydroxy, sulfamoylamino, or sulfamic acid. In some examples, R² and R³ are hydrogen, R⁴ is 1-phenylvinyl or 1-phenylethyl, and R⁵ is phenyl and R⁷ is hydrogen. In some examples, R⁶ is alkyl terminally substituted with a hydroxy, carboxy, or phosphate, wherein the hydroxy, carboxy, or phosphate are optionally further substituted with R¹⁰. In some examples, R¹ is hydroxy, alkyl, amino, sulfamoylamino, aminoalkyl, carbamoyl, sulphate, sulfonate, aminosulfonyl, phosphate, phosphonate, or heterocyclyl, wherein R¹ is optionally substituted with R¹⁰; and R⁷ is hydrogen.

Optionally, the compounds of Formula I can be represented by Structure I-A as shown below:

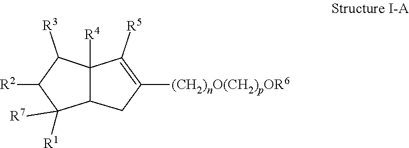

including prodrugs, salts, and stereoisomers thereof.

In Structure I-A, n, p, R¹, R², R³, R₄, R⁵, R⁷, and R¹¹, are as defined above for Formula I.

Also in Structure I-A, R⁶ is hydrogen, carboxy, or alkyl, wherein R⁶ is optionally substituted with an alkyl, hydroxy, carboxy, or phosphate, wherein the hydroxy, carboxy, or phosphate are optionally further substituted with R¹⁰.

Additionally in Structure I-A, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹ (as defined in Formula I).

In some examples of Structure I-A, n is 2, 3, or 4; p is 2, 3, or 4; and R¹ is hydroxy or sulfamoylamino. In some examples, n is 2; p is 2, 3, or 4; and R¹ is hydroxy or sulfamoylamino. In some examples, n is 3; p is 2, 3, or 4; and R¹ is hydroxy or sulfamoylamino. In some examples, n is 4; p is 2, 3, or 4; and R¹ is hydroxy or sulfamoylamino. In some examples, n is 2; p is 2; and R¹ is hydroxy or sulfamoylamino. In some examples, n is 3; p is 3; and R¹ is hydroxy or sulfamoylamino. In some examples, n is 4; p is 4; and R¹ is hydroxy or sulfamoylamino. In some examples, n is 2; p is 2; and R¹ is hydroxy or sulfamoylamino. In some examples, n is 2; p is 3; and R¹ is hydroxy or sulfamoylamino. In some examples, n is 2; p is 3; and R¹ is hydroxy or sulfamoylamino. In some examples, n is 2; p is 3; and R¹ is hydroxy or sulfamoylamino. In some examples, n is 2; p is 4; and R¹ is hydroxy or sulfamoylamino. In some examples, n is 3; p is 2; and R¹ is hydroxy or sulfamoylamino. In some examples, n is 3; p is 3; and R¹ is hydroxy or sulfamoylamino. In some examples, n is 3; p is 4; and R¹ is hydroxy or sulfamoylamino. In some examples, n is 4; p is 2; and R¹ is hydroxy or sulfamoylamino. In some examples, n is 4; p is 3; and R¹ is hydroxy or sulfamoylamino. In some examples, n is 4; p is 4; and R¹ is hydroxy or sulfamoylamino. In some examples, R⁷ is hydrogen. In some examples, R⁷ is hydrogen, and R¹ is hydroxy, sulfamoylamino, or sulfamic acid. In some examples, R² and R³ are hydrogen, R⁴ is 1-phenylvinyl or 1-phenylethyl, and R⁵ is phenyl and R⁷ is hydrogen. In some examples, R¹ is hydroxy, alkyl, amino, sulfamoylamino, aminoalkyl, carbamoyl, sulphate, sulfonate, aminosulfonyl, phosphate, phosphonate, or heterocyclyl, wherein R¹ is optionally substituted with R¹⁰; and R⁷ is hydrogen.

Optionally, the compounds of Formula I can be represented by Structure I-B as shown below:

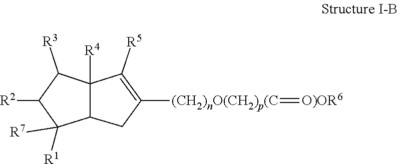

including prodrugs, salts, and stereoisomers thereof.

In Structure I-B, n, p, R¹, R², R³, R₄, R⁵, R⁷, and R¹¹, are as defined above for Formula I.

Also in Structure I-B, R⁶ is hydrogen or alkyl, wherein R⁶ is optionally terminally substituted with a hydroxy, carboxy, or phosphate, wherein the hydroxy, carboxy, or phosphate are optionally further substituted with R¹⁰.

Additionally in Structure I-B, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹ (as defined in Formula I).

In some examples of Structure I-B, n is 2, 3, or 4; p is 2, 3, or 4; and R¹ is hydroxy or sulfamoylamino. In some examples, n is 2; p is 2, 3, or 4; and R¹ is hydroxy or sulfamoylamino. In some examples, n is 3; p is 2, 3, or 4; and R¹ is hydroxy or sulfamoylamino. In some examples, n is 4; p is 2, 3, or 4; and R¹ is hydroxy or sulfamoylamino. In some examples, n is 2; p is 2; and R¹ is hydroxy or sulfamoylamino. In some examples, n is 3; p is 3; and R¹ is hydroxy or sulfamoylamino. In some examples, n is 4; p is 4; and R¹ is hydroxy or sulfamoylamino. In some examples, n is 2; p is 3; and R¹ is hydroxy or sulfamoylamino. In some examples, n is 2; p is 4; and R¹ is hydroxy or sulfamoylamino. In some examples, n is 3; p is 2; and R¹ is hydroxy or sulfamoylamino. In some examples, n is 3; p is 4; and R¹ is hydroxy or sulfamoylamino. In some examples, n is 4; p is 2; and R¹ is hydroxy or sulfamoylamino. In some examples, n is 4; p is 3; and R¹ is hydroxy or sulfamoylamino. In some examples, n is 4; p is 4; and R¹ is hydroxy or sulfamoylamino. In some examples, R⁷ is hydrogen. In some examples, R⁷ is hydrogen, and R¹ is hydroxy, sulfamoylamino, or sulfamic acid. In some examples, R² and R³ are hydrogen, R⁴ is 1-phenylvinyl or 1-phenylethyl, and R⁵ is phenyl and R⁷ is hydrogen. In some examples, R⁶ is hydrogen. In some examples, R⁶ is alkyl, methyl, or ethyl. In some examples, R⁶ is alkyl optionally substituted with a hydroxy, carboxy, or phosphate, wherein the hydroxy, carboxy, or phosphate are optionally further substituted with R¹⁰. In some examples, R¹ is hydroxy, alkyl, amino, sulfamoylamino, aminoalkyl, carbamoyl, sulphate, sulfonate, aminosulfonyl, phosphate, phosphonate, or heterocyclyl, wherein R¹ is optionally substituted with R¹⁰; and R⁷ is hydrogen.

In some examples of Structure I-B, n is 2, 3, or 4; p is 2, 3, or 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 2, 3, or 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 2, 3, or 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 2, 3, or 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen.

Optionally, the compounds of Formula I can be represented by Structure I-C as shown below:

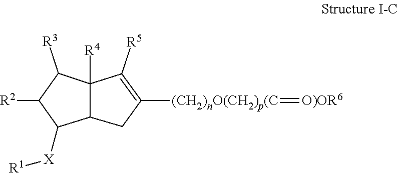

including prodrugs, salts, and stereoisomers thereof.

In Structure I-C, n, p, R², R³, R₄, R⁵, and R¹¹, are as defined above for Formula I. Also in Structure I-C, X is a linking group, —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—.

Additionally in Structure I-C, R¹ is hydrogen, hydroxy, alkyl, alkanoyl, amino, aminoalkyl, sulfamoylamino, carbamoyl, sulfate, sulfonate, aminosulfonyl, phosphate, phosphonate, or heterocyclyl.

Optionally in Structure I-C, X and R¹ may be as follows: a) X is O, and R¹ is alkanoyl; b) X is-NH—, and R¹ is alkanoyl; c) X is O, and R¹ is aminosulfonyl; d) X is-NH—, and R¹ is aminosulfonyl; or e) X is —(C═O)—, and R¹ is amino.

Further, in Structure I-C, R⁶ is a hydrogen or alkyl wherein R⁶ is optionally terminally substituted with a hydroxy, carboxy, or phosphate, wherein the hydroxy, carboxy, or phosphate are optionally further substituted with R¹⁰.

Additionally, in Structure I-C, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹ (which is defined in Formula I).

In some examples of Structure I-C, n is 2, 3, or 4; p is 2, 3, or 4. In some examples, n is 2; p is 2, 3, or 4. In some examples, n is 3; p is 2, 3, or 4. In some examples, n is 4; p is 2, 3, or 4. In some examples, n is 2; p is 2. In some examples, n is 3; p is 3. In some examples, n is 4; p is 4. In some examples, n is 2; p is 3. In some examples, n is 2; p is 4. In some examples, nis 3; p is 2. In some examples, n is 3; p is 4. In some examples, n is 4; p is 2. In some examples, n is 4; p is 3.

In some examples of Structure I-C, R⁶ is hydrogen. In some examples, R⁶ is alkyl, methyl, or ethyl. In some examples, R² and R³ are hydrogen, R⁴ is 1-phenylvinyl or 1-phenylethyl, and R⁵ is phenyl. In some examples, R² and R³ are hydrogen, R⁴ is 1-phenylvinyl or 1-phenylethyl, and R⁵ is phenyl, and R⁶ is hydrogen. In some examples, R² and R³ are hydrogen, R⁴ is 1-phenylvinyl or 1-phenylethyl, and R⁵ is phenyl, and R⁶ is alkyl, methyl, or ethyl. In some examples, R⁶ is hydrogen or alkyl optionally terminally substituted with a hydroxy, carboxy, or phosphate, wherein the hydroxy, carboxy, or phosphate are optionally further substituted with R¹⁰. In some examples, R¹ is hydroxy or sulfamoylamino, wherein R¹ is optionally substituted with R¹⁰, R² and R³ are hydrogen, R⁴ is 1-phenylvinyl or 1-phenylethyl, and R⁵ is phenyl, and R⁶ is hydrogen. In some examples, R¹ is hydroxy or sulfamoylamino, wherein R¹ is optionally substituted with R¹⁰, R² and R³ are hydrogen, R⁴ is 1-phenylvinyl or 1-phenylethyl, and R⁵ is phenyl, and R⁶ is alkyl, methyl, or ethyl. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰, R² and R³ are hydrogen, R⁴ is 1-phenylvinyl or 1-phenylethyl, and R⁵ is phenyl, and R⁶ is hydrogen. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰, R² and R³ are hydrogen, R⁴ is 1-phenylvinyl or 1-phenylethyl, and R⁵ is phenyl, and R⁶ is alkyl, methyl, or ethyl.

Optionally, the compounds of Formula I can be represented by Structure I-D as shown below:

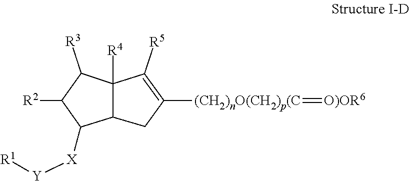

including prodrugs, salts, and stereoisomers thereof.

In Structure I-D, n, p, R², R³, R₄, R⁵, and R¹¹, are as defined above for Formula I.

Also in Structure I-D, X is a linking group, —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—.

Further in Structure I-D, Y is —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—.

Additionally in Structure I-D, R¹ is hydrogen, hydroxy, alkyl, alkanoyl, amino, aminoalkyl, sulfamoylamino, carbamoyl, sulfate, sulfonate, aminosulfonyl, phosphate, phosphonate, or heterocyclyl.

Optionally in Structure I-D, X, Y, and R¹ may be as follows: a) X is O, and R¹ is alkanoyl; b) X is-NH—, and R¹ is alkanoyl; c) X is O, and R¹ is aminosulfonyl; d) X is-NH—, and R¹ is aminosulfonyl; e) X is —(C═O)—, and R¹ is amino; or f) X is O, Y is —(C═O)—, R¹ is amino.

Further, in Structure I-D, R⁶ is a hydrogen or alkyl wherein R⁶ is optionally terminally substituted with a hydroxy, carboxy, or phosphate, wherein the hydroxy, carboxy, or phosphate are optionally further substituted with R¹⁰.

Additionally, in Structure I-D, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹ (which is defined in Formula I).

In some examples of Structure I-D, n is 2, 3, or 4; p is 2, 3, or 4. In some examples, n is 2; p is 2, 3, or 4. In some examples, n is 3; p is 2, 3, or 4. In some examples, n is 4; p is 2, 3, or 4. In some examples, n is 2; p is 2. In some examples, n is 3; p is 3. In some examples, n is 4; p is 4. In some examples, n is 2; p is 3. In some examples, n is 2; p is 4. In some examples, nis 3; p is 2. In some examples, n is 3; p is 4. In some examples, n is 4; p is 2. In some examples, n is 4; p is 3.

In some examples of Structure I-D, R⁶ is hydrogen. In some examples, R⁶ is alkyl, methyl, or ethyl. In some examples, R² and R³ are hydrogen, R⁴ is 1-phenylvinyl or 1-phenylethyl, and R⁵ is phenyl. In some examples, R² and R³ are hydrogen, R⁴ is 1-phenylvinyl or 1-phenylethyl, and R⁵ is phenyl, and R⁶ is hydrogen. In some examples, R² and R³ are hydrogen, R⁴ is 1-phenylvinyl or 1-phenylethyl, and R⁵ is phenyl, and R⁶ is alkyl, methyl, or ethyl. In some examples, R⁶ is hydrogen or alkyl optionally terminally substituted with a hydroxy, carboxy, or phosphate, wherein the hydroxy, carboxy, or phosphate are optionally further substituted with R¹⁰. In some examples, R¹ is hydroxy or sulfamoylamino, wherein R¹ is optionally substituted with R¹⁰, R² and R³ are hydrogen, R⁴ is 1-phenylvinyl or 1-phenylethyl, and R⁵ is phenyl, and R⁶ is hydrogen. In some examples, R¹ is hydroxy or sulfamoylamino, wherein R¹ is optionally substituted with R¹⁰, R² and R³ are hydrogen, R⁴ is 1-phenylvinyl or 1-phenylethyl, and R⁵ is phenyl, and R⁶ is alkyl, methyl, or ethyl. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰, R² and R³ are hydrogen, R⁴ is 1-phenylvinyl or 1-phenylethyl, and R⁵ is phenyl, and R⁶ is hydrogen. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰, R² and R³ are hydrogen, R⁴ is 1-phenylvinyl or 1-phenylethyl, and R⁵ is phenyl, and R⁶ is alkyl, methyl, or ethyl.

Optionally, the compounds of Formula I can be represented by Structure I-E as shown below:

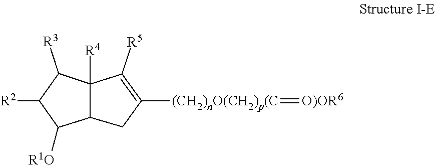

including prodrugs, salts, and stereoisomers thereof.

In Structure I-E, n, p, R², R³, R₄, R⁵, and R¹¹, are as defined above for Formula I.

Also in Structure I-E, R¹ is hydrogen, alkyl, halogen, nitro, cyano, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰.

Additionally in Structure I-E, R⁶ is a lipid or alkyl wherein R⁶ is optionally terminally substituted with a hydroxy, carboxy, or phosphate, wherein the hydroxy, carboxy, or phosphate are optionally further substituted with R¹⁰.

Further in Structure I-E, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹.

In some examples of Structure I-E, n is 2, 3, or 4; p is 2, 3, or 4. In some examples, n is 2; p is 2, 3, or 4. In some examples, n is 3; p is 2, 3, or 4. In some examples, n is 4; p is 2, 3, or 4. In some examples, n is 2; p is 2. In some examples, n is 3; p is 3. In some examples, n is 4; p is 4. In some examples, n is 2; p is 3. In some examples, n is 2; p is 4. In some examples, nis 3; p is 2. In some examples, n is 3; p is 4. In some examples, n is 4; p is 2. In some examples, n is 4; p is 3.

In some examples of Structure I-E, n is 2, 3, or 4; p is 2, 3, or 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; R² and R³ are hydrogen, R⁴ is 1-phenylvinyl or 1-phenylethyl, and R⁵ is phenyl, R⁶ is hydrogen, alkyl, methyl, or ethyl.

Optionally, the compounds of Formula I can be represented by Structure I-F as shown below:

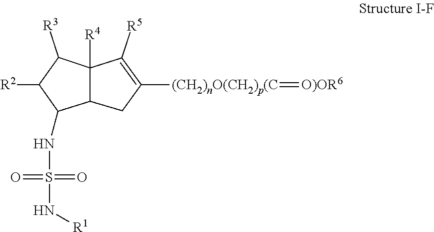

including prodrugs, salts, and stereoisomers thereof.

In Structure I-F, n, p, R², R³, R₄, R⁵, and R¹¹, are as defined above for Formula I.

Also in Structure I-F, R¹ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰.

Additionally in Structure I-F, R⁶ is a lipid or alkyl wherein R⁶ is optionally terminally substituted with a hydroxy, carboxy, or phosphate, wherein the hydroxy, carboxy, or phosphate are optionally further substituted with R¹⁰.

Further in Structure I-F, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹.

In some examples of Structure I-F, n is 2, 3, or 4; p is 2, 3, or 4. In some examples, n is 2; p is 2, 3, or 4. In some examples, n is 3; p is 2, 3, or 4. In some examples, n is 4; p is 2, 3, or 4. In some examples, n is 2; p is 2. In some examples, n is 3; p is 3. In some examples, n is 4; p is 4. In some examples, n is 2; p is 3. In some examples, n is 2; p is 4. In some examples, nis 3; p is 2. In some examples, n is 3; p is 4. In some examples, n is 4; p is 2. In some examples, n is 4; p is 3. In some examples, n is 2, 3, or 4; p is 2, 3, or 4. In some examples, n is 2; p is 2, 3, or 4. In some examples, n is 3; p is 2, 3, or 4. In some examples, n is 4; p is 2, 3, or 4. In some examples, n is 2; p is 2. In some examples, n is 3; p is 3. In some examples, n is 4; p is 4. In some examples, n is 2; p is 3. In some examples, n is 2; p is 4. In some examples, n is 3; p is 2. In some examples, n is 3; p is 4. In some examples, n is 4; p is 2. In some examples, n is 4; p is 3.

In some examples of Structure I-F, n is 2, 3, or 4; p is 2, 3, or 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; R² and R³ are hydrogen, R⁴ is 1-phenylvinyl or 1-phenylethyl, and R⁵ is phenyl, R⁶ is hydrogen, alkyl, methyl, or ethyl.

A class of compounds described herein is represented by Formula II:

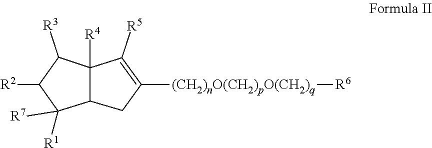

including prodrugs, salts, and stereoisomers thereof.

In Formula II, n is 1-6. For example, n can be from 2-4.

Also in Formula II, p is 1-6. For example, p can be from 2-4.

Additionally in Formula II, q is 1-6. For example, q can be from 2-4.

Also in Formula II, R¹ is alkyl, halogen, nitro, cyano, hydroxy, amino, sulfamoylamino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R¹ can be substituted with one or more, the same or different, R¹⁰ (as described below).

Further in Formula II, R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R² can be substituted with one or more, the same or different, R¹⁰.

Also in Formula II, R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R³ can be substituted with one or more, the same or different, R¹⁰;

Additionally in Formula II, R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R⁴ can be substituted with one or more, the same or different, R¹⁰.

Further in Formula II, R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R⁵ can be substituted with one or more, the same or different, R¹⁰.

Also in Formula II, R⁶ is a hydrogen, carboxy, or alkyl. In some examples, R⁶ is optionally terminally substituted with a hydroxy, carboxy, or phosphate. In some examples, R⁶ is optionally substituted with a hydroxy, carboxy, or phosphate. Optionally, the hydroxy, carboxy, or phosphate are further substituted with R¹⁰.

Additionally in Formula II, R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. Optionally, R⁷ is substituted with one or more, the same or different, R¹⁰.

In some cases of Formula II, R¹ and R⁷ together are an oxo or oxime. The oxime can be optionally substituted with one or more, the same or different, R¹⁰.

In Formula II, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl. In some cases, R¹⁰ can be aminosulfonyl. Optionally, R¹⁰ can be optionally substituted with one or more, the same or different, R¹¹.

Also in Formula II, R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

In some examples of Formula II, n is 2, 3, or 4; p is 2, 3, or 4. In some examples, n is 2; p is 2, 3, or 4. In some examples, n is 3; p is 2, 3, or 4. In some examples, n is 4; p is 2, 3, or 4. In some examples, n is 2; p is 2. In some examples, n is 3; p is 3. In some examples, n is 4; p is 4. In some examples, n is 2; p is 3. In some examples, n is 2; p is 4. In some examples, n is 3; p is 2. In some examples, n is 3; p is 4. In some examples, n is 4; p is 2. In some examples, n is 4; p is 3.

In some examples of Formula II, n is 2, 3, or 4; p is 2, 3, or 4; q is 2, 3, or 4. In some examples, n is 2; p is 2, 3, or 4; q is 2, 3, or 4. In some examples, n is 3; p is 2, 3, or 4; q is 2, 3, or 4. In some examples, n is 4; p is 2, 3, or 4; q is 2, 3, or 4. In some examples, n is 2; p is 2; q is 2, 3, or 4. In some examples, n is 3; p is 3; q is 2, 3, or 4. In some examples, n is 4; p is 4; q is 2, 3, or 4. In some examples, n is 2; p is 2; q is 2. In some examples, n is 2; p is 3; q is 2. In some examples, n is 2; p is 3; q is 3. In some examples, n is 2; p is 3; q is 4. In some examples, n is 2; p is 4; q is 4. In some examples, n is 3; p is 2; q is 2. In some examples, n is 3; p is 3; q is 3. In some examples, n is 3; p is 4; q is 4. In some examples, n is 4; p is 2; q is 2. In some examples, n is 4; p is 3; q is 3. In some examples, n is 4; p is 4; q is 4.

In some examples of Formula II, R¹ is hydroxy or sulfamoylamino. In some examples, R⁷ is hydrogen. In some examples, R⁷ is hydrogen, and R¹ is hydroxy, sulfamoylamino, or sulfamic acid. In some examples, R² and R³ are hydrogen, R⁴ is 1-phenylvinyl or 1-phenylethyl, and R⁵ is phenyl and R⁷ is hydrogen. In some examples, R⁶ is alkyl terminally substituted with a hydroxy, carboxy, or phosphate, wherein the hydroxy, carboxy, or phosphate are optionally further substituted with R¹⁰. In some examples, R¹ is hydroxy, alkyl, amino, sulfamoylamino, aminoalkyl, carbamoyl, sulphate, sulfonate, aminosulfonyl, phosphate, phosphonate, or heterocyclyl, wherein R¹ is optionally substituted with R¹⁰; and R⁷ is hydrogen.

Optionally, the compounds of Formula II can be represented by Structure II-A as shown below:

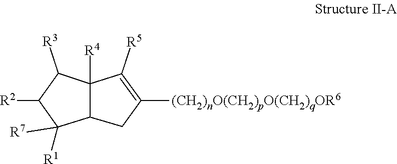

including prodrugs, salts, and stereoisomers thereof.

In Structure II-A, n, p, q, R¹, R², R³, R₄, R⁵, R⁷, and R¹¹, are as defined above for Formula II.

Also in Structure II-A, R⁶ is hydrogen, carboxy, or alkyl, wherein R⁶ is optionally substituted with an alkyl, hydroxy, carboxy, or phosphate, wherein the hydroxy, carboxy, or phosphate are optionally further substituted with R¹⁰.

Additionally in Structure II-A, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, (alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹ (as defined in Formula II).

In some examples of Structure II-A, n is 2, 3, or 4; p is 2, 3, or 4; and R¹ is hydroxy or sulfamoylamino. In some examples, n is 2; p is 2, 3, or 4; and R¹ is hydroxy or sulfamoylamino. In some examples, n is 3; p is 2, 3, or 4; and R¹ is hydroxy or sulfamoylamino. In some examples, n is 4; p is 2, 3, or 4; and R¹ is hydroxy or sulfamoylamino. In some examples, n is 2; p is 2; and R¹ is hydroxy or sulfamoylamino. In some examples, n is 3; p is 3; and R¹ is hydroxy or sulfamoylamino. In some examples, n is 4; p is 4; and R¹ is hydroxy or sulfamoylamino. In some examples, n is 2; p is 2; and R¹ is hydroxy or sulfamoylamino. In some examples, n is 2; p is 3; and R¹ is hydroxy or sulfamoylamino. In some examples, n is 2; p is 3; and R¹ is hydroxy or sulfamoylamino. In some examples, n is 2; p is 3; and R¹ is hydroxy or sulfamoylamino. In some examples, n is 2; p is 4; and R¹ is hydroxy or sulfamoylamino. In some examples, n is 3; p is 2; and R¹ is hydroxy or sulfamoylamino. In some examples, n is 3; p is 3; and R¹ is hydroxy or sulfamoylamino. In some examples, n is 3; p is 4; and R¹ is hydroxy or sulfamoylamino. In some examples, n is 4; p is 2; and R¹ is hydroxy or sulfamoylamino. In some examples, n is 4; p is 3; and R¹ is hydroxy or sulfamoylamino. In some examples, n is 4; p is 4; and R¹ is hydroxy or sulfamoylamino. In some examples, R⁷ is hydrogen. In some examples, R⁷ is hydrogen, and R¹ is hydroxy, sulfamoylamino, or sulfamic acid. In some examples, R² and R³ are hydrogen, R⁴ is 1-phenylvinyl or 1-phenylethyl, and R⁵ is phenyl and R⁷ is hydrogen. In some examples, R¹ is hydroxy, alkyl, amino, sulfamoylamino, aminoalkyl, carbamoyl, sulphate, sulfonate, aminosulfonyl, phosphate, phosphonate, or heterocyclyl, wherein R¹ is optionally substituted with R¹⁰, and R⁷ is hydrogen.

In some examples of Structure II-A, n is 2, 3, or 4; p is 2, 3, or 4; q is 2, 3, or 4; and R¹ is hydroxy or sulfamoylamino. In some examples, n is 2; p is 2, 3, or 4; q is 2, 3, or 4; and R¹ is hydroxy or sulfamoylamino. In some examples, n is 3; p is 2, 3, or 4; q is 2, 3, or 4; and R¹ is hydroxy or sulfamoylamino. In some examples, n is 4; p is 2, 3, or 4; q is 2, 3, or 4; and R¹ is hydroxy or sulfamoylamino. In some examples, n is 2; p is 2; q is 2, 3, or 4; and R¹ is hydroxy or sulfamoylamino. In some examples, n is 3; p is 3; q is 2, 3, or 4; and R¹ is hydroxy or sulfamoylamino. In some examples, n is 4; p is 4; q is 2, 3, or 4; and R¹ is hydroxy or sulfamoylamino. In some examples, n is 2; p is 2; q is 2; and R¹ is hydroxy or sulfamoylamino. In some examples, n is 2; p is 3; q is 2; and R¹ is hydroxy or sulfamoylamino. In some examples, n is 2; p is 3; q is 3; and R¹ is hydroxy or sulfamoylamino. In some examples, n is 2; p is 3; q is 4; and R¹ is hydroxy or sulfamoylamino. In some examples, n is 2; p is 4; q is 4; and R¹ is hydroxy or sulfamoylamino. In some examples, n is 3; p is 2; q is 2; and R¹ is hydroxy or sulfamoylamino. In some examples, n is 3; p is 3; q is 3; and R¹ is hydroxy or sulfamoylamino. In some examples, n is 3; p is 4; q is 4; and R¹ is hydroxy or sulfamoylamino. In some examples, n is 4; p is 2; q is 2; and R¹ is hydroxy or sulfamoylamino. In some examples, n is 4; p is 3; q is 3; and R¹ is hydroxy or sulfamoylamino. In some examples, n is 4; p is 4; q is 4; and R¹ is hydroxy or sulfamoylamino. In some examples, R⁷ is hydrogen. In some examples, R⁷ is hydrogen, and R¹ is hydroxy, sulfamoylamino, or sulfamic acid. In some examples, R² and R³ are hydrogen, R⁴ is 1-phenylvinyl or 1-phenylethyl, and R⁵ is phenyl and R⁷ is hydrogen. In some examples, R¹ is hydroxy, alkyl, amino, sulfamoylamino, aminoalkyl, carbamoyl, sulphate, sulfonate, aminosulfonyl, phosphate, phosphonate, or heterocyclyl, wherein R¹ is optionally substituted with R¹⁰; and R⁷ is hydrogen.

Optionally, the compounds of Formula II can be represented by Structure II-B as shown below:

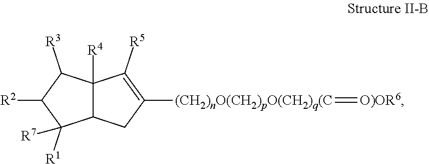

including prodrugs, salts, and stereoisomers thereof.

In Structure II-B, n, p, q, R¹, R², R³, R₄, R⁵, R⁷, and R¹¹, are as defined above for Formula II.

Also in Structure II-B, R⁶ is a hydrogen or alkyl wherein R⁶ is optionally terminally substituted with a hydroxy, carboxy, or phosphate, wherein the hydroxy, carboxy, or phosphate are optionally further substituted with R¹⁰.

Additionally in Structure II-B, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹.

In some examples of Structure II-B, n is 2, 3, or 4; p is 2, 3, or 4; q is 2, 3, or 4; and R¹ is hydroxy or sulfamoylamino. In some examples, n is 2; p is 2, 3, or 4; q is 2, 3, or 4; and R¹ is hydroxy or sulfamoylamino. In some examples, n is 3; p is 2, 3, or 4; q is 2, 3, or 4; and R¹ is hydroxy or sulfamoylamino. In some examples, n is 4; p is 2, 3, or 4; q is 2, 3, or 4; and R¹ is hydroxy or sulfamoylamino. In some examples, n is 2; p is 2; q is 2, 3, or 4; and R¹ is hydroxy or sulfamoylamino. In some examples, n is 3; p is 3; q is 2, 3, or 4; and R¹ is hydroxy or sulfamoylamino. In some examples, n is 4; p is 4; q is 2, 3, or 4; and R¹ is hydroxy or sulfamoylamino. In some examples, n is 2; p is 2; q is 2; and R¹ is hydroxy or sulfamoylamino. In some examples, n is 2; p is 3; q is 2; and R¹ is hydroxy or sulfamoylamino. In some examples, n is 2; p is 3; q is 3; and R¹ is hydroxy or sulfamoylamino. In some examples, n is 2; p is 3; q is 4; and R¹ is hydroxy or sulfamoylamino. In some examples, n is 2; p is 4; q is 4; and R¹ is hydroxy or sulfamoylamino. In some examples, n is 3; p is 2; q is 2; and R¹ is hydroxy or sulfamoylamino. In some examples, n is 3; p is 3; q is 3; and R¹ is hydroxy or sulfamoylamino. In some examples, n is 3; p is 4; q is 4; and R¹ is hydroxy or sulfamoylamino. In some examples, n is 4; p is 2; q is 2; and R¹ is hydroxy or sulfamoylamino. In some examples, n is 4; p is 3; q is 3; and R¹ is hydroxy or sulfamoylamino. In some examples, n is 4; p is 4; q is 4; and R¹ is hydroxy or sulfamoylamino. In some examples, R⁷ is hydrogen, and R¹ is hydroxy, sulfamoylamino, or sulfamic acid.

In some examples of Structure II-B, R⁶ is hydrogen. In some examples, R⁶ is alkyl, methyl, or ethyl. In some examples, R⁷ is hydrogen. In some examples, R² and R³ are hydrogen, R⁴ is 1-phenylvinyl or 1-phenylethyl, and R⁵ is phenyl and R⁷ is hydrogen. In some examples, R⁶ is hydrogen or alkyl optionally terminally substituted with a hydroxy, carboxy, or phosphate, wherein the hydroxy, carboxy, or phosphate are optionally further substituted with R¹⁰. In some examples, R¹ is hydroxy, alkyl, amino, sulfamoylamino, aminoalkyl, carbamoyl, sulphate, sulfonate, aminosulfonyl, phosphate, phosphonate, or heterocyclyl, wherein R¹ is optionally substituted with R¹⁰; and R⁷ is hydrogen. In some examples, n is 2, 3, or 4; p is 2, 3, or 4; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 2, 3, or 4; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 2, 3, or 4; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 2, 3, or 4; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 2; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen.

In some examples of Structure II-B, n is 3; p is 3; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 4; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen.

In some examples, n is 2; p is 3; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 4; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 2; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 4; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen.

In some examples of Structure II-B, n is 4; p is 2; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 3; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 4; q is 2; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2, 3, or 4; p is 2, 3, or 4; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 2, 3, or 4; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 2, 3, or 4; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 2, 3, or 4; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 2; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen.

In some examples of Structure II-B, n is 3; p is 3; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 4; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 3; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 4; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 2; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 4; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 2; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 3; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 4; q is 3; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen.

In some examples of Structure II-B, n is 2, 3, or 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 2, 3, or 4; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 2, 3, or 4; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 2, 3, or 4; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 2; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 3; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 4; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen.

In some examples of Structure II-B, n is 2; p is 3; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 2; p is 4; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 2; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 3; p is 4; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 2; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 3; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen. In some examples, n is 4; p is 4; q is 4; R¹ is hydroxy or sulfamoylamino; R² and R³ are hydrogen, R⁴ is 1-phenylvinyl or 1-phenylethyl, and R⁵ is phenyl, R⁶ is hydrogen, alkyl, methyl, or ethyl; and R⁷ is hydrogen.

Optionally, the compounds of Formula II can be represented by Structure II-C as shown below:

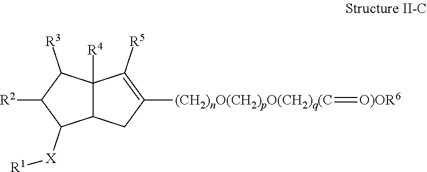

including prodrugs, salts, and stereoisomers thereof.

In Structure II-C, n, p, q, R², R³, R₄, R⁵, and R¹¹, are as defined above for Formula II.

Also in Structure II-C, X is a linking group, —CH₂—, —C(OH) (OH)—, —C(OH) H, —C(Hal) (Hal)-, —C(Hal) H—, —O—, —S—, —(S═O)—, —SO₂—, —NH—, —(C—O)—, —(C═NH)—, or —(C═S)—.

Additionally in Structure II-C, R¹ is hydrogen, hydroxy, alkyl, alkanoyl, amino, aminoalkyl, sulfamoylamino, carbamoyl, sulfate, sulfonate, aminosulfonyl, phosphate, phosphonate, or heterocyclyl.

Optionally in Structure II-C, X and R¹ may be as follows: a) X is O, and R¹ is alkanoyl; b) X is-NH—, and R¹ is alkanoyl; c) X is O, and R¹ is aminosulfonyl; d) X is-NH—, and R¹ is aminosulfonyl; or e) X is —(C—O)—, and R¹ is amino.

Further, in Structure II-C, R⁶ is a hydrogen or alkyl wherein R⁶ is optionally terminally substituted with a hydroxy, carboxy, or phosphate, wherein the hydroxy, carboxy, or phosphate are optionally further substituted with R¹⁰.

Additionally, in Structure II-C, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹ (which is defined in Formula II).

In some examples of Structure II-C, n is 2, 3, or 4; p is 2, 3, or 4. In some examples, n is 2; p is 2, 3, or 4. In some examples, n is 3; p is 2, 3, or 4. In some examples, n is 4; p is 2, 3, or 4. In some examples, n is 2; p is 2. In some examples, n is 3; p is 3. In some examples, n is 4; p is 4. In some examples, n is 2; p is 3. In some examples, n is 2; p is 4. In some examples, n is 3; p is 2. In some examples, n is 3; p is 4. In some examples, n is 4; p is 2. In some examples, n is 4; p is 3.

In some examples of Structure II-C, n is 2, 3, or 4; p is 2, 3, or 4; q is 2, 3, or 4. In some examples, n is 2; p is 2, 3, or 4; q is 2, 3, or 4. In some examples, n is 3; p is 2, 3, or 4; q is 2, 3, or 4. In some examples, n is 4; p is 2, 3, or 4; q is 2, 3, or 4. In some examples, n is 2; p is 2; q is 2, 3, or 4. In some examples, n is 3; p is 3; q is 2, 3, or 4. In some examples, n is 4; p is 4; q is 2, 3, or 4. In some examples, n is 2; p is 2; q is 2. In some examples, n is 2; p is 3; q is 2. In some examples, n is 2; p is 3; q is 3. In some examples, n is 2; p is 3; q is 4. In some examples, n is 2; p is 4; q is 4. In some examples, n is 3; p is 2; q is 2. In some examples, n is 3; p is 3; q is 3. In some examples, n is 3; p is 4; q is 4. In some examples, n is 4; p is 2; q is 2. In some examples, n is 4; p is 3; q is 3. In some examples, n is 4; p is 4; q is 4. In some examples, R⁶ is hydrogen. In some examples, R⁶ is alkyl, methyl, or ethyl.

In some examples of Structure II-C, R² and R³ are hydrogen, R⁴ is 1-phenylvinyl or 1-phenylethyl, and R⁵ is phenyl. In some examples, R² and R³ are hydrogen, R⁴ is 1-phenylvinyl or 1-phenylethyl, and R⁵ is phenyl, and R⁶ is hydrogen. In some examples, R² and R³ are hydrogen, R⁴ is 1-phenylvinyl or 1-phenylethyl, and R⁵ is phenyl, and R⁶ is alkyl, methyl, or ethyl. In some examples, R⁶ is hydrogen or alkyl optionally terminally substituted with a hydroxy, carboxy, or phosphate, wherein the hydroxy, carboxy, or phosphate are optionally further substituted with R¹⁰. In some examples, R¹ is hydroxy or sulfamoylamino, wherein R¹ is optionally substituted with R¹⁰, R² and R³ are hydrogen, R⁴ is 1-phenylvinyl or 1-phenylethyl, and R⁵ is phenyl, and R⁶ is hydrogen. In some examples, R¹ is hydroxy or sulfamoylamino, wherein R¹ is optionally substituted with R¹⁰, R² and R³ are hydrogen, R⁴ is 1-phenylvinyl or 1-phenylethyl, and R⁵ is phenyl, and R⁶ is alkyl, methyl, or ethyl. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰, R² and R³ are hydrogen, R⁴ is 1-phenylvinyl or 1-phenylethyl, and R⁵ is phenyl, and R⁶ is hydrogen. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰, R² and R³ are hydrogen, R⁴ is 1-phenylvinyl or 1-phenylethyl, and R⁵ is phenyl, and R⁶ is alkyl, methyl, or ethyl.

In some examples of Structure II-C, n is 2, 3, or 4; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 2; R² and R³ are hydrogen, R⁴ is 1-phenylvinyl or 1-phenylethyl, and R⁵ is phenyl, Re is hydrogen, alkyl, methyl, or ethyl. In some examples, n is 2; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-C, n is 2, 3, or 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-C, n is 2; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2, 3, or 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-C, n is 2; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl In some examples, n is 4; p is 4; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2, 3, or 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-C, n is 2; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-C, n is 4; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2, 3, or 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl.

Optionally, the compounds of Formula II can be represented by Structure II-D as shown below:

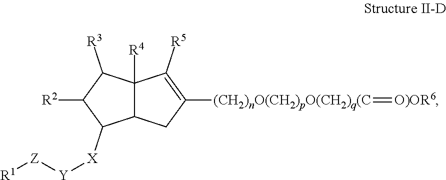

including prodrugs, salts, and stereoisomers thereof.

In Structure II-D, n, p, q, R², R³, R₄, R⁵, and R¹¹, are as defined above for Formula II.

Also in Structure II-D, X is a linking group, —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C—S)—.

Additionally in Structure II-D, Y is —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—.

Further in Structure II-D, Z is —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—.

Also in Structure II-D, R¹ is hydrogen, hydroxy, alkyl, alkanoyl, amino, aminoalkyl, sulfamoylamino, carbamoyl, sulfate, sulfonate, aminosulfonyl, phosphate, phosphonate, or heterocyclyl.

Optionally in Structure II-D, X, Y, Z, and R¹ may be as follows: a) X is O, and R¹ is alkanoyl; b) X is-NH—, and R¹ is alkanoyl; c) X is O, and R¹ is aminosulfonyl; d) X is-NH—, and R¹ is aminosulfonyl; e) X is —(C═O)—, R¹ is amino; f) X is O, Y is —(C═O)—, R¹ is amino; g) X is O, Y is —(C═O)—, Z is-NH—, and R¹ is sulfonate; and h) X is O, Y is —(C═O)—, Z is-NH—, and R¹ is aminosulfonyl.

Further, in Structure II-D, R⁶ is a hydrogen or alkyl wherein R⁶ is optionally terminally substituted with a hydroxy, carboxy, or phosphate, wherein the hydroxy, carboxy, or phosphate are optionally further substituted with R¹⁰.

Additionally, in Structure II-D, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹ (which is defined in Formula II).

In some examples of Structure II-D, n is 2, 3, or 4; p is 2, 3, or 4. In some examples, n is 2; p is 2, 3, or 4. In some examples, n is 3; p is 2, 3, or 4. In some examples, n is 4; p is 2, 3, or 4. In some examples, n is 2; p is 2. In some examples, n is 3; p is 3. In some examples, n is 4; p is 4. In some examples, n is 2; p is 3. In some examples, n is 2; p is 4. In some examples, nis 3; p is 2. In some examples, n is 3; p is 4. In some examples, n is 4; p is 2. In some examples, n is 4; p is 3.

In some examples of Structure II-D, n is 2, 3, or 4; p is 2, 3, or 4; q is 2, 3, or 4. In some examples, n is 2; p is 2, 3, or 4; q is 2, 3, or 4. In some examples, n is 3; p is 2, 3, or 4; q is 2, 3, or 4. In some examples, n is 4; p is 2, 3, or 4; q is 2, 3, or 4. In some examples, n is 2; p is 2; q is 2, 3, or 4. In some examples, n is 3; p is 3; q is 2, 3, or 4. In some examples, n is 4; p is 4; q is 2, 3, or 4. In some examples, n is 2; p is 2; q is 2. In some examples, n is 2; p is 3; q is 2. In some examples, n is 2; p is 3; q is 3. In some examples, n is 2; p is 3; q is 4. In some examples, n is 2; p is 4; q is 4. In some examples, n is 3; p is 2; q is 2. In some examples, n is 3; p is 3; q is 3. In some examples, n is 3; p is 4; q is 4. In some examples, n is 4; p is 2; q is 2. In some examples, n is 4; p is 3; q is 3. In some examples, n is 4; p is 4; q is 4. In some examples, R⁶ is hydrogen. In some examples, R⁶ is alkyl, methyl, or ethyl.

In some examples of Structure II-D, R² and R³ are hydrogen, R⁴ is 1-phenylvinyl or 1-phenylethyl, and R⁵ is phenyl. In some examples, R² and R³ are hydrogen, R⁴ is 1-phenylvinyl or 1-phenylethyl, and R⁵ is phenyl, and R⁶ is hydrogen. In some examples, R² and R³ are hydrogen, R⁴ is 1-phenylvinyl or 1-phenylethyl, and R⁵ is phenyl, and R⁶ is alkyl, methyl, or ethyl. In some examples, R⁶ is hydrogen or alkyl optionally terminally substituted with a hydroxy, carboxy, or phosphate, wherein the hydroxy, carboxy, or phosphate are optionally further substituted with R¹⁰. In some examples, R¹ is hydroxy or sulfamoylamino, wherein R¹ is optionally substituted with R¹⁰, R² and R³ are hydrogen, R⁴ is 1-phenylvinyl or 1-phenylethyl, and R⁵ is phenyl, and R⁶ is hydrogen. In some examples, R¹ is hydroxy or sulfamoylamino, wherein R¹ is optionally substituted with R¹⁰, R² and R³ are hydrogen, R⁴ is 1-phenylvinyl or 1-phenylethyl, and R⁵ is phenyl, and R⁶ is alkyl, methyl, or ethyl. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰, R² and R³ are hydrogen, R⁴ is 1-phenylvinyl or 1-phenylethyl, and R⁵ is phenyl, and R⁶ is hydrogen. In some examples, R¹ is hydrogen, hydroxy, alkyl, amino, aminoalkyl, carbamoyl, sulphate, sulfonate, aminosulfonyl, phosphate, phosphonate, or heterocyclyl, wherein R¹ is optionally substituted with R¹⁰, R² and R³ are hydrogen, R⁴ is 1-phenylvinyl or 1-phenylethyl, and R⁵ is phenyl, and R⁶ is alkyl, methyl, or ethyl. In some examples of Structure II-D, n is 2, 3, or 4; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-D, n is 2, 3, or 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-D, n is 2; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2, 3, or 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-D, n is 2; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl In some examples, n is 4; p is 4; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2, 3, or 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-D, n is 2; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-D, n is 4; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2, 3, or 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl.

Optionally, the compounds of Formula II can be represented by Structure II-E as shown below:

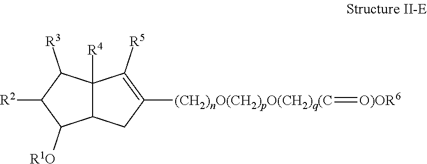

including prodrugs, salts, and stereoisomers thereof.

In Structure II-E, n, p, q, R², R³, R₄, R⁵, and R¹¹, are as defined above for Formula II.

Also in Structure II-E, R¹ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰.

Additionally in Structure II-E, R⁶ is a lipid or alkyl wherein R⁶ is optionally terminally substituted with a hydroxy, carboxy, or phosphate, wherein the hydroxy, carboxy, or phosphate are optionally further substituted with R¹⁰.

Further, in Structure II-E, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹.

In some examples of Structure II-E, n is 2, 3, or 4; p is 2, 3, or 4; q is 2, 3, or 4. In some examples, n is 2; p is 2, 3, or 4; q is 2, 3, or 4. In some examples, n is 3; p is 2, 3, or 4; q is 2, 3, or 4. In some examples, n is 4; p is 2, 3, or 4; q is 2, 3, or 4. In some examples, n is 2; p is 2; q is 2, 3, or 4. In some examples, n is 3; p is 3; q is 2, 3, or 4. In some examples, n is 4; p is 4; q is 2, 3, or 4. In some examples, n is 2; p is 2; q is 2. In some examples, n is 2; p is 3; q is 2. In some examples, n is 2; p is 3; q is 3. In some examples, n is 2; p is 3; q is 4. In some examples, n is 2; p is 4; q is 4. In some examples, n is 3; p is 2; q is 2. In some examples, n is 3; p is 3; q is 3. In some examples, n is 3; p is 4; q is 4. In some examples, n is 4; p is 2; q is 2. In some examples, n is 4; p is 3; q is 3. In some examples, n is 4; p is 4; q is 4.

In some examples of Structure II-E, n is 2, 3, or 4; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-E, n is 2; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-E, n is 2, 3, or 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-E, n is 2, 3, or 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-E, n is 2; p is 2; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl In some examples, n is 4; p is 4; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2, 3, or 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-E, n is 2; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-E, n is 2, 3, or 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl.

Optionally, the compounds of Formula II can be represented by Structure II-F as shown below:

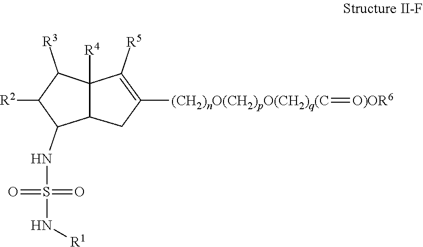

including prodrugs, salts, and stereoisomers thereof.

In Structure II-F, n, p, q, R², R³, R₄, R⁵, and R¹¹, are as defined above for Formula II. Also in Structure II-F, R¹ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰.

Additionally in Structure II-F, R⁶ is a lipid or alkyl wherein R⁶ is optionally terminally substituted with a hydroxy, carboxy, or phosphate, wherein the hydroxy, carboxy, or phosphate are optionally further substituted with R¹⁰.

Further in Structure II-F, R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹.

In some examples of Structure II-F, n is 2, 3, or 4; p is 2, 3, or 4; q is 2, 3, or 4. In some examples, n is 2; p is 2, 3, or 4; q is 2, 3, or 4. In some examples, n is 3; p is 2, 3, or 4; q is 2, 3, or 4. In some examples, n is 4; p is 2, 3, or 4; q is 2, 3, or 4. In some examples, n is 2; p is 2; q is 2, 3, or 4. In some examples, n is 3; p is 3; q is 2, 3, or 4. In some examples, n is 4; p is 4; q is 2, 3, or 4. In some examples, n is 2; p is 2; q is 2. In some examples, n is 2; p is 3; q is 2. In some examples, n is 2; p is 3; q is 3. In some examples, n is 2; p is 3; q is 4. In some examples, n is 2; p is 4; q is 4. In some examples, n is 3; p is 2; q is 2. In some examples, n is 3; p is 3; q is 3. In some examples, n is 3; p is 4; q is 4. In some examples, n is 4; p is 2; q is 2. In some examples, n is 4; p is 3; q is 3. In some examples, n is 4; p is 4; q is 4.

In some examples of Structure II-F, n is 2, 3, or 4; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 2; R² and R³ are hydrogen, R⁴ is 1-phenylvinyl or 1-phenylethyl, and R$ is phenyl, R⁶ is hydrogen, alkyl, methyl, or ethyl. In some examples, n is 3; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-F, n is 2, 3, or 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-F, n is 2, 3, or 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-F, n is 2; p is 2; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl In some examples, n is 4; p is 4; q is 2; R¹ is hydroxy; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 2; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 2; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-F, n is 2, 3, or 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 3; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 3; R² and R³ are hydrogen, R⁴ is 1-phenylvinyl or 1-phenylethyl, and R⁵ is phenyl, R⁶ is hydrogen, alkyl, methyl, or ethyl.

In some examples of Structure II-F, n is 2, 3, or 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2, 3, or 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 2; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 3; p is 4; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 2; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 3; q is 4; R² and R³ are hydrogen, R⁴ is 1-phenylvinyl or 1-phenylethyl, and R⁵ is phenyl, R⁶ is hydrogen, alkyl, methyl, or ethyl. In some examples, n is 4; p is 4; q is 4; R² and R³ are hydrogen, R⁴ is 1-phenylvinyl or 1-phenylethyl, and R⁵ is phenyl, R⁶ is hydrogen, alkyl, methyl, or ethyl.

Compounds according to the present disclosure include enantiomers, a mixture of enantiomers, a diastereomer, a mixture of two or more diastereomers, a tautomer, a mixture of two or more tautomers, or an isotopic variant thereof; or a pharmaceutically acceptable salt, solvate, or hydrate thereof.

In some examples, a compound according to the present disclosure can be selected from the compounds below, including an enantiomer, a mixture of enantiomers, a mixture of two or more diastereomers, a tautomer, a mixture of two or more tautomers, or an isotopic variant thereof, or a pharmaceutically acceptable salt, solvate, or hydrate thereof:

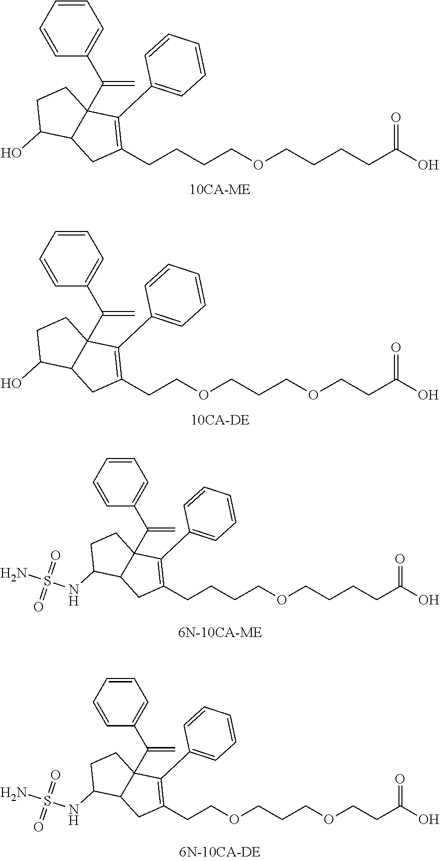 [SMILES: C=C(c1ccccc1)C12CCC(O)C1CC(CCCCOCCCCC(=O)O)=C2c1ccccc1 ; C=C(c1ccccc1)C12CCC(O)C1CC(CCOCCCOCCC(=O)O)=C2c1ccccc1 ; C=C(c1ccccc1)C12CCC(NS(N)(=O)=O)C1CC(CCCCOCCCCC(=O)O)=C2c1ccccc1 ; C=C(c1ccccc1)C12CCC(NS(N)(=O)=O)C1CC(CCOCCCOCCC(=O)O)=C2c1ccccc1]

### III. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Formula I and Formula II and other compounds as described herein include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

### IV. Pharmaceutical Compositions

While it is possible that, for use in therapy, a therapeutically effective amount of a compound disclosed herein may be administered as the raw chemical, it is typically presented as the active ingredient of a pharmaceutical composition or formulation. Accordingly, the disclosure further provides a pharmaceutical composition comprising a compound disclosed herein. The pharmaceutical composition may further comprise one or more pharmaceutically acceptable carriers, diluents, and/or excipients. The carrier(s), diluent(s) and/or excipient(s) must be acceptable in the sense of being compatible with the other ingredients of the formulation and not deleterious to the recipient thereof. In accordance with another aspect of the disclosure there is also provided a process for the preparation of a pharmaceutical formulation including admixing a compound disclosed herein with one or more pharmaceutically acceptable carriers, diluents and/or excipients.

Pharmaceutical formulations may be presented in unit dose forms containing a predetermined amount of active ingredient per unit dose. Such a unit may contain, for example, 0.5 mg to 1 g, preferably 1 mg to 700 mg, more preferably 5 mg to 100 mg of a compound disclosed herein (as a free-base, solvate (including hydrate) or salt, in any form), depending on the condition being treated, the route of administration, and the age, weight and condition of the patient. Preferred unit dosage formulations are those containing a daily dose, weekly dose, or monthly dose, of an active ingredient. Furthermore, such pharmaceutical formulations may be prepared by any of the methods well known in the pharmacy art.

Pharmaceutical formulations may be adapted for administration by any appropriate route, for example by the oral (including capsules, tablets, liquid-filled capsules, disintegrating tablets, immediate, delayed and controlled release tablets, oral strips, solutions, syrups, buccal and sublingual), rectal, nasal, inhalation, topical (including transdermal), vaginal or parenteral (including subcutaneous, intramuscular, intravenous or intradermal) route. Such formulations may be prepared by any method known in the art of pharmacy, for example by bringing into association the active ingredient with the carrier(s), excipient(s) or diluent. Generally, the carrier, excipient or diluent employed in the pharmaceutical formulation is “non-toxic,” meaning that it/they is/are deemed safe for consumption in the amount delivered in the pharmaceutical composition, and “inert” meaning that it/they does/do not appreciably react with or result in an undesired effect on the therapeutic activity of the active ingredient.

Pharmaceutical formulations adapted for oral administration may be presented as discrete units such as liquid-filled or solid capsules; immediate, delayed, or controlled release tablets; powders or granules; solutions or suspensions in aqueous or non-aqueous liquids; edible foams or whips; oil-in-water liquid emulsions, water-in-oil liquid emulsions or oral strips, such as impregnated gel strips.

For instance, for oral administration in the form of a tablet or capsule, the active drug component can be combined with an oral pharmaceutically acceptable carrier such as ethanol, glycerol, sterilized water, and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Solid capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating, slugging, and/or adding a lubricant and disintegrant and pressing into tablets. A powder mixture may be prepared by mixing the compound, suitably comminuted, with a diluent or base as described above, and optionally, with a binder such as carboxymethylcellulose, an alginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds disclosed herein can also be combined with a free-flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solutions, syrups and elixirs can be prepared in dosage unit form so that a given quantity contains a predetermined amount of the compound. Solutions and syrups can be prepared by dissolving the compound in a suitably flavored aqueous solution, while elixirs are prepared through the use of a pharmaceutically acceptable alcoholic vehicle. Suspensions can be formulated by dispersing the compound in a pharmaceutically acceptable vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, unit dosage formulations for oral administration can be microencapsulated. The formulation can also be prepared to prolong or sustain the release as for example by coating or embedding particulate material in polymers, wax or the like.

The compounds of the disclosure can also be administered in the form of liposome delivery systems, such as small unilamellar vesicles, large unilamellar vesicles and multilamellar vesicles. Liposomes can be formed from a variety of phospholipids, such as cholesterol, stearyl amine, or phosphatidylcholines.

Pharmaceutical formulations adapted for topical administration in the mouth include lozenges, pastilles, and mouth washes.

Pharmaceutical formulations adapted for rectal administration may be presented as suppositories or as enemas.

Pharmaceutical formulations adapted for nasal administration wherein the carrier is a solid include a coarse powder having a particle size for example in the range 20 to 500 microns which is administered in the manner in which snuff is taken, i.e. by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, metered dose inhalers, dry powder inhalers, nebulizers, or insufflators.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain antioxidants, buffers, bacteriostats and solutes which render the formulation of pharmaceutically acceptable tonicity with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules, and tablets.

It should be understood that in addition to the ingredients particularly mentioned above, the formulations may include other agents conventional in the art having regard to the type of formulation in question, for example, those suitable for oral administration may include flavoring agents.

### V. Methods of Use

In certain examples, this disclosure relates to methods of treating or preventing diseases or conditions associated with LRH-1 such as diabetes, cancer, or cardiovascular disease by administering an effective amount of a hexahydropentalene derivative disclosed herein to a subject in need thereof.

In certain examples, the disclosure relates to methods of treating or preventing diabetes comprising administering an effective amount of a pharmaceutical composition comprising compounds disclosed herein to a subject in need thereof. In certain examples, the subject is at risk of, exhibiting symptoms of, or diagnosed with diabetes, insulin-dependent diabetes mellitus, non insulin-dependent diabetes mellitus, or gestational diabetes.

In certain examples, this disclosure relates to compounds disclosed herein that are LRH-1 agonists for use in the prevention of progressive loss of pancreatic beta-cells. It also relates to an LRH-1 agonist for use in the preservation or restoration of pancreatic beta-cells. Further, it relates to an LRH-1 agonist for use in the prevention or treatment of type I diabetes or insulin-dependent diabetes mellitus, the increment of survival of pancreatic beta-cells, the increment of the performance of pancreatic beta-cells, the increment of the survival of a beta-cell graft, the in vitro preservation of pancreatic beta-cells, maintaining insulin secretion and/or in a method of transplanting pancreatic islet cells.

In some cases, the disclosure relates to the treatment or prevention of pancreatitis. In certain examples, the methods of treating or preventing pancreatitis comprises administering an effective amount of a pharmaceutical composition comprising a compound disclosed herein to a subject in need thereof.

Diabetes mellitus (DM) is often simply referred to as diabetes. Diabetes is a condition in which a person has a high blood sugar (glucose) level as a result of the body either not producing enough insulin, or because body cells do not properly respond to the insulin that is produced.

In healthy persons, blood glucose levels are maintained within a narrow range, primarily by the actions of the hormone insulin. Insulin is released by pancreatic beta-cells at an appropriate rate in response to circulating glucose concentrations, the response being modulated by other factors including other circulating nutrients, islet innervation and incretin hormones. Insulin maintains glucose concentrations by constraining the rate of hepatic glucose release to match the rate of glucose clearance.

Insulin thus enables body cells to absorb glucose, to turn into energy. If the body cells do not absorb the glucose, the glucose accumulates in the blood (hyperglycemia), leading to various potential medical complications. Accordingly, diabetes is characterized by increased blood glucose resulting in secondary complications such as cardiovascular diseases, kidney failure, retinopathy and neuropathy if not properly controlled. Two major pathophysiologies are related to increase glycemia. The first is an autoimmune attack against the pancreatic insulin-producing beta-cells (Type 1 diabetes or insulin-dependent diabetes) whilst the second is associated to poor beta-cell function and increased peripheral insulin resistance (Type 2 diabetes or non-insulin dependent diabetes). Similar to Type 1, beta-cell death is also observed in Type 2 diabetes. Type 1 and often Type 2 diabetes requires the person to inject insulin.

Type 1 DM is typically characterized by loss of the insulin-producing beta-cells of the islets of Langerhans in the pancreas leading to insulin deficiency. This type of diabetes can be further classified as immune-mediated or idiopathic. The majority of Type 1 diabetes is of the immune-mediated nature, where beta-cell loss is a T-cell mediated autoimmune attack. Sensitivity and responsiveness to insulin are usually normal, especially in the early stages. Type 1 diabetes can affect children or adults but was traditionally termed “juvenile diabetes” because it represents a majority of the diabetes cases in children.

Type 2 DM is characterized by beta-cell dysfunction in combination with insulin resistance. The defective responsiveness of body tissues to insulin is believed to involve the insulin receptor. Similar to Type 1 diabetes, an insufficient beta cell mass is also a pathogenic factor in many Type 2 diabetic patients. In the early stage of Type 2 diabetes, hyperglycemia can be reversed by a variety of measures and medications that improve insulin secretion and reduce glucose production by the liver. As the disease progresses, the impairment of insulin secretion occurs, and therapeutic replacement of insulin may sometimes become necessary in certain patients. In certain examples, the treatment of diabetes by administering compounds disclosed herein is in combination with the administration of insulin.

Diabetes without proper treatments can cause many complications. Acute complications include hyperglycemia, diabetic ketoacidosis, or nonketotic hyperosmolar coma. Serious long-term complications include cardiovascular disease, chronic renal failure, retinal damage. In some examples, this disclosure relates to methods of treating or preventing central nervous system (CNS) disorders, such as diabetic neuropathy. In some cases, this disclosure relates to improved mitochondrial dysfunction and a decreased level of apoptosis in podocytes. In certain examples, this disclosure relates to methods of treating or preventing cardiovascular disease comprising administering an effective amount of a pharmaceutical composition comprising a compound disclosed herein to a subject in need thereof.

In certain examples, the cardiovascular disease is coronary artery diseases (CAD), angina, myocardial infarction, stroke, hypertensive heart disease, rheumatic heart disease, cardiomyopathy, heart arrhythmia, congenital heart disease, valvular heart disease, carditis, aortic aneurysms, peripheral artery disease, and venous thrombosis.

In some examples, this disclosure relates to methods of treating or preventing diabetic nephropathy (i.e., diabetic kidney disease). In some cases, this disclosure relates to improved mitochondrial dysfunction, a decreased level of apoptosis in podocytes, and improved podocyte function/survival.

In certain examples, this disclosure relates to methods of managing cancer. “Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain examples, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

In certain examples, this disclosure relates to methods of treating cancer comprising administering an effective amount of an agent to a subject in need thereof. Optionally, the cancer is bladder cancer, brain cancer, breast cancer (e.g., triple negative breast cancer), bronchus cancer, colorectal cancer (e.g., colon cancer, rectal cancer), cervical cancer, chondrosarcoma, endometrial cancer, gastrointestinal cancer, gastric cancer, genitourinary cancer, glioblastoma, head and neck cancer, hepatic cancer, hepatocellular carcinoma, leukemia, liver cancer, lung cancer, lymphoma, melanoma of the skin, ovarian cancer, pancreatic cancer, prostate cancer, renal cancer, skin cancer, testicular cancer, thyroid cancer, or uterine cancer. Optionally, the cancer is a cancer that affects one or more of the following sites: oral cavity and pharynx (e.g., tongue, mouth, pharynx, or other oral cavity); digestive system (e.g., esophagus, stomach, small intestine, colon, rectum, anus, anal canal, anorectum, liver and intrahepatic bile duct, gallbladder and other biliary, pancreas, or other digestive organs); respiratory system (e.g., larynx, lung and bronchus, or other respiratory organs); bones and joints; soft tissue (e.g., heart); skin (e.g., melanoma of the skin or other nonepithelial skin); breast; genital system (e.g., uterine cervix, uterine corpus, ovary, vulva, vagina and other female genital areas, prostate, testis, penis and other male genital areas); urinary system (e.g., urinary bladder, kidney and renal pelvis, and ureter and other urinary organs); eye and orbit; brain and other nervous system; endocrine system (e.g., thyroid and other endocrine); lymphoma (e.g., Hodgkin lymphoma and non-Hodgkin lymphoma); myeloma; or leukemia (e.g., acute lymphocytic leukemia, chronic lymphocytic leukemia, acute myeloid leukemia, chronic myeloid leukemia, or other leukemia). Optionally, the cancer is a cancer that has an increased expression of LRH-1 as compared to non-cancerous cells of the same cell type.

In certain examples, the cancer is pancreatic cancer, breast cancer, liver cancer, colon cancer, or gastrointestinal tumors.

Benod et al. report LRH-1 regulates pancreatic cancer cell growth and proliferation. Proc Natl Acad Sci USA, 2011, 108 (41): 16927-31. Pan et al. report LRH-1-dependent programming of mitochondrial glutamine processing drives liver cancer. Genes Dev, 2016, (11): 1255-1260. Holly et al. LRH-1 drives colon cancer cell growth by repressing the expression of the CDKN1A gene in a p53-dependent manner. Nucleic Acids Res, 2016, 44 (2): 582-594.

In certain examples, the cancer is selected from bladder cancer, breast cancer, colon cancer, rectal cancer, endometrial cancer, kidney cancer, leukemia, lung cancer, melanoma, non-Hodgkin lymphoma, pancreatic cancer, prostate cancer, and thyroid cancer.

The compounds disclosed herein can be used alone in the treatment of each of the foregoing conditions or can be used to provide additive or potentially synergistic effects with certain existing chemotherapies, radiation, biological or immunotherapeutics (including monoclonal antibodies) and vaccines. The compounds disclosed herein may be useful for restoring effectiveness of certain existing chemotherapies and radiation and or increasing sensitivity to certain existing chemotherapies and/or radiation.

Coste et al. report LRH-1-mediated glucocorticoid synthesis in enterocytes protects against inflammatory bowel disease. PNAS, 2007. 104 (32)13098-13103. See also Fernandez-Marcos et al. Emerging actions of the nuclear receptor LRH-1 in the gut, Biochim Biophys Acta. 2011 August; 1812 (8): 947-955. Mueller et al. The nuclear receptor LRH-1 critically regulates extra-adrenal glucocorticoid synthesis in the intestine, Journal of Experimental Medicine September 2006, 203 (9)2057-2062.

Thus, in certain examples, this disclosure relates to methods to prevent or treat gut, intestinal, and colonic inflammation, comprising administrating a compound disclosed herein in an effective amount to a subject in need thereof. In certain examples, the subject is at risk of, exhibiting symptoms of, or diagnosed with intestinal and colonic inflammation.

In another aspect, the disclosure relates to methods to prevent or treat inflammatory bowel diseases (IBD), comprising administrating a compound disclosed herein in an effective amount to a subject in need thereof. In certain examples, the subject is at risk of, exhibiting symptoms of, or diagnosed with inflammatory bowel diseases (IBD). As outlined in Mays et al., Cell Chemical Biology, 29:1174-1186 (2022), agonists of LRH-1 improve outcomes in T-cell transfer mouse model of colitis, resulting in heightened expression of genes involved in steroidogenesis and lower expression of inflammatory cytokine genes in the gut.

Optionally, the disclosure relates to methods to prevent or treat diseases characterized by inflammation of the bowel, including celiac disease, immune checkpoint inhibitor colitis, IgA nephropathy, Crohn's disease, ulcerative colitis, regional enteritis, granulomatous enteritis, distal ileitis, regional ileitis, terminal ileitis, and others. In some cases, the disclosure relates to methods to protect tissues from inflammatory bowel disease, including ulcerative colitis and Crohn's disease.

In another aspect, the disclosure relates to methods to prevent or treat Crohn's disease, comprising administrating a compound disclosed herein in an effective amount to a subject in need thereof. In certain examples, the subject is at risk of, exhibiting symptoms of, or diagnosed with Crohn's disease.

In another aspect, the disclosure relates to methods to prevent or treat colitis or ulcerative colitis, comprising administrating a compound disclosed herein in an effective amount to a subject in need thereof. In certain examples, the subject is at risk of, exhibiting symptoms of, or diagnosed with colitis or ulcerative colitis.

Overweight and obesity are increasingly common conditions in the world. Doctors measure body mass index (BMI) to screen for obesity. Obesity is a serious medical condition that can cause complications such as metabolic syndrome, high blood pressure, atherosclerosis, heart disease, diabetes, high serum cholesterol, cancers, and sleep disorders. Thus, there is a need to reduce obesity.

Fatty liver, or hepatic steatosis, is a term that describes the buildup of fat in the liver. Excessive alcohol use causes fat to accumulate, damages the liver, and cirrhosis may develop. Nonalcoholic fatty liver disease (NAFLD) is a fatty liver disease associated with obesity-related disorders, such as type-2 diabetes and metabolic syndrome, occurring in people who drink little or no alcohol. Nonalcoholic steatohepatitis (NASH) is a more advanced and severe subtype of NAFLD where steatosis is complicated by liver-cell injury and inflammation, with or without fibrosis. NASH can be severe and can lead to cirrhosis, in which the liver is permanently damaged and scarred and no longer able to work properly. Insulin resistance, altered lipid storage and metabolism, accumulation of cholesterol within the liver, oxidative stress resulting in increased hepatic injury, and bacterial translocation secondary to disruption of gut microbiota have all been implicated as important co-factors contributing to progression of NASH. Due to the growing epidemic of obesity and diabetes, NASH is projected to become the most common cause of advanced liver disease and the most common indication for liver transplantation.

Lee et al. report dilauroyl phosphatidylcholine (DLPC) is an LRH-1 agonist ligand in vitro. DLPC treatment induces bile acid biosynthetic enzymes in mouse liver, increases bile acid levels, and lowers hepatic triglycerides and serum glucose. DLPC treatment also decreases hepatic steatosis and improves glucose homeostasis in two mouse models of insulin resistance. Nature volume 474, pages 506-510 (2011). Sahini et al. report differentially expressed genes (DEGs) were identified which are mechanistically linked to lipid droplet (LD) formation in hepatocytes. LD-associated DEGs frequently regulated in patient samples were identified. Liver-receptor homolog-1 (NR5A2), was commonly repressed among patients examined. Translational Research, 177:41-69 (2016).

In certain examples, this disclosure relates to methods to prevent or treat hepatic steatosis or metabolic syndrome, comprising administrating compounds disclosed herein in an effective amount to a subject in need thereof. In certain examples, the subject is at risk of, exhibiting symptoms of, or diagnosed with nonalcoholic fatty liver disease (NAFLD). In certain examples, the subject is at risk of, exhibiting symptoms of, or diagnosed with nonalcoholic steatohepatitis (NASH). In certain examples, the subject is at risk of, exhibiting symptoms of, or diagnosed with alcoholic liver disease (ALD). In certain examples, the subject is at risk of, exhibiting symptoms of, or diagnosed with alcoholic steatohepatitis (ASH).

In certain examples, a subject is at risk of NAFLD due to obesity, insulin resistance, an enlarged liver, signs of cirrhosis, or abnormal levels of liver enzymes, triglycerides and/or cholesterol. Signs of insulin resistance include darkened skin patches over your knuckles, elbows, and knees. Signs of cirrhosis include jaundice, a condition that causes your skin and whites of your eyes to turn yellow. A sign of NAFLD or NASH includes blood test showing increased levels of the liver enzymes alanine aminotransferase (ALT) and aspartate aminotransferase (AST). An enlarged liver or an abnormal amount of fat in a liver may be identified by ultrasound, computerized tomography (CT) scans, magnetic resonance imaging or combinations thereof. A liver biopsy may be used to detect liver inflammation and damage to diagnose NASH.

Metabolic syndrome is typically diagnosed in the presence of three or more of the following medical issues: large waist size, e.g., 40 inches or more, high triglycerides e.g., triglyceride level of 150 mg/dL or higher, low levels of HDL cholesterol less than 50 mg/dL, high blood pressure, e.g., 130/85 mmHg or higher, and high blood glucose (or blood sugar) levels, a fasting blood sugar level of 100 mg/dL or higher.

In another aspect, the disclosure relates to methods to control or reduce the serum cholesterol level, comprising administrating compounds disclosed herein in an effective amount to a subject in need thereof. In certain examples, the subject has a borderline high serum cholesterol level, 200-239 mg/dL. In certain examples, the subject has a high serum cholesterol level, >240 mg/dL. In certain examples, the subject is at risk of, exhibiting symptoms, or diagnosed with hypercholesterolemia.

In another aspect, the disclosure relates to methods to prevent or treat hepatic steatosis, comprising administrating a compound disclosed herein in an effective amount to a subject in need thereof. In certain examples, the subject is at risk of, exhibiting symptoms of, or diagnosed with alcoholic liver disease (ALD). In certain examples, the subject is at risk of, exhibiting symptoms of, or diagnosed with alcoholic steatohepatitis (ASH). In certain examples, the subject is at risk of, exhibiting symptoms of, or diagnosed with nonalcoholic fatty liver disease (NAFLD). In certain examples, the subject is at risk of, exhibiting symptoms of, or diagnosed with nonalcoholic steatohepatitis (NASH). In certain examples, a subject is at risk of NAFLD due to obesity, insulin resistance, an enlarged liver, signs of cirrhosis, or abnormal levels of liver enzymes, triglycerides and/or cholesterol. Signs of insulin resistance include darkened skin patches over your knuckles, elbows, and knees. Signs of cirrhosis include jaundice, a condition that causes your skin and whites of your eyes to turn yellow. A sign of NAFLD or NASH includes blood test showing increased levels of the liver enzymes alanine aminotransferase (ALT) and aspartate aminotransferase (AST). An enlarged liver or an abnormal amount of fat in a liver may be identified by ultrasound, computerized tomography (CT) scans, magnetic resonance imaging or combinations thereof. A liver biopsy may be used to detect liver inflammation and damage to diagnose NASH.

The precise therapeutically effective amount of the compounds of this disclosure will depend on a number of factors. There are variables inherent to the compounds including, but not limited to, the following: molecular weight, absorption, bioavailability, distribution in the body, tissue penetration, half-life, metabolism, protein binding, and excretion. These variables determine what dose of compound needs to be administered in a sufficient percentage and for a sufficient amount of time to have the desired effect on the condition being treated (e.g., neoplasm). The duration of drug exposure will be limited only by the compound half-life, and side effects from treatment requiring cessation of dosing. The amount of compound administered will also depend on factors related to patients and disease including, but not limited to, the following: the age, weight, concomitant medications, and medical condition of the subject being treated, the precise condition requiring treatment and its severity, the nature of the formulation, and the route of administration. Ultimately the dose will be at the discretion of the attendant physician or veterinarian. Typically, the compound disclosed herein will be given for treatment in the range of 0.01 to 30 mg/kg body weight of recipient (mammal) per day or per dose or per cycle of treatment and more usually in the range of 0.1 to 10 mg/kg body weight per day or per dose or per cycle of treatment. Thus, for an adult human being treated for a condition, the actual amount per day or per dose or per cycle of treatment would usually be from 1 to 2000 mg and this amount may be given in a single or multiple doses per day or per dose or per cycle of treatment. Dosing regimens may vary significantly and will be determined and altered based on clinical experience with the compound. The full spectrum of dosing regimens may be employed ranging from continuous dosing (with daily doses) to intermittent dosing. A therapeutically effective amount of a pharmaceutically acceptable salt of a compound disclosed herein may be determined as a proportion of the therapeutically effective amount of the compound as the free base.

The methods of treating or preventing diseases or conditions associated with LRH-1 (e.g., diabetes, cancer, or cardiovascular disease) in a subject can further comprise administering to the subject one or more additional agents. The one or more additional agents and the compounds described herein or pharmaceutically acceptable salts or prodrugs thereof can be administered in any order, including concomitant, simultaneous, or sequential administration. Sequential administration can be administration in a temporally spaced order of up to several days apart. The methods can also include more than a single administration of the one or more additional agents and/or the compounds described herein or pharmaceutically acceptable salts or prodrugs thereof. The administration of the one or more additional agents and the compounds described herein or pharmaceutically acceptable salts or prodrugs thereof can be by the same or different routes and concurrently or sequentially.

Additional therapeutic agents include, but are not limited to, chemotherapeutic agents, antibodies, antivirals, steroidal and non-steroidal anti-inflammatoires, conventional immunotherapeutic agents, cytokines, chemokines, and/or growth factors. The additional therapeutic agents can be biomolecules.

A chemotherapeutic agent, chemotherapy agent, chemotherapeutic, anti-cancer agent, or the like is a compound or composition effective in inhibiting or arresting the growth of an abnormally growing cell. Such agents can be recognized to aid in the treatment of a cancer. Thus, such an agent may be used therapeutically to treat cancer as well as other diseases marked by abnormal cell growth. Illustrative examples of chemotherapeutic compounds include, but are not limited to, bexarotene, gefitinib, erlotinib, gemcitabine, paclitaxel, docetaxel, topotecan, irinotecan, temozolomide, carmustine, vinorelbine, capecitabine, leucovorin, oxaliplatin, bevacizumab, cetuximab, panitumumab, bortezomib, oblimersen, hexamethylmelamine, ifosfamide, CPT-11, deflunomide, cycloheximide, dicarbazine, asparaginase, mitotant, vinblastine sulfate, carboplatin, colchicine, etoposide, melphalan, 6-mercaptopurine, teniposide, vinblastine, antibiotic derivatives (e.g. anthracyclines such as doxorubicin, liposomal doxorubicin, and diethylstilbestrol doxorubicin, bleomycin, daunorubicin, and dactinomycin); antiestrogens (e.g., tamoxifen); antimetabolites (e.g., fluorouracil (FU), 5-FU, methotrexate, floxuridine, interferon alpha-2B, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cisplatin, vincristine and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chlorambucil, mechlorethamine (nitrogen mustard) and thiotepa); and steroids (e.g., bethamethasone sodium phosphate).

In some cases, contemplated examples of chemotherapeutic agents include the following molecules or derivatives such as temozolomide, carmustine, bevacizumab, procarbazine, lomustine, vincristine, gefitinib, erlotinib, cisplatin, carboplatin, oxaliplatin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin, mithramycin, vinblastine, vindesine, vinorelbine, paclitaxel, taxol, docetaxel, etoposide, teniposide, amsacrine, topotecan, camptothecin, bortezomib, anagrelide, tamoxifen, toremifene, raloxifene, droloxifene, idoxifene, fulvestrant, bicalutamide, flutamide, nilutamide, cyproterone, goserelin, leuprorelin, buserelin, megestrol, anastrozole, letrozole, vorozole, exemestane, finasteride, marimastat, trastuzumab, cetuximab, dasatinib, imatinib, combretastatin, thalidomide, azacitidine, azathioprine, capecitabine, chlorambucil, cyclophosphamide, cytarabine, daunorubicin, doxifluridine, epothilone, irinotecan, mechlorethamine, mercaptopurine, mitoxantrone, pemetrexed, tioguanine, valrubicin and/or lenalidomide or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

Any of the aforementioned therapeutic agents can be used in any combination with the compositions described herein. Combinations are administered either concomitantly (e.g., as an admixture), separately but simultaneously (e.g., via separate intravenous lines into the same subject), or sequentially (e.g., one of the compounds or agents is given first followed by the second). Thus, the term combination is used to refer to concomitant, simultaneous, or sequential administration of two or more agents. The term “combination with” when used to describe administration with an additional treatment means that the agent may be administered prior to, together with, or after the additional treatment, or a combination thereof.

Optionally, a compound or therapeutic agent as described herein may be administered in combination with a radiation therapy, an immunotherapy, a gene therapy, or a surgery.

The methods and compounds as described herein are useful for both prophylactic and therapeutic treatment. For prophylactic use, a therapeutically effective amount of the compounds and compositions or pharmaceutically acceptable salts thereof as described herein are administered to a subject prior to onset (e.g., before obvious signs of an LRH-1-related disease), during early onset (e.g., upon initial signs and symptoms of an LRH-1-related disease), or after the development of an LRH-1-related disease. Prophylactic administration can occur for several days to years prior to the manifestation of symptoms of an LRH-1-related disease. Therapeutic treatment involves administering to a subject a therapeutically effective amount of the compounds and compositions or pharmaceutically acceptable salts thereof as described herein after an LRH-1-related disease is diagnosed.

The compounds described herein are also useful in modulating LRH-1 in a cell. The methods for modulating LRH-1 activity in a cell includes contacting a cell with an effective amount of one or more of the compounds as described herein. Optionally, the contacting is performed in vivo. Optionally, the contacting is performed in vitro.

The methods herein for prophylactic and therapeutic treatment optionally comprise selecting a subject with or at risk of developing an LRH-1-related disease. A skilled artisan can make such a determination using, for example, a variety of prognostic and diagnostic methods, including, for example, a personal or family history of the disease or condition, clinical tests (e.g., imaging, biopsy, genetic tests), and the like. Optionally, the methods herein can be used for preventing relapse of cancer in a subject in remission (e.g., a subject that previously had cancer).

### VI. Kits

Also provided herein are kits for treating or preventing an LRH-1-related disease (e.g., diabetes, cancer, and/or cardiovascular disease) in a subject. A kit can include any of the compounds or compositions described herein. For example, a kit can include one or more compounds of Formula I and/or Formula II. A kit can further include one or more additional agents, such as one or more anti-inflammatory agents and/or chemotherapeutic agents. A kit can include an oral formulation of any of the compounds or compositions described herein. A kit can include an intravenous formulation of any of the compounds or compositions described herein. A kit can additionally include directions for use of the kit (e.g., instructions for treating a subject), a container, a means for administering the compounds or compositions (e.g., a syringe), and/or a carrier.

As used herein the terms treatment, treat, or treating refer to a method of reducing one or more symptoms of a disease or condition. Thus in the disclosed method, treatment can refer to a 10%, 20%, 30%, 40%, 50%, 60%, 70%, 80%, 90%, or 100% reduction in the severity of one or more symptoms of the disease or condition. For example, a method for treating a disease is considered to be a treatment if there is a 10% reduction in one or more symptoms or signs (e.g., size of the tumor or rate of tumor growth) of the disease in a subject as compared to a control. As used herein, control refers to the untreated condition (e.g., the tumor cells not treated with the compounds and compositions described herein). Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, the terms prevent, preventing, and prevention of a disease or disorder refer to an action, for example, administration of a composition or therapeutic agent, that occurs before or at about the same time a subject begins to show one or more symptoms of the disease or disorder, which inhibits or delays onset or severity of one or more symptoms of the disease or disorder. It is not intended that the present disclosure be limited to complete prevention. In some examples, the onset is delayed, or the severity of the disease is reduced.

As used herein, references to decreasing, reducing, or inhibiting include a change of 10%, 20%, 30%, 40%, 50%, 60%, 70%, 80%, 90% or greater as compared to a control level. Such terms can include, but do not necessarily include, complete elimination.

As used herein, subject means any animal, including mammals and non-mammals. Mammals include, for example, humans; non-human primates, e.g., apes and monkeys; cattle; horses; sheep; rats; mice; pigs; and goats. Non-mammals include, for example, fish and birds. In some examples, subject refers to a human patient, livestock, rodent, monkey, or domestic pet.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Synthesis of Hexahydropentalene Derivatives

The general strategies for preparing hexahydropentalene derivatives are illustrated in the schemes below using procedures set out in, or as appropriately modified from, Cato et al. Differential Modulation of Nuclear Receptor LRH-1 through Targeting Buried and Surface Regions of the Binding Pocket, Journal of Medicinal Chemistry, 65, 9, 6888-6902 (2022) and Whitby et al., Small molecule agonists of the orphan nuclear receptors steroidogenic factor-1 (SF-1, NR5A1) and liver receptor homologue-1 (LRH-1, NR5A2). Journal of Medicinal Chemistry, 54, 2266-2281 (2011).

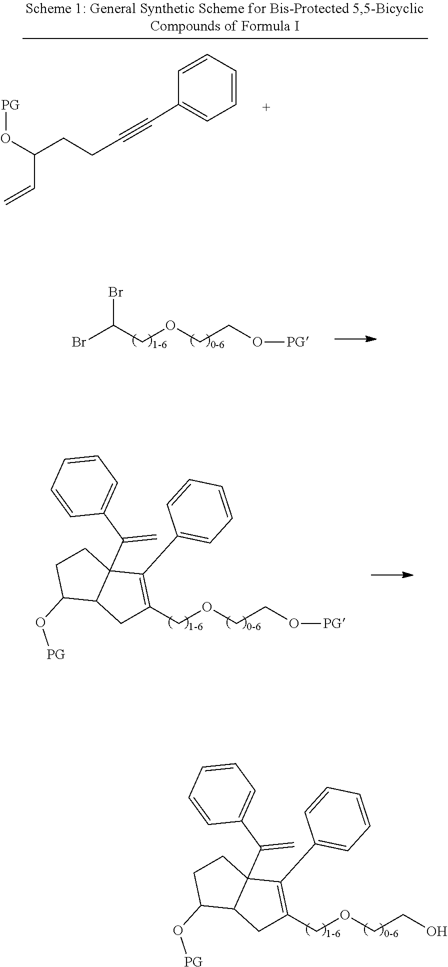

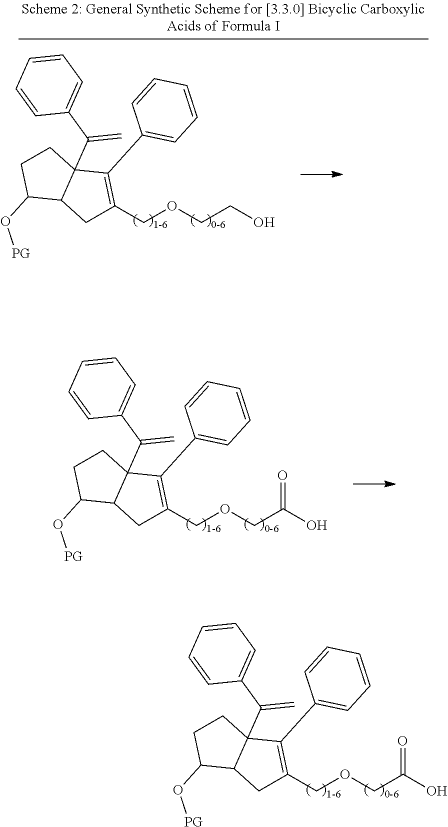

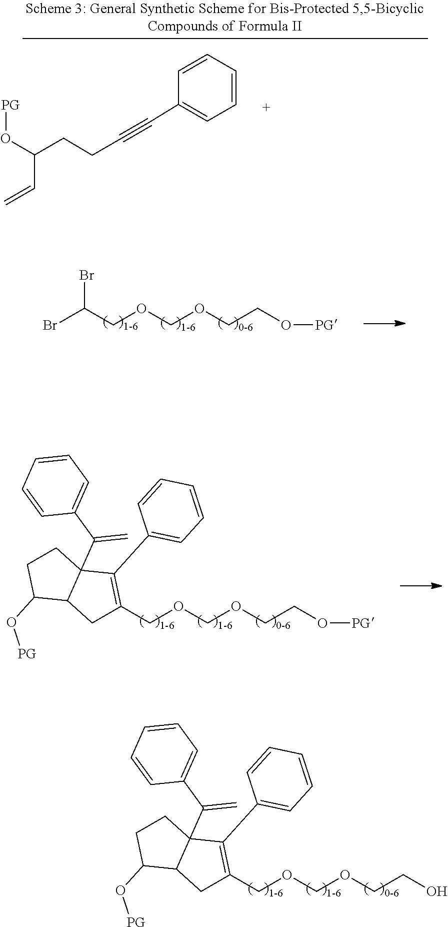

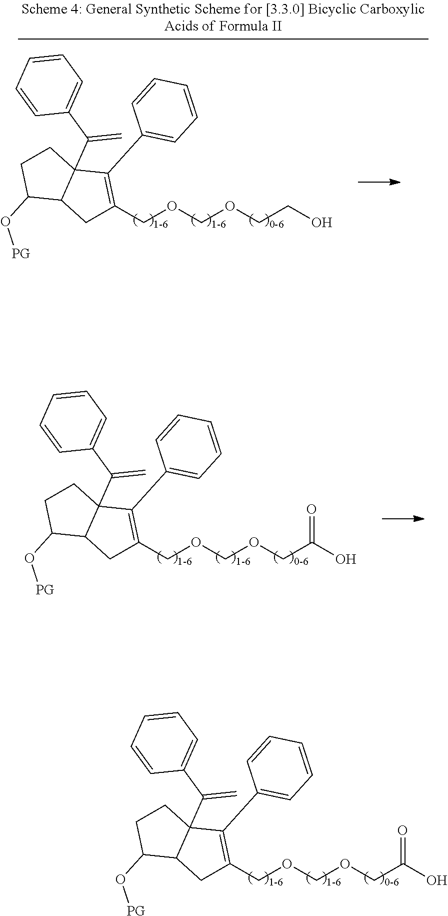

In Schemes 1-4, “PG” and “PG’” represent protecting groups, such as methyoxylmethyl (MOM), tert-butyldimethylsilyl (TBS), tert-butyldiphenylsilyl (TBDPS), and the like.

### General Cyclization and Deprotection Procedure

Bis(cyclopentadienyl) zirconium (IV) dichloride (zirconecene dichloride) (1.2 equiv.) was dried by azeotroping away latent water with benzene four times before being placed under nitrogen, dissolved in tetrahydrofuran (THF) and cooled to −78° C. in a dry ice/acetone bath. The resulting solution of zirconecene dichloride was treated with nBuLi (2.4 equiv.) to form a clear, light yellow solution and allowed to stir. After approximately 30 minutes, azeotroped tert-butyldimethyl((7-phenylhept-1-en-6-yn-3-yl)oxy) silane (19) (1.0 equiv.) in dry THF was added portionwise to afford a pink-orange solution, and the reaction mixture was held at −78° C. for 30 minutes before allowing to warm and stir at room temperature for 2.5 hours. The reaction mixture was then re-cooled to −78° C. and the required azeotroped dibromoheptane (1.1 equiv.) was added in dry THF. Freshly prepared lithium diisopropylamine (LDA, 1.0 M, 1.1 equiv.) was added at −78° C. and stirred for 15 minutes. Lithium phenylacetylide (3.6 equiv.) was then prepared and added to the reaction mixture dropwise in dry THF. The resulting dark reddish brown solution was stirred at −78° C. for 1.5 hours. The reaction was then quenched with methanol and saturated aqueous sodium bicarbonate and allowed to warm to room temperature to form a light yellow slurry. The resulting slurry was poured over water and extracted with ethyl acetate four times. The combined organic layers were washed with brine, dried with MgSO₄, and concentrated in vacuo. The resulting yellow oil was roughly purified on a plug of silica and eluted with 20% EtOAc/Hexanes to afford a yellow oil which is a mixture of phenylacetylene and desired bis-protected [3.3.0] bicyclic compounds, which was carried on without further purification. This procedure affords exo and endo diastereomers in a 1.6:1 ratio as determined by characteristic 1-H NMR signals.

The crude mixture (1.0 equiv.) was dissolved in THF, and tetrabutylammonium fluoride (1.5 equiv.) was added and the resulting dark brown solution was stirred open to air for 18 hours, concentrated, and subjected directly to silica gel chromatography in 5-10% EtOAc/Hexanes to separate both endo and exo diastereomers (with endo eluting before exo) of the desired compounds as clear, colorless oils.

### Synthesis of Representative Compounds according to Formula I and Formula II

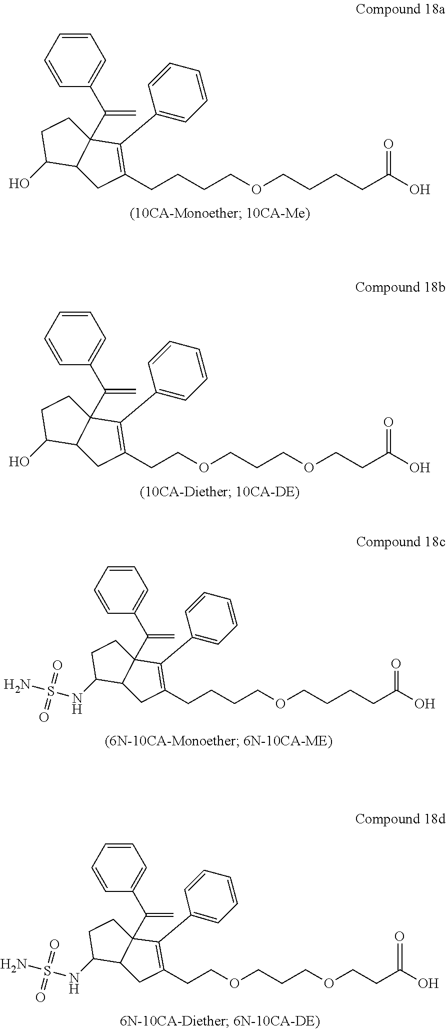 [SMILES: C=C(c1ccccc1)C12CCC(O)C1CC(CCCCOCCCCC(=O)O)=C2c1ccccc1 ; C=C(c1ccccc1)C12CCC(O)C1CC(CCOCCCOCCC(=O)O)=C2c1ccccc1 ; C=C(c1ccccc1)C12CCC(NS(N)(=O)=O)C1CC(CCCCOCCCCC(=O)O)=C2c1ccccc1 ; C=C(c1ccccc1)C12CCC(NS(N)(=O)=O)C1CC(CCOCCCOCCC(=O)O)=C2c1ccccc1]

Representative compounds according to Formula I and II, including Compounds 18a, 18b, 18c, and 18d as shown above were synthesized as outlined below. As outlined above in the general procedure, for example, a mixture of diastereomers can be obtained.

### Glycol Derivatives

6-iodohex-1-ene (1)

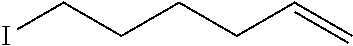

To a round bottom flask under nitrogen flow was added sodium iodide (93.68 g, 5 equivalents, 625 mmol), acetone (300 mL) and 6-bromo-1-hexene (125 mmol, 1 eq, 16.75 mL). The suspension was stirred and heated to reflux for 18 h. The reaction was then loaded onto a silica plug, eluted with hexanes, 1:1 hexanes and MTBE, and MTBE (75 mL each). The organics were concentrated to a colorless oil (21.9 g, 104 mmol, 83%). 1H NMR (600 MHZ, Chloroform-d) δ 5.76 (ddtd, J=17.0, 10.2, 6.7, 1.5 Hz, 1H), 4.99 (dp, J=17.1, 1.7 Hz, 1H), 4.94 (dp, J=10.2, 1.6 Hz, 1H), 3.17 (td, J=7.0, 1.5 Hz, 2H), 2.05 (qd, J=7.3, 1.5 Hz, 2H), 1.85-1.76 (m, 2H), 1.48 (pd, J=7.5, 1.5 Hz, 2H).

### 5-(hex-5-en-1-yloxy) pentan-1-ol (2)

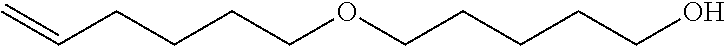

Sodium hydride (60% in mineral oil, 3.46 g, 86 mmol, 1 eq) was added to a flame dried round bottom flask under positive nitrogen pressure followed by 115 mL of DMF and 115 mL of THF. The mixture was cooled to 0° C. and then 1,5-pentane diol (27.2 mL, 3.0 eq, 260 mmol) was added dropwise. The resulting solution was stirred for 20 minutes, then 6-iodohex-1-ene (1) was added dropwise. The reaction was stirred overnight and allowed to warm to room temperature. In the morning, saturated ammonium chloride was added to quench the reaction and then the mixture was extracted with ethyl acetate (3×100 mL). The organics were combined, dried, and concentrated to provide a crude oil that was purified by silica chromatography (0-20% EtOAc:hexanes) to yield the title compound (8.01 g, 43 mmol, 50% yield). ¹H NMR (600 MHz, Chloroform-d) δ 5.80 (ddtd, J=16.9, 10.1, 6.7, 0.9 Hz, 1H), 5.00 (dtd, J=17.1, 2.6, 1.5 Hz, 1H), 4.94 (ddq, J=10.1, 2.2, 1.1 Hz, 1H), 3.64 (td, J=6.5, 0.9 Hz, 2H), 3.40 (tdd, J=6.6, 3.8, 0.9 Hz, 4H), 2.06 (tdd, J=7.8, 6.4, 1.3 Hz, 2H), 1.63-1.55 (m, 6H), 1.47-1.40 (m, 4H).

### tert-butyl ((5-(hex-5-en-1-yloxy) pentyl)oxy)diphenylsilane (3)

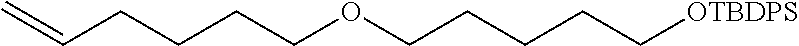

5-(Hex-5-en-1-yloxy) pentan-1-ol (2) (8.01 g, 43 mmol) was dissolved in 400 mL of dry THF, followed by 4.39 g imidazole (1.5 eq, 64.5 mmol). The mixture was stirred for 5 minutes until it was homogeneous and then TBDPS-C₁ (1.2 eq, 51.6 mmol, 13.42 mL) was added dropwise followed by overnight stirring at room temperature. The reaction was concentrated to approximately 150 mL and passed through a celite plug to remove imidazolium salts. The filtrate was then concentrated to a clear oil that was purified by silica chromatography (1-7% EtOAc/hexanes) (12.9 g, 30.37 mmol, 70% yield). 1H NMR (600 MHZ, Chloroform-d) δ 7.69-7.64 (m, 4H), 7.44-7.35 (m, 6H), 5.80 (ddt, J=16.9, 10.2, 6.7 Hz, 1H), 5.00 (dq, J=17.1, 1.7 Hz, 1H), 4.94 (ddt, J=10.2, 2.5, 1.2 Hz, 1H), 3.65 (t, J=6.5 Hz, 2H), 3.38 (dt, J=9.3, 6.6 Hz, 4H), 2.10-2.03 (m, 2H), 1.62-1.51 (m, 6H), 1.48-1.37 (m, 4H), 1.04 (s, 9H).

### 5-((5-((tert-butyldiphenylsilyl)oxy) pentyl)oxy) pentanal (4)

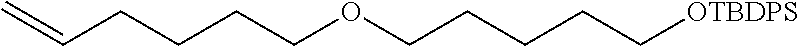

Starting material (3) (9.59 mmol; 4.06 g) was dissolved in 150 mL of dry DCM and the reaction was cooled to −78° C. Next, ozone was bubbled through the reaction while stirring until a blue color persisted. Oxygen was then bubbled through the reaction to flush the remaining ozone and then three equivalents (7.73 g) of triphenylphosphine was added. The reaction was warmed to room temperature and stirred for three hours. The solvent was removed in vacuo and the resulting oil purified by silica chromatography (0-20% EtOAc/hexanes). 3.083 g, 76%, 1H NMR (600 MHZ, Chloroform-d) δ 9.76 (t, J=1.8 Hz, 1H), 7.71-7.60 (m, 4H), 7.46-7.33 (m, 6H), 3.66 (t, J=6.5 Hz, 2H), 3.39 (dt, J=18.5, 6.5 Hz, 4H), 2.45 (td, J=7.3, 1.8 Hz, 2H), 1.75-1.67 (m, 2H), 1.64-1.51 (m, 6H), 1.45-1.36 (m, 2H), 1.04 (s, 9H).

### tert-butyl ((5-((5,5-dibromopentyl)oxy) pentyl)oxy)diphenylsilane (5)

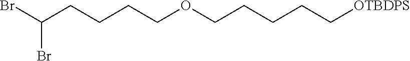

Under nitrogen, a solution of triphenylphosphite (2.35 mL, 9 mmol, 1.1 equiv) in DCM (50 mL) was cooled to −78° C. Bromine (0.48 mL, 9 mmol, 1.1 equiv) and triethylamine (1.32 mL, 9 mmol, 1.1 equiv) were sequentially added dropwise at −78° C. The reaction was stirred for 5 minutes, then 5-((5-((tert-butyldiphenylsilyl)oxy) pentyl)oxy) pentanal (4) (5.8 g, 14 mmol, 1.0 equiv) was added as a solution in DCM (10 mL) via syringe at −78° C. The reaction was stirred for 5 h and allowed to warm to ambient temperature. The whole reaction was then poured over a pad of silica. The filtrate was concentrated and purified on a short plug of silica with 5% MTBE in hexanes to afford the title compound as a clear, colorless oil (3.35 g, 72%). 1H NMR (600 MHZ, Chloroform-d) δ 7.64 (dt, J=8.0, 1.4 Hz, 4H), 7.44-7.31 (m, 6H), 5.67 (td, J=6.2, 1.1 Hz, 1H), 3.63 (td, J=6.5, 1.2 Hz, 2H), 3.42-3.29 (m, 4H), 2.38 (dddt, J=7.6, 6.1, 4.2, 2.0 Hz, 2H), 1.66-1.46 (m, 9H).

### di(1,3-dioxan-2-yl) methane (6)

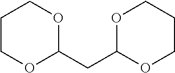 [SMILES: C1COC(CC2OCCCO2)OC1]

Compound 6 (di(1,3-dioxan-2-yl) methane) was prepared as reported in Jahromi et al., J. Med. Chem., 2013, 56, 9471.

### 3,3′-(propane-1,3-diylbis(oxy))bis(propan-1-ol) (7)

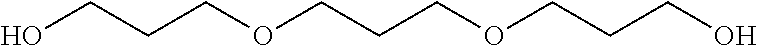 [SMILES: OCCCOCCCOCCCO]

A dry 3-neck round bottom flask charged with a stir bar was removed from a drying oven and sealed with septum stoppers before being connected to a vacuum line. The flask was allowed to cool to room temperature under vacuum. Once cool, di(1,3-dioxan-2-yl) methane (21 g, 111.85 mmol, 1.0 equiv) and freshly prepared Cu^{II} (OTf)₂ (810 mg, 2.24 mmol, 2% loading) were added to the flask under positive nitrogen pressure. The flask was resealed with a septum stopper. The atmosphere was exchanged by applying vacuum and backfilling with N₂ (this process was conducted a total of three times). Degassed tetrahydrofuran (THF, 65 ml/mmol di(1,3-dioxan-2-yl) methane) was delivered to the flask and the resulting solution was cooled to 0° C. Once cool, BH₃·DMS (37.1 ml, 391.5 mmol, 3.5 equiv) was added. The resulting mixture was allowed to stir overnight while warming to room temperature. The reaction was quenched with MeOH and subjected to a short celite plug. The resulting crude mixture was concentrated in vacuo and subjected to silica gel chromatography (5-15% MeOH/EtOAc) to afford the title compound (13.3 g, 62%).

### 2,2-dimethyl-3,3-diphenyl-4,8,12-trioxa-3-silapentadecan-15-ol (8)

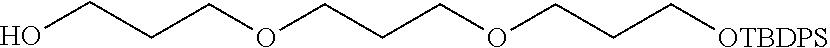

To a solution of tripropylene glycol (13.3 g, 69.2 mmol, 1.3 equiv) and imidazole (3.62 g, 53.2 mmol, 1 equiv) in anhydrous THF (800 ml) in an oven-dried flask under nitrogen at r.t. was added tert-butyl (chloro)diphenylsilane (13.85 ml, 53.2 mmol, 1 equiv) and stirred for 2.5 h. The resulting solution was concentrated in vacuo and subjected to silica gel chromatography (20-50% EtOAc/Hexanes) to afford the title compound (12.3 g, 54%). 1H NMR (600 MHZ, Chloroform-d) δ 7.67-7.65 (m, 4H), 7.43-7.35 (m, 6H), 3.77-3.73 (m, 4H), 3.61-3.58 (m, 2H), 3.54 (t, J=6.4 Hz, 2H), 3.49 (t, J=6.4 Hz, 2H), 3.46 (t, J=6.3 Hz, 2H), 2.42 (s, 1H), 1.85-1.78 (m, 6H), 1.04 (d, J=0.6 Hz, 9H). ¹³C NMR (151 MHz, Chloroform-d) δ 135.56, 133.99, 129.54, 127.60, 70.46, 68.46, 67.76, 67.63, 62.35, 60.84, 32.72, 31.96, 30.07, 26.86, 19.24.

### 2,2-dimethyl-3,3-diphenyl-4,8,12-trioxa-3-silapentadecan-15-al (9)

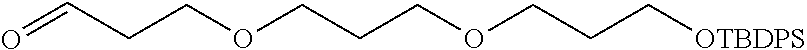

To a solution of 2,2-dimethyl-3,3-diphenyl-4,8,12-trioxa-3-silapentadecan-15-ol (12.3 g, 28.6 mmol, 1 equiv) in anhydrous THF (500 ml) in an oven-dried flask under nitrogen at 0° C. was added Dess-Martin Periodinane (14.5 g, 34.3 mmol, 1.2 equiv) and allowed to warm to room temperature and stirred for 1.5 h. The reaction mixture was then filtered through a pad of celite. The resulting solution was concentrated in vacuo and subjected to silica gel chromatography (20-50% EtOAc/Hexanes) to afford the title compound (10.5 g, 86%). ¹H NMR (600 MHZ, Chloroform-d) δ 9.77 (td, J=2.0, 0.7 Hz, 1H), 7.69-7.64 (m, 4H), 7.44-7.34 (m, 6H), 3.74 (dt, J=8.1, 6.1 Hz, 4H), 3.54 (t, J=6.4 Hz, 2H), 3.49 (t, J=6.4 Hz, 2H), 3.45 (t, J=6.3 Hz, 2H), 2.66-2.61 (m, 2H), 1.81 (dp, J=8.0, 6.4 Hz, 4H), 1.04 (s, 9H), ¹³C NMR (151 MHZ, Chloroform-d) δ 201.30, 135.56, 133.99, 129.55, 127.61, 68.25, 67.58, 64.47, 60.83, 43.87, 32.75, 29.98, 26.85, 19.24.

### 15,15-dibromo-2,2-dimethyl-3,3-diphenyl-4,8,12-trioxa-3-silapentadecane (10)

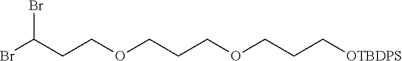

To a cold solution of triphenyl phosphite (7.1 g, 27.0 mmol, 1.1 equiv) in anhydrous dichloromethane (350 mL) maintained at −78° C. under N₂ flow, bromine (1.4 ml, 27.0 mmol, 1.1 equiv) was added dropwise. Anhydrous triethylamine (4.1 mL, 29.5 mmol, 1.2 equiv) and 2,2-dimethyl-3,3-diphenyl-4,8,12-trioxa-3-silapentadecan-15-al (10.5 g, 24.5 mmol, 1 equiv) were added to the faint orange solution. The reaction mixture was stirred for 4 h and allowed to warm to room temperature after 3 h. The resulting solution was concentrated in vacuo and subjected to silica gel chromatography (10-30% MTBE/Hexanes) to afford the title compound (10.9 g, 78%). 1H NMR (600 MHZ, Chloroform-d) ò 7.68-7.65 (m, 4H), 7.44-7.40 (m, 2H), 7.40-7.36 (m, 4H), 5.82 (t, J=6.7 Hz, 1H), 3.75 (t, J=6.1 Hz, 2H), 3.55 (t, J=6.4 Hz, 2H), 3.52-3.44 (m, 6H), 2.61 (q, J=5.9 Hz, 2H), 1.86-1.77 (m, 4H), 1.05 (s, 9H). ¹³C NMR (151 MHz, Chloroform-d) δ 135.57, 133.98, 129.55, 128.35, 127.61, 77.23, 77.02, 76.81, 68.18, 68.05, 67.64, 67.58, 60.84, 45.59, 43.09, 32.76, 29.99, 27.25, 20.21.

Cyclization procedure for hexahydropentalene formation

Prior to cyclization, all non-volatile reagents were dried by azeotropic removal of water using benzene. A dry round bottom flask containing bis(cyclopentadienyl) zirconium (IV) dichloride (1.2 equiv) under nitrogen was dissolved in anhydrous, degassed tetrahydrofuran (THF, 8 mL/mmol enyne) and cooled to −78° C. The resulting mixture was treated with n-BuLi (2.4 equiv) and the light-yellow solution was stirred for 45 minutes. A solution of 11 ((5-(methoxymethoxy) hept-6-en-1-yn-1-yl)benzene) or 12 (tertbutyldimethyl ((7-phenylhept-1-en-6-yn-3-yl)oxy) silane) (1.0 equiv) in anhydrous, degassed THF (8 mL/mmol) was added. The resulting salmon-colored mixture was stirred at −78° C. for 45 minutes before the cooling bath was removed. The reaction mixture continued to stir at room temperature for an additional 2.5 hours. The reaction mixture was then cooled to −78° C. and the required 1,1-dibromoether tail ((5) or (10)) (1.1 equiv) was added as a solution in anhydrous THF (8 mL/mmol enyne) followed by freshly prepared lithium diisopropylamide (LDA, 1.0 M, 1.1 equiv.). After 15 minutes, a freshly prepared solution of lithium phenylacetylide (3.6 equiv) in anhydrous THF (8 mL/mmol enyne) was added dropwise and the resulting rust-colored solution was stirred at −78° C. for 1.5 hours. The reaction was quenched with methanol and saturated aqueous sodium bicarbonate slowly warming to room temperature, affording a light-yellow slurry. The slurry was poured onto water and extracted with ethyl acetate four times. The combined organic layers were washed with brine, dried with Na₂SO₄, and concentrated in vacuo. The resulting yellow oil was passed through a short plug of silica (100% EtOAc eluent) and concentrated. The crude product was dissolved in THF and treated with either HCl or TBAF. The resulting solution stirred at room temperature for 16 h. The reaction mixture was concentrated and the diastereomers were purified and separated by flash chromatography.

### 5-(4-(−6-(methoxymethoxy)-3-phenyl-3a-(1-phenylvinyl)-1,3a,4,5,6,6a-hexahydropentalen-2-yl) butoxy) pentan-1-ol (13a)

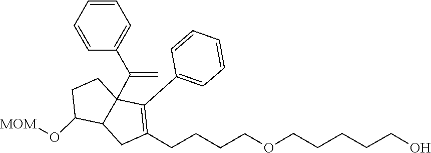

According to the general procedure, 5 (2.50 g, 4.4 mmol) was reacted with 11 (1.20 g, 4 mmol) before being treated with 5 equivalents of tetrabutylammonium fluoride to give the title compound (1.2 g, 5:1 dr, 60% yield over 2 steps) as a yellow oil after purification by flash chromatography (0-50% EtOAc/Hex eluent). 1H NMR (600 MHZ, Chloroform-d) δ 7.32-7.25 (m, 3H), 7.24-7.20 (m, 5H), 7.18 (dt, J=8.1, 1.5 Hz, 2H), 5.02 (q, J=2.5, 2.0 Hz, 1H), 4.98 (q, J=2.4, 1.9 Hz, 1H), 4.60-4.54 (m, 2H), 3.79-3.73 (m, 1H), 3.66-3.57 (m, 4H), 3.34 (td, J=6.6, 1.3 Hz, 2H), 3.28 (d, J=1.3 Hz, 3H), 2.39 (dq, J=9.6, 1.8 Hz, 1H), 2.26 (dddd, J=9.8, 7.0, 3.1, 1.2 Hz, 1H), 2.03-1.96 (m, 3H), 1.60-1.52 (m, 8H), 1.49-1.34 (m, 8H). LRMS (APCI) m/z calc'd for C₃₃H₄₄O₄: 505.3, found 473.3 (13a-CH₃O′)

### 3-(3-(2-(−6-(methoxymethoxy)-3-phenyl-3a-(1-phenylvinyl)-1,3a,4,5,6,6a-hexahydropentalen-2-yl) ethoxy) propoxy) propan-1-ol (13b)

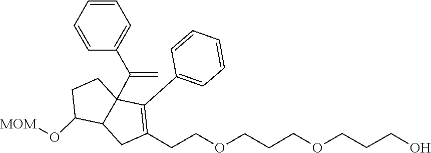

According to the general cyclization procedure, 10 (2.50 g, 4.4 mmol) was reacted with 11 (1.20 g, 4 mmol) before being treated with 5 equivalents of tetrabutylammonium fluoride to give the title compound (1.24 g, 7:1 dr, 61% yield over 2 steps) as a yellow oil after purification by flash chromatography (0-50% EtOAc/Hex eluent). 1H NMR: 1H NMR (600 MHz, Chloroform-d) § 7.32 (ddq, J=6.0, 2.3, 1.3 Hz, 2H), 7.28-7.25 (m, 2H), 7.26-7.19 (m, 6H), 5.03 (q, J=1.3 Hz, 1H), 4.98 (dd, J=2.0, 1.3 Hz, 1H), 4.58-4.53 (m, 2H), 3.76-3.75 (m, 1H), 3.58-3.54 (m, 2H), 3.48-3.44 (m, 2H), 3.42-3.35 (m, 4H), 3.27 (d, J=2.0 Hz, 3H), 2.40 (ddt, J=11.0, 7.1, 1.7 Hz, 2H), 2.36-2.25 (m, 3H), 2.07 (d, J=16.9 Hz, 1H), 1.78 (dddt, J=7.8, 5.9, 3.4, 1.8 Hz, 5H), 1.67-1.58 (m, 2H). LRMS (APCI) m/z [M+H]⁺ calc'd for C₃₂H₄₂O₅: 507.3, found 475.3 (13b-CH₃O′)

### 5-(4-((5-hydroxypentyl)oxy)butyl)-4-phenyl-3a-(1-phenylvinyl)-1,2,3,3a,6,6a-hexahydropentalen-1-ol (13c)

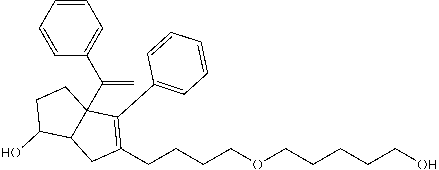 [SMILES: C=C(c1ccccc1)C12CCC(O)C1CC(CCCCOCCCCCO)=C2c1ccccc1]

According to the general procedure, 5 (2.50 g, 4.4 mmol) was reacted with 11 (1.20 g, 4 mmol) before being treated with three drops of HCl to give the title compound (1.21 g, 66% yield over 2 steps) as a yellow oil after purification by flash chromatography (0-65% EtO Ac/Hex eluent). 1H NMR (600 MHZ, Chloroform-d) δ 7.35-7.30 (m, 5H), 7.25 (m, 2H), 7.20 (t, J=6.8 Hz, 3H), 5.07 (d, J=1.6 Hz, 1H), 4.99 (dp, J=1.5 Hz, 1H), 3.95 (m, 1H), 3.65 (t, J=6.6 Hz, 3H), 3.38 (t, J=6.4 Hz, 4H), 3.33 (t, J=6.3 Hz, 2H), 2.64 (d, J=17.3 Hz, 1H), 2.47 (t, J=8.7 Hz, 1H), 2.42-2.24 (m, 2H), 2.14-1.94 (m, 6H), 1.76-1.64 (m, 4H), 1.59 (dd, J=10.7, 4.6 Hz, 4H), 1.55-1.46 (m, 1H). LRMS (APCI) m/z [M+H]⁺ calc'd for C₃₁H₄₁O₃: 461.7, found 461.3

### 5-(2-(3-(3-hydroxypropoxy) propoxy)ethyl)-4-phenyl-3a-(1-phenylvinyl)-1,2,3,3a,6,6a-hexahydropentalen-1-ol (13d)

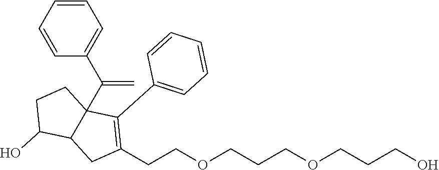 [SMILES: C=C(c1ccccc1)C12CCC(O)C1CC(CCOCCCOCCCO)=C2c1ccccc1]

According to the cyclization general procedure, 8 (2.52 g, 4.4 mmol) was reacted with 12 (1.20 g, 4 mmol) before being treated with three drops of HCl to give the title compound (758.7 g, 41% yield over 2 steps) as a yellow oil after purification by flash chromatography (10-100% EtOAc/Hex eluent). 1H NMR (600 MHZ, Chloroform-d) δ 7.36-7.31 (m, 4H), 7.30-7.24 (m, 3H), 7.22-7.20 (m, 3H), 5.05 (d, J=1.1 Hz, 1H), 4.97 (d, J=1.0 Hz, 1H), 3.92 (m, 1H), 3.73 (t, J=5.5 Hz, 4H), 3.57 (q, J=5.9, 4.7 Hz, 4H), 3.48 (dt, J=16.8, 6.0 Hz, 3H), 3.42-3.33 (m, 4H), 2.57 (td, J=10.7, 9.6, 5.9 Hz, 2H), 2.42 (t, J=8.3 Hz, 1H), 2.39-2.20 (m, 4H), 2.14-2.03 (m, 4H), 1.96-1.88 (m, 1H), 1.67-1.58 (m, 1H). LRMS (APCI) m/z [M+H]⁺ calc'd for C₃₀H₃₉O₄: 463.6, found 463.3

### Oxidation Method

To a solution of 13a-d (1 equiv) in acetonitrile (0.1 M) was added tetrapropylammonium perruthenate (TPAP) (0.1 equiv), N-methylmorpholine N-oxide (NMO) (10 equiv), and water (10 equiv) and stirred at room temperature overnight. The reaction solution was then filtered through a pad of silica with (100% EtOAc) and concentrated. The resulting mixture was purified by flash chromatography to give the title compound.

### 5-(4-(6-(methoxymethoxy)-3-phenyl-3a-(1-phenylvinyl)-1,3a,4,5,6,6a-hexahydropentalen-2-yl) butoxy) pentanoic acid (14a)

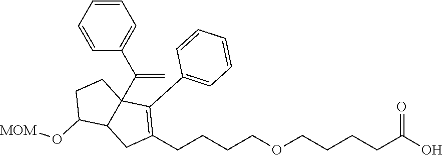

A solution of the required alcohol (504 mg, 1 eq) in 50 mL acetonitrile was treated with tetrapropylammonium perruthenate (TPAP, 0.1 equiv, 17.55 mg), N-Methylmorpholine-N-Oxide (NMO, 10 equiv, 1.17 g), and water (10 equiv, 180 microliters). The reaction mixture was allowed to stir overnight at room temperature. The reaction mixture was passed through celite, concentrated and purified on silica (0-65% EtOAc (containing 0.1% AcOH)/hexanes eluent) to provide the title compound. 70 mg, 13% 1H NMR (600 MHZ, Chloroform-d) δ 7.38-7.28 (m, 4H), 7.28-7.25 (m, 3H), 7.25-7.22 (m, 2H), 5.06 (d, J=1.2 Hz, 1H), 5.04 (t, J=1.2 Hz, 1H), 4.64 (s, 2H), 3.81 (dd, J=4.5, 2.6 Hz, 1H), 3.42 (q, J=5.8 Hz, 2H), 3.37 (dd, J=6.1, 1.2 Hz, 5H), 2.39 (t, J=7.6 Hz, 2H), 1.71-1.59 (m, 4H), 1.56-1.41 (m, 5H), 1.36-1.23 (m, 10H). LRMS (APCI) m/z calc'd for C₃₃H₄₂O₅: 519.3, found 487.3 (14a-CH₃O′)

### 3-(3-(2-(6-(methoxymethoxy)-3-phenyl-3a-(1-phenylvinyl)-1,3a,4,5,6,6a-hexahydropentalen-2-yl) ethoxy) propoxy) propanoic acid (14b)

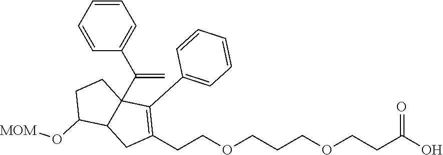

A solution of the required alcohol (504 mg, 1 eq) in 50 mL acetonitrile was treated with tetrapropylammonium perruthenate (TPAP, 0.1 equiv, 17.55 mg), N-Methylmorpholine-N-Oxide (NMO, 10 equiv, 1.17 g), and water (10 equiv, 180 microliters). The reaction mixture was allowed to stir overnight at room temperature. The reaction mixture was passed through celite, concentrated and purified on silica (20-80% EtOAc (containing 0.1% AcOH)/hexanes eluent) to provide the title compound. 285 mg, 45%, 1H NMR (600 MHZ, Chloroform-d) δ 7.35-7.30 (m, 2H), 7.29-7.25 (m, 2H), 7.24-7.20 (m, 6H), 5.03 (d, J=1.4 Hz, 1H), 4.98 (d, J=1.4 Hz, 1H), 4.61-4.55 (m, 2H), 3.80-3.74 (m, 1H), 3.67-3.63 (m, 2H), 3.52-3.46 (m, 2H), 3.42-3.34 (m, 5H), 3.29 (t, J=1.0 Hz, 2H), 2.57 (td, J=6.2, 1.4 Hz, 2H), 2.39 (dt, J=9.3, 1.6 Hz, 1H), 2.35-2.29 (m, 2H), 2.26 (dt, J=13.8, 6.5 Hz, 1H), 2.11-2.08 (m, 1H), 2.08-2.04 (m, 2H), 2.01-1.96 (m, 1H), 1.78 (pd, J=6.4, 1.6 Hz, 2H), 1.74-1.67 (m, 1H), 1.67-1.58 (m, 2H). LRMS (APCI) m/z calc'd for C₃₂H₄₀O₆: 521.3, found 489.3 (14a-CH₃O)

### 5-(4-(6-oxo-3-phenyl-3a-(1-phenylvinyl)-1,3a,4,5,6,6a-hexahydropentalen-2-yl) butoxy) pentanoic acid (14c)

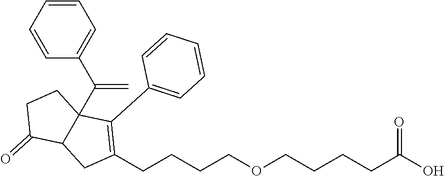 [SMILES: C=C(c1ccccc1)C12CCC(=O)C1CC(CCCCOCCCCC(=O)O)=C2c1ccccc1]

According to the oxidation general procedure, 13c (1.21 g, 2.63 mmol) was reacted with TPAP (92.4 mg, 0.26 mmol), NMO (3.07 g, 26 mmol), water (473 μL, 473 mg, 26 mmol), and acetonitrile (40 mL) to give the title compound (529 mg, 43% yield) as an oil after purification by flash chromatography (0-65% EtOAc/Hex). 1H NMR (600 MHZ, Chloroform-d) δ 7.36-7.31 (m, 2H), 7.30-7.26 (m, 3H), 7.23 (m, 3H), 7.20-7.16 (m, 2H), 5.19 (d, J=1.7 Hz, 1H), 5.08 (d, J=1.6 Hz, 1H), 3.34 (t, J=6.2 Hz, 2H), 3.27 (t, J=6.5 Hz, 2H), 2.44 (d, J=7.7 Hz, 1H), 2.35 (td, J=7.4, 1.6 Hz, 2H), 2.28 (dd, J=13.7, 9.0 Hz, 2H), 2.14-2.04 (m, 4H), 2.02-1.96 (m, 2H), 1.87 (dd, J=16.5, 7.8 Hz, 1H), 1.67 (p, J=7.4 Hz, 2H), 1.57 (dq, J=12.4, 6.6 Hz, 2H), 1.40 (h, J=6.5 Hz, 2H), 1.29 (p, J=7.7 Hz, 2H). LRMS (APCI) m/z [M+H]⁺ calc'd for C₃₁H₃₇O₄: 473.6, found 473.3

### 3-(3-(2-(6-oxo-3-phenyl-3a-(1-phenylvinyl)-1,3a,4,5,6,6a-hexahydropentalen-2-yl) ethoxy) propoxy) propanoic acid (14d)

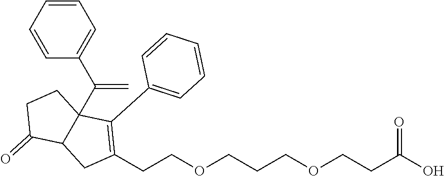 [SMILES: C=C(c1ccccc1)C12CCC(=O)C1CC(CCOCCCOCCC(=O)O)=C2c1ccccc1]

According to the oxidation general procedure, 13d (1.51 g, 3.2 mmol) was reacted with TPAP (112.5 mg, 0.32 mmol), NMO (3.74 g, 32 mmol), water (576 μL, 576 mg, 32 mmol), and acetonitrile (53 mL) to give the title compound (817 mg, 53% yield) as an oil after purification by flash chromatography (20-50% EtOAc/Hex). 1H NMR (600 MHZ, Chloroform-d) § 7.36 (d, J=4.7 Hz, 4H), 7.28 (m, 4H), 7.24-7.20 (m, 2H), 5.25 (d, J=1.5 Hz, 1H), 5.13 (d, J=1.5 Hz, 1H), 3.69 (t, J=6.1 Hz, 2H), 3.56-3.44 (m, 2H), 3.44-3.28 (m, 4H), 2.60 (t, J=6.0 Hz, 2H), 2.47 (t, J=7.8 Hz, 2H), 2.44-2.20 (m, 4H), 2.15-2.07 (m, 2H), 1.97-1.88 (m, 1H), 1.77 (dq, J=12.2, 6.2 Hz, 2H).

### Methylation Method

To a solution of 14c-d (1 equiv) in methanol (0.1 M) was added three drops of concentrated HCl and stirred at room temperature overnight. The reaction solution was then concentrated in vacuo and filtered through a pad of silica to collect the title compound.

### Methyl-5-(4-(6-oxo-3-phenyl-3a-(1-phenylvinyl)-1,3a,4,5,6,6a-hexahydropentalen-2-yl) butoxy) pentanoate (15a)

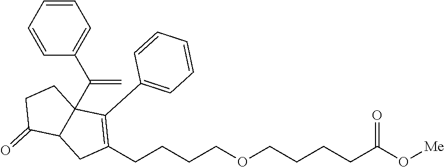 [SMILES: C=C(c1ccccc1)C12CCC(=O)C1CC(CCCCOCCCCC(=O)OC)=C2c1ccccc1]

According to the methylation general procedure, 14c (529 mg, 1.1 mmol) was reacted with HCl in methanol to give the title compound (545 mg, quantitative yield) as a yellow oil. 1H NMR (600 MHZ, Chloroform-d) δ 7.39-7.33 (m, 2H), 7.33-7.28 (m, 3H), 7.26 (t, J=3.9 Hz, 3H), 7.22-7.19 (m, 2H), 5.22 (d, J=1.5 Hz, 1H), 5.11 (d, J=1.4 Hz, 1H), 3.66 (s, 3H), 3.34 (t, J=6.4 Hz, 2H), 3.28 (t, J=6.5 Hz, 2H), 2.46 (d, J=7.8 Hz, 1H), 2.38-2.24 (m, 4H), 2.14-1.99 (m, 5H), 1.91 (dd, J=16.5, 7.8 Hz, 1H), 1.66 (ddd, J=12.4, 8.8, 6.0 Hz, 2H), 1.56 (dq, J=10.3, 6.6 Hz, 2H), 1.41 (dt, J=9.8, 6.5 Hz, 2H), 1.31 (q, J=7.8 Hz, 2H).

### Methyl-3-(3-(2-(6-oxo-3-phenyl-3a-(1-phenylvinyl)-1,3a,4,5,6,6a-hexahydropentalen-2-yl) ethoxy) propoxy) propanoate (15b)

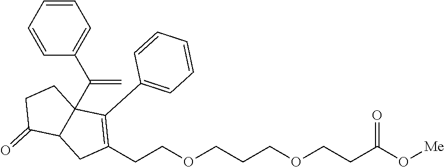 [SMILES: C=C(c1ccccc1)C12CCC(=O)C1CC(CCOCCCOCCC(=O)OC)=C2c1ccccc1]

According to the methylation general procedure, 14d (403.4 mg, 0.85 mmol) was reacted with HCl in methanol to give the title compound (299 mg, 70% yield) as a yellow oil after purification by flash chromatography (30% EtOAc/Hex). 1H NMR (600 MHZ, Chloroform-d) δ 7.36 (d, J=4.3 Hz, 4H), 7.32 (p, J=4.2 Hz, 2H), 7.28 (d, J=6.5 Hz, 2H), 7.23 (dd, J=7.7, 2.0 Hz, 2H), 5.24 (d, J=1.4 Hz, 1H), 5.12 (d, J=1.5 Hz, 1H), 3.68 (s, 3H), 3.66 (d, J=6.5 Hz, 2H), 3.46 (td, J=6.4, 3.3 Hz, 2H), 3.41-3.28 (m, 4H), 2.56 (t, J=6.4 Hz, 2H), 2.46 (d, J=7.8 Hz, 1H), 2.39 (dd, J=14.2, 7.1 Hz, 1H), 2.35 (d, J=16.4 Hz, 1H), 2.32-2.24 (m, 2H), 2.16-2.00 (m, 3H), 1.96 (dd, J=16.5, 7.9 Hz, 1H), 1.76 (p, J=6.4 Hz, 2H), 1.26 (d, J=2.6 Hz, 1H). LRMS (APCI) m/z [M+H]⁺ calc'd for C₃₁H₃₇O₅: 489.6, found 489.3

### Reductive Amination

To a flame-dried screw top test tube charged with a stir bar backfilled (3×) was added 15a-b (1.0 equiv.) and ethanol (0.1 M). Ammonia (7 M in methanol, 5.0 or 20.0 equiv) then titanium (IV) isopropoxide (0.33 mL, 1.08 mmol, 1.5 equiv) were added via syringe and stirred at room temperature for 6 hours. The test tube cap was then removed and sodium borohydride (3.0 equiv) was added portion-wise. The resulting solution was stirred at room temperature for 30 minutes before being diluted with EtOAc. The solution was adjusted to a pH of 1 with IM HCl. The layers were separated and the aqueous layer was extracted 3× with EtOAc. The combined organic layers were then dried over Na₂SO₄, filtered, and concentrated in vacuo before being purified by flash chromatography to give the title compound.

### Methyl-5-(4-(6-amino-3-phenyl-3a-(1-phenylvinyl)-1,3a,4,5,6,6a-hexahydropentalen-2-yl) butoxy) pentanoate (16a)

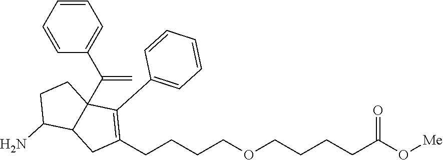 [SMILES: C=C(c1ccccc1)C12CCC(N)C1CC(CCCCOCCCCC(=O)OC)=C2c1ccccc1]

According to the reductive amination general procedure 15a (545 mg, 1.13 mmol) was reacted with ammonia (816 μL, 5.65 mmol), Ti(OiPr)₄ (517 μL, 1.7 mmol), and NaBH₄ (128 mg, 3.4 mmol) in ethanol (11 mL) to give the title compound (235 mg, 42% yield) as a clear oil after purification by flash chromatography (0-10% MeOH/DCM). 1H NMR (600 MHZ, Chloroform-d) δ 7.29-7.24 (m, 7H), 7.24-7.21 (m, 3H), 5.09 (d, J=1.4 Hz, 1H), 4.97 (d, J=1.5 Hz, 1H), 3.65 (s, 3H), 3.50 (td, J=10.4, 9.2, 4.0 Hz, 1H), 3.30 (t, J=6.4 Hz, 2H), 3.23 (q, J=5.9 Hz, 2H), 2.67 (t, J=9.1 Hz, 1H), 2.42 (d, J=17.8 Hz, 1H), 2.31 (t, J=7.5 Hz, 2H), 2.25 (dd, J=17.8, 8.9 Hz, 1H), 2.17 (q, J=7.4, 5.6 Hz, 2H), 2.07-2.00 (m, 1H), 1.75 (dt, J=12.6, 5.6 Hz, 1H), 1.72-1.61 (m, 4H), 1.54 (dt, J=9.4, 6.5 Hz, 2H), 1.38 (ddt, J=23.5, 16.8, 8.7 Hz, 4H). LRMS (APCI) m/z [M+H]⁺ calc'd for C₃₂H₄₂NO₃: 488.7, found 488.3

### Methyl-3-(3-(2-(6-amino-3-phenyl-3a-(1-phenylvinyl)-1,3a,4,5,6,6a-hexahydropentalen-2-yl) ethoxy) propoxy) propanoate (16b)

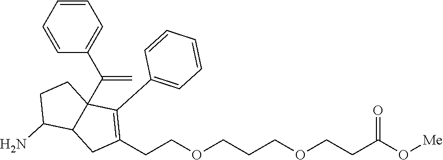 [SMILES: C=C(c1ccccc1)C12CCC(N)C1CC(CCOCCCOCCC(=O)OC)=C2c1ccccc1]

According to the reductive amination general procedure 15b (299.1 mg, 0.6 mmol) was reacted with ammonia (1.7 mL, 12.2 mmol), Ti(OiPr)₄ (272 μL, 261.4 mg, 0.92 mmol), and NaBH₄ (68.1 mg, 1.8 mmol) in ethanol (6 mL) to give the title compound (210.8 mg, 67% yield) as an orange oil after purification by flash chromatography (0-10% Methanol/DCM). 1H NMR (600 MHz, Chloroform-d) δ 7.53-7.36 (m, 2H), 7.31 (dd, J=8.3, 6.5 Hz, 3H), 7.29-7.25 (m, 3H), 7.19 (dq, J=5.3, 2.9, 2.5 Hz, 2H), 5.18 (d, J=1.5 Hz, 1H), 5.07 (d, J=1.6 Hz, 1H), 3.93-3.86 (m, 1H), 3.75-3.66 (m, 3H), 3.65 (s, 3H), 3.60-3.51 (m, 3H), 3.48 (ddd, J=9.2, 5.6, 3.2 Hz, 1H), 2.89-2.75 (m, 1H), 2.70 (dd, J=9.7, 6.7 Hz, 1H), 2.66-2.58 (m, 2H), 2.54 (ddd, J=16.2, 6.8, 4.4 Hz, 2H), 2.24-2.16 (m, 1H), 2.00-1.94 (m, 3H), 1.93-1.79 (m, 2H), 1.73 (dq, J=15.9, 6.0 Hz, 2H), 1.49-1.38 (m, 1H). LRMS (APCI) m/z [M+H]⁺ calc'd for C₃₁H₄₀NO₄: 490.6, found 490.3

### Sulfamidation Procedure

To a solution of tert-butyl alcohol (5.5 equiv) in anhydrous DCM (5 mL) in an oven-dried flask backfilled 3× with nitrogen at 0° C. was added neat chlorosulfonylisocyanate (5.0 equiv) and stirred for 30 minutes. The reaction mixture was taken out of the ice bath and allowed to stir at room temperature for an additional 90 minutes. 1 mL of the resulting solution was then added via syringe to a solution of 16a-b (1.0 equiv) and triethylamine (2.5 equiv.) in anhydrous DCM (0.1 M) under nitrogen in an oven-dried flask at 0° C. The reaction was then stirred and warmed to room temperature over 3 hours before diluting with DCM and washing with 2×10 mL 0.5 M aqueous HCl, 10 mL water and 10 mL brine. The organic layer was then dried over Na₂SO₄, filtered, and concentrated in vacuo to give crude material. This material was subjected to silica gel chromatography (10-40% EtOAc/hexanes) to collect material taken crude to the next step.

### Methyl-5-(4-(6-((N-(tert-butoxycarbonyl) sulfamoyl)amino)-3-phenyl-3a-(1-phenylvinyl)-1,3a,4,5,6,6a-hexahydropentalen-2-yl) butoxy) pentanoate (17a)

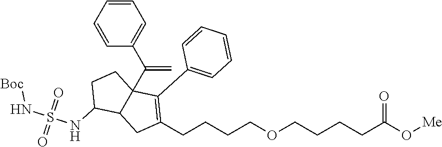 [SMILES: C=C(c1ccccc1)C12CCC(NS(=O)(=O)NC(=O)OC(C)(C)C)C1CC(CCCCOCCCCC(=O)OC)=C2c1ccccc1]

According to the general sulfamidation procedure, 16a (135 mg, 0.26 mmol) was reacted with chlorosulfonylisocyanate (113 μL, 183.9 mg, 1.3 mmol), t-BuOH (137 μL, 106.0 mg, 1.43 mmol), and TEA (90 μL, 0.65 mmol) in DCM (2.6 mL) to give the title compound (139.4 mg, 80% yield) as a clear oil after flash chromatography (20-50% EtOAc/Hexanes). 1H NMR (500 MHZ, Chloroform-d) δ 7.34-7.29 (m, 5H), 7.28-7.25 (m, 3H), 7.21 (d, J=1.8 Hz, 1H), 7.20 (t, J=1.6 Hz, 1H), 5.32 (d, J=7.9 Hz, 1H), 5.11 (d, J=1.3 Hz, 1H), 4.98 (d, J=1.3 Hz, 1H), 3.76 (ddd, J=11.0, 5.5, 2.5 Hz, 1H), 3.68 (s, 3H), 3.40 (t, J=6.3 Hz, 2H), 3.35 (t, J=6.1 Hz, 2H), 2.61 (td, J=8.9, 2.0 Hz, 1H), 2.48 (dd, J=17.7, 2.1 Hz, 1H), 2.36 (t, J=7.5 Hz, 2H), 2.24-2.08 (m, 3H), 1.97-1.85 (m, 1H), 1.79-1.66 (m, 5H), 1.60 (dq, J=9.0, 6.1 Hz, 2H), 1.56-1.48 (m, 4H), 1.46 (s, 9H). LRMS (APCI) m/z [M+H]⁺ calc'd for C₃₇H₅₁N₂O₇S: 667.8, found 610.1 (17a-t-Bu)

### Methyl-3-(3-(2-(6-((N-(tert-butoxycarbonyl) sulfamoyl)amino)-3-phenyl-3a-(1-phenylvinyl)-1,3a,4,5,6,6a-hexahydropentalen-2-yl) ethoxy) propoxy) propanoate (17b)

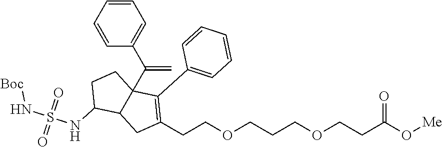 [SMILES: C=C(c1ccccc1)C12CCC(NS(=O)(=O)NC(=O)OC(C)(C)C)C1CC(CCOCCCOCCC(=O)OC)=C2c1ccccc1]

According to the general sulfamidation procedure, 16b (52.6 mg, 0.1 mmol) was reacted with chlorosulfonylisocyanate (43.5 μL, 70.7 mg, 0.5 mmol), t-BuOH (52.6 μL, 40.8 mg, 0.55 mmol), and TEA (34.8 μL, 0.25 mmol) in DCM (1 mL) to give the title compound (37.5 mg, 56% yield) as a clear oil after flash chromatography (50% EtOAc/Hexanes). 1H NMR (500 MHz, Chloroform-d) δ 7.39-7.35 (m, 2H), 7.33-7.29 (m, 2H), 7.27 (dt, J=5.9, 1.5 Hz, 3H), 7.26-7.23 (m, 3H), 5.64 (d, J=8.9 Hz, 1H), 5.09 (d, J=1.5 Hz, 1H), 5.08 (d, J=1.5 Hz, 1H), 3.85 (dtd, J=10.4, 9.2, 7.2 Hz, 1H), 3.71 (t, J=6.4 Hz, 2H), 3.69 (s, 3H), 3.68-3.63 (m, 2H), 3.60 (dt, J=9.4, 6.4 Hz, 1H), 3.55 (t, J=6.3 Hz, 2H), 3.53-3.45 (m, 2H), 2.59 (t, J=6.4 Hz, 2H), 2.57-2.47 (m, 3H), 2.08 (dt, J=14.4, 5.0 Hz, 1H), 1.98-1.85 (m, 4H), 1.67-1.60 (m, 3H), 1.55-1.49 (m, 1H), 1.43 (s, 9H), 1.41-1.31 (m, 1H). LRMS (APCI) m/z [M+H]⁺ calc'd for C₃₆H₄₉N₂O₈S: 669.8, found 686.3 (17b+H₂O)

### Deprotection Procedure

A reaction vial was charged with a stir bar and 14a-b or 17a-b (160 mg, 0.24 mmol) dissolved in dioxane and a few drops of concentrated HCl was then added. The reaction was gradually heated to 40° C. before being diluted with EtOAc and washed with 3×5 mL 0.5 M aqueous HCl, 5 ml water, and 5 mL brine. The organic layer was then dried over Na₂SO₄, filtered, and concentrated in vacuo to give the title compound after flash chromatography.

### 5-(4-(6-hydroxy-3-phenyl-3a-(1-phenylvinyl)-1,3a,4,5,6,6a-hexahydropentalen-2-yl) butoxy) pentanoic acid (18a; 10CA-Monoether; 10CA-ME)

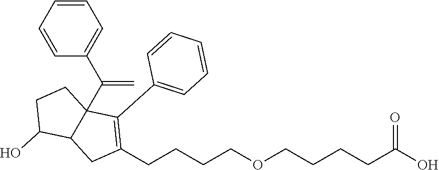 [SMILES: C=C(c1ccccc1)C12CCC(O)C1CC(CCCCOCCCCC(=O)O)=C2c1ccccc1]

Compound 14a (70 mg) was dissolved in 200 mL DCM and then 5 drops of TFA was added. The reaction was stirred at room temp until complete by TLC and HPLC. The reaction was then concentrated and purified by silica chromatography (30-100% EtOAc/Hexanes w/1% AcOH). 27 mg, 42% 1H NMR: 1H NMR (600 MHZ, Chloroform-d) δ 7.35-7.28 (m, 5H), 7.24-7.18 (m, 5H), 5.06 (d, J=1.3 Hz, 1H), 5.00 (d, J=1.4 Hz, 1H), 3.95 (s, 1H), 3.39 (t, J=6.2 Hz, 2H), 3.34 (t, J=6.3 Hz, 2H), 2.39 (t, J=7.3 Hz, 2H), 2.34-2.28 (m, 1H), 2.12-1.98 (m, 4H), 1.77-1.57 (m, 11H), 1.52-1.45 (m, 1H), 1.42 (q, J=7.6, 7.2 Hz, 1H). LRMS (APCI) m/z [M+H]⁺ calc'd for C₃₁H₃₈O₄: 475.3, found 475.3

### 3-(3-(2-(6-hydroxy-3-phenyl-3a-(1-phenylvinyl)-1,3a,4,5,6,6a-hexahydropentalen-2-yl) ethoxy) propoxy) propanoic acid (18b; 10CA-Diether; 10CA-DE)

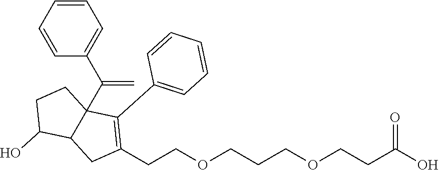 [SMILES: C=C(c1ccccc1)C12CCC(O)C1CC(CCOCCCOCCC(=O)O)=C2c1ccccc1]

Compound 14b (125 mg) was dissolved in 10 mL acetonitrile and 5 drops of 12M HCl. The reaction was stirred for 10 minutes, then concentrated and purified by prep HPLC (15-75% ACN in water, 0.1% formic acid modifier, 30 minute gradient). 97 mg, 85% 1H NMR (600 MHZ, Chloroform-d) δ 7.37 (dd, J=6.6, 2.9 Hz, 2H), 7.32 (t, J=7.3 Hz, 2H), 7.28 (d, J=7.8 Hz, 6H), 5.09 (s, 1H), 5.03 (s, 1H), 4.01-3.93 (m, 1H), 3.70 (t, J=6.2 Hz, 2H), 3.54 (t, J=6.3 Hz, 2H), 3.44 (qd, J=9.2, 4.6 Hz, 4H), 2.61 (t, J=6.1 Hz, 2H), 2.42-2.33 (m, 2H), 2.31 (dd, J=12.6, 6.9 Hz, 2H), 2.14 (d, J=17.2 Hz, 1H), 1.83 (p, J=6.3 Hz, 2H), 1.74 (tt, J=10.9, 5.5 Hz, 1H), 1.49 (pd, J=7.1, 3.4 Hz, 1H), 1.43 (q, J=7.7, 6.6 Hz, 1H), 1.36 (p, J=3.5 Hz, 1H). LRMS (APCI) m/z [M+H]⁺ calc'd for C₃₀H₃₆O₅: 477.3, found 477.3

### 5-(4-(3-phenyl-3a-(1-phenylvinyl)-6-(sulfamoylamino)-1,3a,4,5,6,6a-hexahydropentalen-2-yl) butoxy) pentanoic acid (18c; 6N-10CA-Monoether; 6N-10CA-ME)

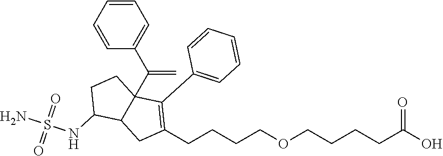 [SMILES: C=C(c1ccccc1)C12CCC(NS(N)(=O)=O)C1CC(CCCCOCCCCC(=O)O)=C2c1ccccc1]

According to the general deprotection procedure, 17a (40 mg, 0.06 mmol) was reacted with 2 drops of HCl in dioxane (1.2 mL) to give the title compound (15.2 mg, 46% yield) as a clear oil after flash chromatography (50-100% EtOAc/Hexanes). 1H NMR (600 MHZ, Chloroform-d) δ 7.34-7.27 (m, 4H), 7.26-7.24 (m, 5H), 7.19-7.15 (m, 2H), 5.55 (d, J=8.2 Hz, 1H), 5.10 (d, J=1.2 Hz, 1H), 4.91 (d, J=1.2 Hz, 1H), 4.75 (s, 2H), 3.79 (dtd, J=11.5, 8.5, 5.8 Hz, 1H), 3.51-3.36 (m, 5H), 2.63 (td, J=9.0, 2.3 Hz, 1H), 2.47 (dd, J=17.8, 2.4 Hz, 1H), 2.39 (t, J=7.5 Hz, 2H), 2.21-2.10 (m, 3H), 1.98 (dq, J=10.3, 6.3, 4.9 Hz, 2H), 1.80 (dt, J=14.8, 7.4 Hz, 1H), 1.72 (tq, J=18.0, 6.5, 5.4 Hz, 3H), 1.62 (p, J=6.7 Hz, 3H), 1.58-1.46 (m, 6H). LRMS (APCI) m/z [M+H]⁺ calc'd for C₃₁H₄₁N₂O₅S: 553.7, found 553.3

### 3-(3-(2-(3-phenyl-3a-(1-phenylvinyl)-6-(sulfamoylamino)-1,3a,4,5,6,6a-hexahydropentalen-2-yl) ethoxy) propoxy) propanoic acid (18d; 6N-10CA-Diether; 6N-10CA-DE)

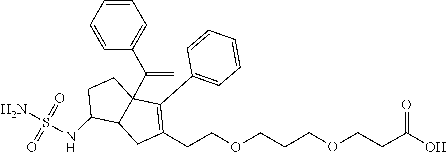 [SMILES: C=C(c1ccccc1)C12CCC(NS(N)(=O)=O)C1CC(CCOCCCOCCC(=O)O)=C2c1ccccc1]

According to the general deprotection procedure, 17b (37.5 mg, 0.05 mmol) was reacted with 2 drops of HCl in dioxane (3 mL) to give the title compound (9.6 mg, 35% yield) as a clear oil after flash chromatography (30-50% EtOAc/Hexanes). 1H NMR (600 MHZ, Chloroform-d) δ 7.40-7.34 (m, 3H), 7.30 (m, 2H), 7.25 (m, 3H), 5.47 (d, J=6.9 Hz, 2H), 5.30 (d, J=1.2 Hz, 1H), 5.08 (s, 1H), 5.07 (s, 2H), 3.83 (m, 1H), 3.71 (d, J=5.9 Hz, 2H), 3.69-3.58 (m, 2H), 3.53 (dd, J=32.9, 9.4 Hz, 5H), 2.67-2.46 (m, 6H), 1.87 (dq, J=13.4, 7.5, 6.9 Hz, 4H), 1.63 (dq, J=15.0, 8.8, 7.1 Hz, 3H), 1.40-1.31 (m, 1H). LRMS (APCI) m/z [M+H]⁺ calc'd for C₃₀H₃₉N₂O₆S: 555.7, found 555.3

### Example 2: Biological Evaluation of Novel LRH-1 Agonists and Antagonists

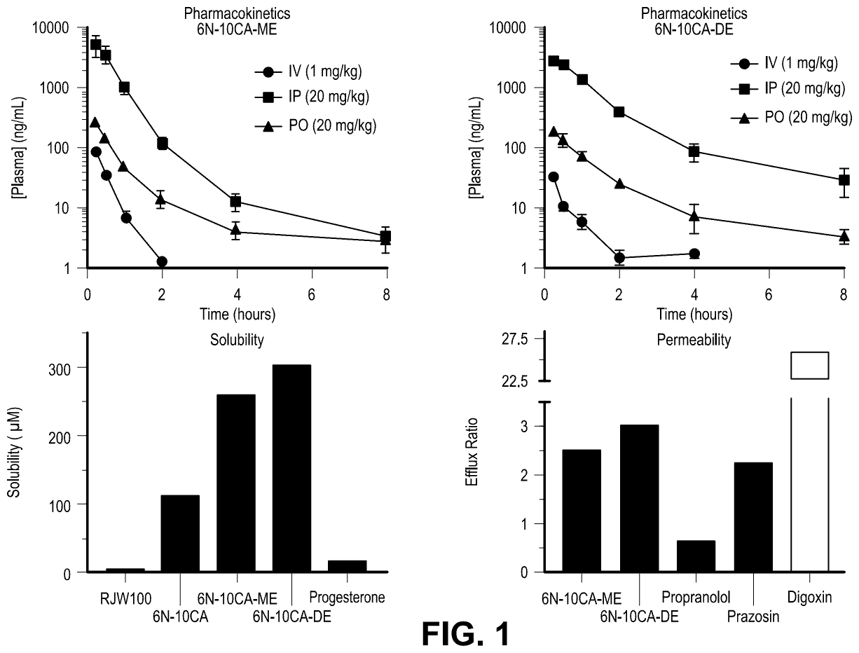

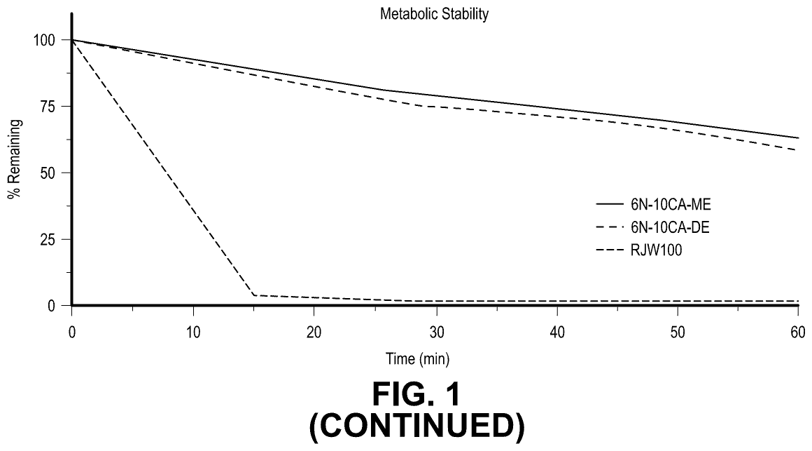

Data on pharmacokinetics, solubility, permeability, and metabolic stability of compounds disclosed herein was performed using techniques in the art. The data are provided in FIG. 1.

Protein expression and purification-LRH-1 LBD (residues 299-541) in the pMSC7 vector was expressed in BL21 (DE3) pLysS E. coli by induction with IPTG (1 mM) for 4 hr at 30° C. Protein was purified by nickel affinity chromatography. Protein used for Thermofluor™ experiments was incubated with DLPC (five-fold molar excess) for four hours at room temperature, and then repurified by size-exclusion into an assay buffer of 20 mM Tris-HCl, pH 7.5, 150 mM NaCl, and 5% glycerol. Protein used for crystallization was incubated with TEV protease to cleave the His tag. The cleaved protein was then separated from the His tag and TEV by a second round of nickel affinity chromatography. To make protein-ligand complexes, protein was incubated with ligands overnight (10-fold molar excess) and repurified by size-exclusion, using a final buffer of 100 mM ammonium acetate, pH 7.4, 150 mM sodium chloride, 1 mM DTT, 1 mM EDTA, and 2 mM CHAPS.

### Thermofluor™ Assays:

Purified LRH-1 LBD-His protein (0.2 mg/ml) was incubated overnight with 50 μM of each compound at 4° C. The final DMSO concentration in the reactions was 1%. SYPRO™ orange dye (Invitrogen; Waltham, MA) was then added at a 1:1000 dilution. Reactions were heated at a rate of 0.5° C. per minute, using a StepOne™ Plus Real Time PCR System (ThermoFisher; Waltham, MA). Fluorescence was recorded at every degree using the ROX filter (602 nm). Data were analyzed by first subtracting baseline fluorescence (ligands+SYPRO™ with no protein) and then fitting the curves using the Bolzman equation to determine the Tm. The FP competition (binding) results are shown below in Table 1.

TABLE 1

| Compound ID | Structure | Kᵢ (nM) |
|---|---|---|
| 10CA | 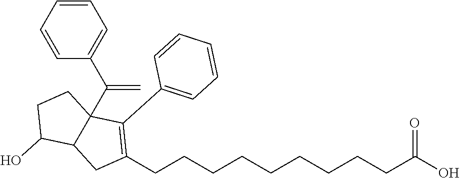 [SMILES: C=C(c1ccccc1)C12CCC(O)C1CC(CCCCCCCCCC(=O)O)=C2c1ccccc1] | 39 nM [25, 60] |
| 10CA-ME | 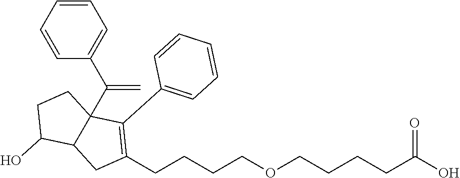 [SMILES: C=C(c1ccccc1)C12CCC(O)C1CC(CCCCOCCCCC(=O)O)=C2c1ccccc1] | 110 nM [83, 150] |
| 10CA-DE | 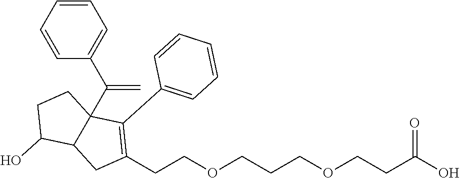 [SMILES: C=C(c1ccccc1)C12CCC(O)C1CC(CCOCCCOCCC(=O)O)=C2c1ccccc1] | 10000 nM [6100, 17000] |
| 6N-10CA | 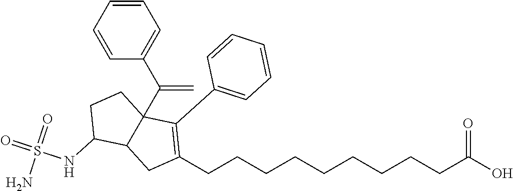 [SMILES: C=C(c1ccccc1)C12CCC(NS(N)(=O)=O)C1CC(CCCCCCCCCC(=O)O)=C2c1ccccc1] | 1.3 nM [0.96, 1.8] |
| 6N-10CA-ME | 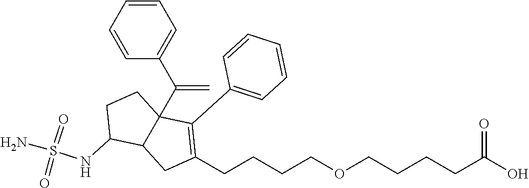 [SMILES: C=C(c1ccccc1)C12CCC(NS(N)(=O)=O)C1CC(CCCCOCCCCC(=O)O)=C2c1ccccc1] | 12 nM [5.8, 24] |
| 6N-10CA-DE | 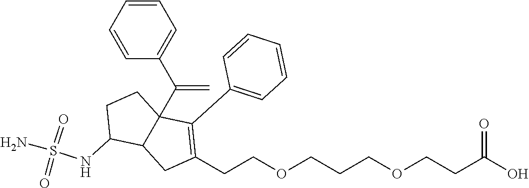 [SMILES: C=C(c1ccccc1)C12CCC(NS(N)(=O)=O)C1CC(CCOCCCOCCC(=O)O)=C2c1ccccc1] | 480 nM [270, 830] |

### Luciferase Reporter Assays:

HeLa cells were seeded at a density of 10,000 cells per well in white-walled, clear-bottomed 96-well culture plates. The next day, cells were transfected with LRH-1 and reporters, using Fugene™ HD (Roche; Indianapolis, IN) at a ratio of 5:2 Fugene™ (μl): DNA (μg). The transfected plasmids included full-length LRH-1 in a pCI vector (5 ng/well), and a SHP-luc reporter, encoding the LRH-1 response element and surrounding sequence from the SHP promoter cloned upstream of firefly luciferase in the pGL3 basic vector (50 ng/well). Cells were also co-transfected with a constitutive Renilla luciferase reporter (utilizing the CMV promoter), which was used for normalization of firefly signal (1 ng/well). Control cells received pCI empty vector at 5 ng/well in place of LRH-1-pCI. Following an overnight transfection, cells were treated with agonists for 24 hours at the concentrations indicated in the figure legends. Agonists were dissolved in DMSO and then diluted into media, with a final concentration of 0.3% DMSO in all wells. Luciferase signal was quantified using the DualGlo™ kit (Promega; Madison, WI). Experiments were conducted at least two times in triplicate. Compounds were synthesized and evaluated in biological assays. The luciferase reporter (activation assay) results are shown below in Table 2.

TABLE 2

| Compound ID | Structure | EC₅₀ (nM) |
|---|---|---|
| 10CA | 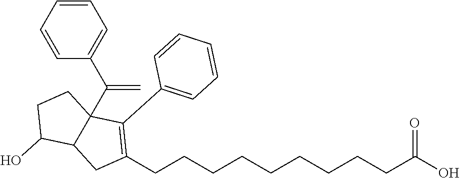 [SMILES: C=C(c1ccccc1)C12CCC(O)C1CC(CCCCCCCCCC(=O)O)=C2c1ccccc1] | 1100 nM [770, 1700] |
| 10CA-ME | 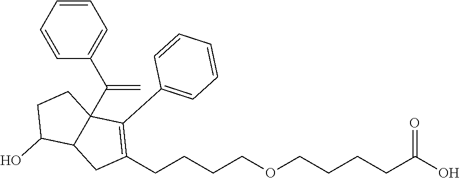 [SMILES: C=C(c1ccccc1)C12CCC(O)C1CC(CCCCOCCCCC(=O)O)=C2c1ccccc1] | 870 nM [420, 1800] |
| 10CA-DE | 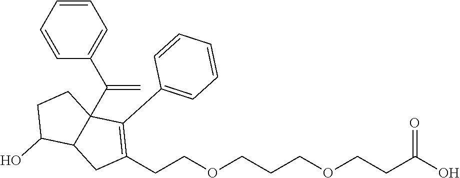 [SMILES: C=C(c1ccccc1)C12CCC(O)C1CC(CCOCCCOCCC(=O)O)=C2c1ccccc1] | 3200 nM [1700, 6300] |
| 6N-10CA | 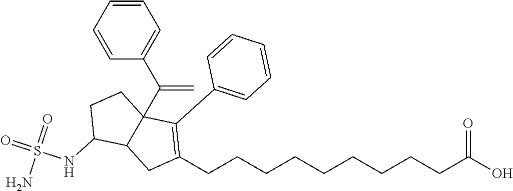 [SMILES: C=C(c1ccccc1)C12CCC(NS(N)(=O)=O)C1CC(CCCCCCCCCC(=O)O)=C2c1ccccc1] | 63 nM [31, 130] |
| 6N-10CA-ME | 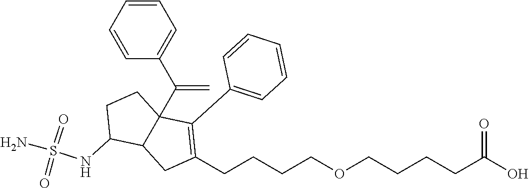 [SMILES: C=C(c1ccccc1)C12CCC(NS(N)(=O)=O)C1CC(CCCCOCCCCC(=O)O)=C2c1ccccc1] | 50 nM [19, 130] |
| 6N-10CA-DE | 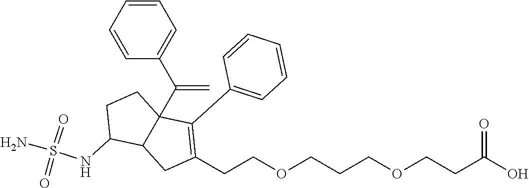 [SMILES: C=C(c1ccccc1)C12CCC(NS(N)(=O)=O)C1CC(CCOCCCOCCC(=O)O)=C2c1ccccc1] | 1200 nM [540, 2700] |

### Thermal Stability Assays:

Thermal shift was also assessed for each of the compounds to determine the protein thermal stability. Thermal stability of the LRH-1 LBD complexed with ligands was determined using a Tycho NT.6 Nanotemper. The LRH-1 LBD was incubated with 5-fold molar excess of ligand (final DMSO concentration was 1.4%) overnight at 4° C. in assay buffer (150 mM NaCl, 20 mM Tris-HCl, and 5% glycerol (pH 7.4)). Complexes were centrifuged at high speed for five minutes and then loaded into capillaries. Tryptophan/tyrosine fluorescence was monitored at wavelengths of 330 nm and 350 nm over a 30° C./min gradient (35° C.-95° C.). The inflection point was determined with Tycho NT.6 software. Two separate experiments were conducted in triplicate. Calculated inflection point values were displayed as bar graphs constructed with Prism (version 9). The inflection point (melting point) for each of the compounds is shown below in Table 3.

TABLE 3

| Compound | | Inflection Point |
|---|---|---|
| ID | Structure | (Thermal Stability) |
| 10CA | 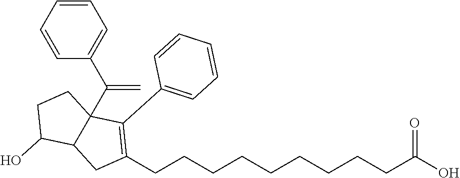 [SMILES: C=C(c1ccccc1)C12CCC(O)C1CC(CCCCCCCCCC(=O)O)=C2c1ccccc1] | 61° C. (+5.9° C.) |
| 10CA-ME | 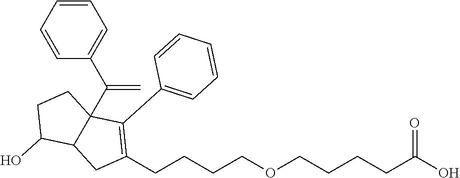 [SMILES: C=C(c1ccccc1)C12CCC(O)C1CC(CCCCOCCCCC(=O)O)=C2c1ccccc1] | 59° C. (+3.9° C.) |
| 10CA-DE | 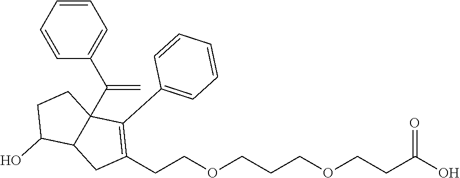 [SMILES: C=C(c1ccccc1)C12CCC(O)C1CC(CCOCCCOCCC(=O)O)=C2c1ccccc1] | 57° C. (+1.9° C.) |
| 6N-10CA | 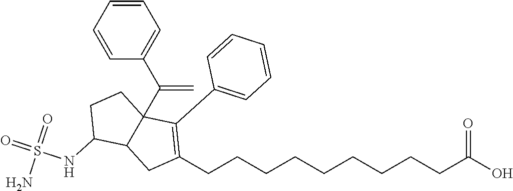 [SMILES: C=C(c1ccccc1)C12CCC(NS(N)(=O)=O)C1CC(CCCCCCCCCC(=O)O)=C2c1ccccc1] | 67° C. (+11.9° C.) |
| 6N-10CA-ME | 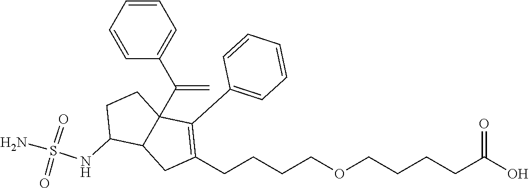 [SMILES: C=C(c1ccccc1)C12CCC(NS(N)(=O)=O)C1CC(CCCCOCCCCC(=O)O)=C2c1ccccc1] | 65° C. (+9.9° C.) |
| 6N-10CA-DE | 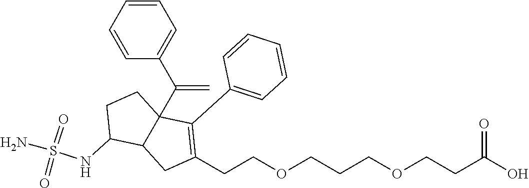 [SMILES: C=C(c1ccccc1)C12CCC(NS(N)(=O)=O)C1CC(CCOCCCOCCC(=O)O)=C2c1ccccc1] | 61° C. (+5.9° C.) |

### Example 3: Compound Distribution and Target

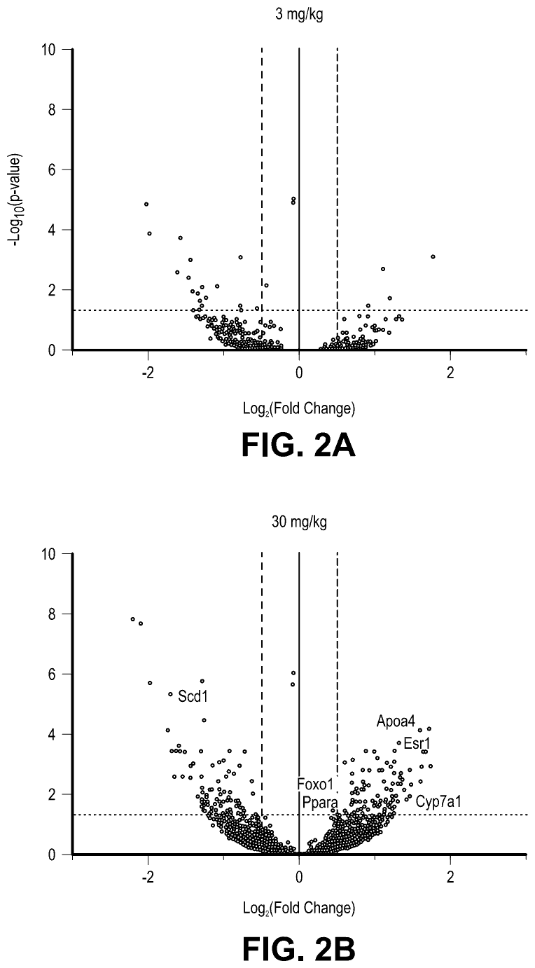

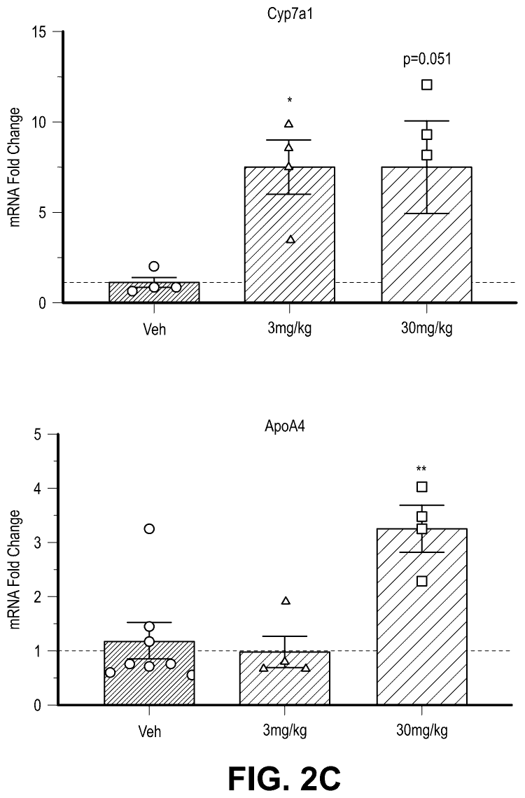

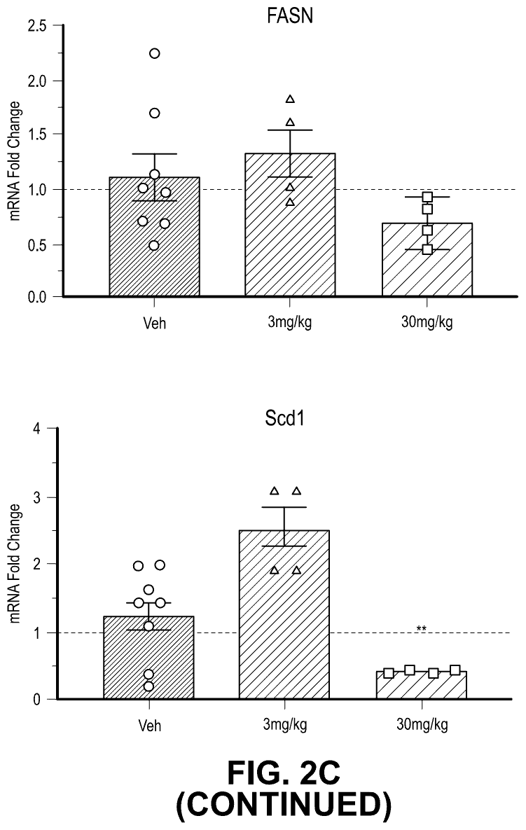

The tissue distribution of compounds as described herein and the dosage needed for the in vivo activation of hepatic LRH-1 were evaluated. For these studies, H-mLRH-1 mice (male and female; 10 weeks of age) were used. A single dose time course experiment was conducted in which different cohorts of H-mLRH-1 mice (n=6) were given a high dose (i.e., 30 mg/kg compound) via oral and intraperitoneal (IP) delivery. Liver tissue was harvested and the gene expression of Cyp7a1, Scarb1, ApoA4, and Shp was measured using standard qPCR methods at 1, 3, 6, 12, and 24 hrs. The time of peak target gene expression for a dose finding study was selected. Different cohorts of H-mLRH-1 mice were given 6 doses of the compounds using a half-log scale (i.e., 0, 0.003, 0.03, 0.3, 3, and 30 mg/kg) via oral or IP delivery. Collected liver tissue was used to monitor the expression of the genes listed above. 6N-10CA monoether showed desired gene expression activity such as elevation of Cyp7a1 and Apoa4 and a reduction in Fasn in addition to novel gene targets at 30 mg/Kg IP injection. The results are shown in FIGS. 2A, 2B, and 2C.

### Example 4: Assessment of Off-Target Effects of Compounds

6N-10CA-ME displays SF-1 activity as measured by LUC-reporter gene activation cultured cells. To evaluate if 6N-10CA monoether activates SF-1 in vivo at the optimal dose determined to drive hepatic LRH-1, the activation of SF-1 was evaluated. Specifically, adrenal glands and gonadal tissue were collected from all mice sacrificed.

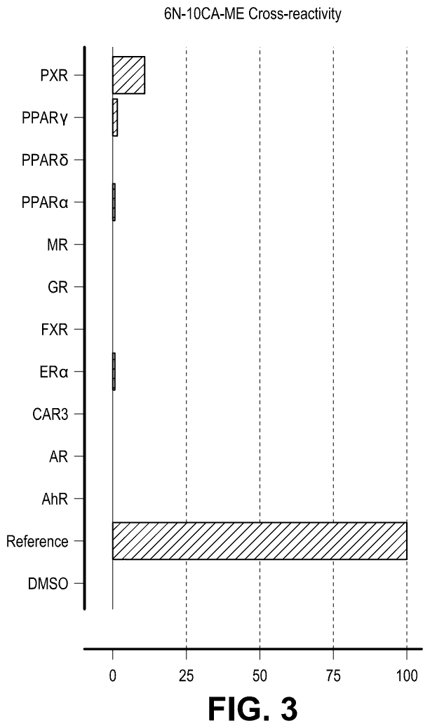

SF-1 activity was assessed using RT-qPCR for target genes (e.g., Star, Fdps, Hmgcr), though morphological/histological examination of these organs and by monitoring circulating levels of cortisol, aldosterone and androsterone using targeted LC/MS/MS. Preliminary data show minimal off-target NR activation (FIG. 3).

### Example 5: Efficacy of LRH-1 Modulators as Anti-Diabetic Agents in Obesity

The activation of LRH-1 was shown to attenuate hepatic steatosis and improve glucose homeostasis in experimental models of diet-induced obesity (DIO) and insulin resistance. In this study, a mouse model of DIO was used to evaluate the in vivo efficacy of the compounds as described herein, including the ability of the compounds to (1) attenuate hepatic steatosis and (2) improve glucose tolerance and insulin resistance.

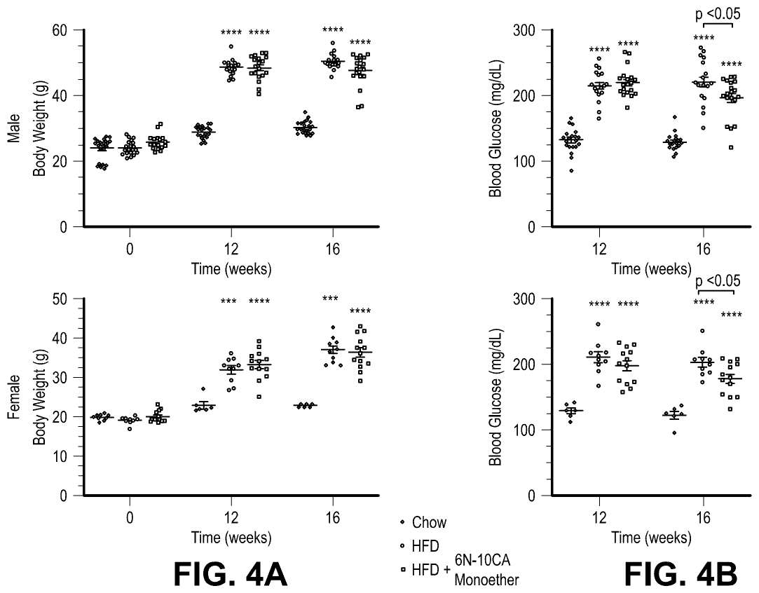

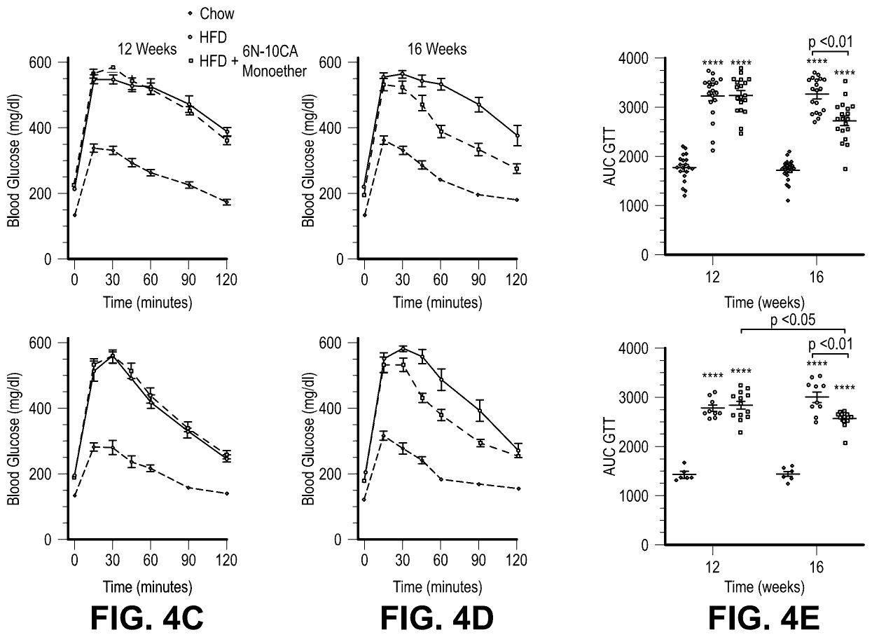

H-mLRH-1 mice (male and female) were fed one of the following diets (OpenSource Diets) for 16 weeks starting at 8 weeks of age: (1) Control Diet (10% fat diet, D12450Ji) or (2) High Fat Diet (HFD, 60% fat; D12492i). Compounds and vehicles were given to cohorts of mice starting at 12 weeks of HFD feeding, which targets the metabolic perturbations associated with HFD rather than preventing them. The compounds tested to date do not activate (or repress) mouse LRH-1. Data from male and female mice treated with 3 mg/kg 6N-10CA monoether are shown in FIG. 4.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A compound having the following formula: 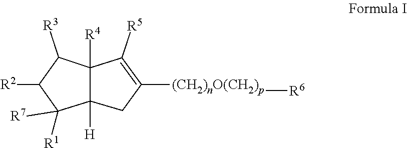
or a prodrug, salt, or stereoisomer thereof, wherein:
n is 1-6;
p is 1-6;
R¹ is alkyl, halogen, nitro, cyano, hydroxy, amino, sulfamoylamino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is a hydrogen, carboxy, or alkyl wherein R⁶ is optionally terminally substituted with a hydroxy, carboxy, or phosphate, wherein the hydroxy, carboxy, or phosphate are optionally further substituted with R¹⁰;
R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁷ is optionally substituted with one or more, the same or different, R¹⁰; or
R¹ and R⁷ together are an oxo or oxime, wherein the oxime is optionally substituted with one or more, the same or different, R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

2. The compound of claim 1, wherein R⁴ is 1-phenylvinyl.

3. The compound of claim 1, wherein n is 4 and p is 4.

4. The compound of claim 1, wherein R¹ is hydroxy or sulfamoylamino.

5. The compound of claim 1, wherein R¹ is sulfamoylamino, R⁶ is carboxy, and R⁷ is hydrogen.

6. A compound having the formula according to Structure I-A, Structure I-B, Structure I-C, Structure I-D, Structure I-E, or Structure I-F, or a prodrug, salt, or stereoisomer thereof,
wherein Structure I-A has the following formula: 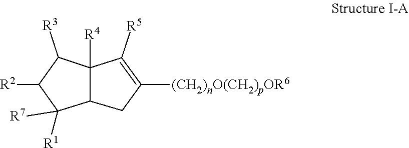
wherein:
n is 1-6;
p is 1-6;
R¹ is alkyl, halogen, nitro, cyano, hydroxy, amino, sulfamoylamino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is hydrogen, carboxy, or alkyl, wherein R⁶ is optionally substituted with an alkyl, hydroxy, carboxy, or phosphate, wherein the hydroxy, carboxy, or phosphate are optionally further substituted with R¹⁰;
R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁷ is optionally substituted with one or more, the same or different, R¹⁰; or
R¹ and R⁷ together are an oxo or oxime, wherein the oxime is optionally substituted with one or more, the same or different, R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl;
wherein Structure I-B has the following formula: 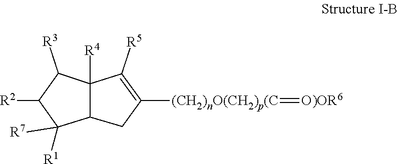
wherein:
n is 1-6;
p is 1-6;
R¹ is alkyl, halogen, nitro, cyano, hydroxy, amino, sulfamoylamino, mercapto, formyl carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is hydrogen or alkyl, wherein R⁶ is optionally terminally substituted with a hydroxy, carboxy, or phosphate, wherein the hydroxy, carboxy, or phosphate are optionally further substituted with R¹⁰; R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein Ris optionally substituted with one or more, the same or different, R¹⁰, or R¹ and R⁷ together are an oxo or oxime, wherein the oxime is optionally substituted with one or more, the same or different, R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹, and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl;
wherein Structure I-C has the following formula: 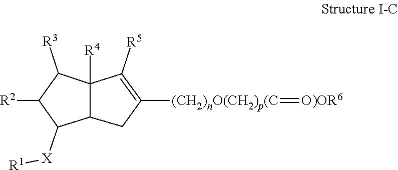
wherein:
n is 1-6;
p is 1-6;
X is a linking group, —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—
R¹ is hydrogen, hydroxy, alkyl, alkanoyl, amino, aminoalkyl, sulfamoylamino, carbamoyl, sulfate, sulfonate, aminosulfonyl, phosphate, phosphonate, or heterocyclyl;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is hydrogen or alkyl, wherein R⁶ is optionally terminally substituted with a hydroxy, carboxy, or phosphate, wherein the hydroxy, carboxy, or phosphate are optionally further substituted with R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl;
wherein Structure I-D has the following formula: 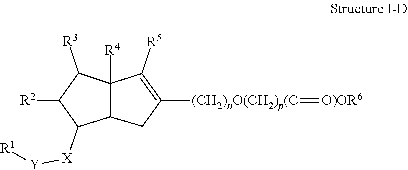
or a prodrug, salt, or stereoisomer thereof, wherein:
n is 1-6;
p is 1-6;
X is a linking group, —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—;
Y is —CH₂—, —C(OH) (OH)—, —C(OH) H, —C(Hal) (Hal)-, —C(Hal) H—, —O—, —S—, —(S—O)—, —SO₂, —NH—, —(C═O)—, —(C═NH)—, or —(C═S)—;
R¹ is hydrogen, hydroxy, alkyl, alkanoyl, amino, aminoalkyl, sulfamoylamino, carbamoyl, sulfate, sulfonate, aminosulfonyl, phosphate, phosphonate, or heterocyclyl;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is hydrogen or alkyl wherein R⁶ is optionally terminally substituted with a hydroxy, carboxy, or phosphate, wherein the hydroxy, carboxy, or phosphate are optionally further substituted with R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl;
wherein Structure I-E has the following formula: 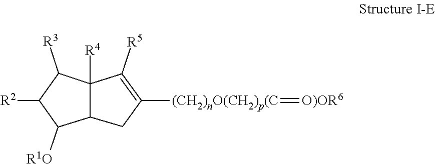
wherein:
n is 1-6;
p is 1-6;
R¹ is hydrogen, alkyl, halogen, nitro, cyano, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is a lipid or alkyl wherein R⁶ is optionally terminally substituted with a hydroxy, carboxy, or phosphate, wherein the hydroxy, carboxy, or phosphate are optionally further substituted with R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl; and
wherein Structure I-F has the following formula: 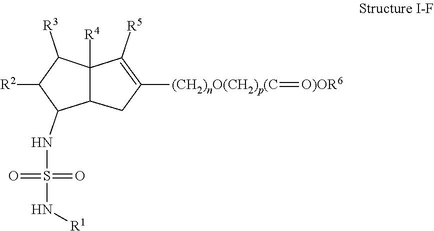
wherein:
n is 1-6;
p is 1-6;
R¹ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is a lipid or alkyl wherein R⁶ is optionally terminally substituted with a hydroxy, carboxy, or phosphate, wherein the hydroxy, carboxy, or phosphate are optionally further substituted with R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

7. The compound of claim 6, wherein R⁴ is 1-phenylvinyl.

8. The compound of claim 6, wherein n is 4 and p is 4.

9. The compound of claim 6, wherein R¹ is hydroxy or sulfamoylamino.

10. The compound of claim 6, wherein R¹ is sulfamoylamino, R⁶ is carboxy, and R⁷ is hydrogen.

11-29. (canceled)

30. A compound having the following formula: 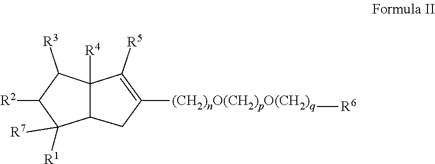
or a prodrug, salt, or stereoisomer thereof, wherein:
n is 1-6;
p is 1-6;
q is 1-6;
R¹ is alkyl, halogen, nitro, cyano, hydroxy, amino, sulfamoylamino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is a hydrogen, carboxy, or alkyl wherein R⁶ is optionally terminally substituted with a hydroxy, carboxy, or phosphate, wherein the hydroxy, carboxy, or phosphate are optionally further substituted with R¹⁰;
R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁷ is optionally substituted with one or more, the same or different, R¹⁰; or
R¹ and R⁷ together are an oxo or oxime, wherein the oxime is optionally substituted with one or more, the same or different, R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

31. The compound of claim 30, wherein n is 2, p is 3, and q is 2.

32. The compound of claim 30, wherein R⁴ is 1-phenylvinyl.

33. The compound of claim 30, R¹ is hydroxy or sulfamoylamino.

34. The compound of claim 30, wherein R¹ is sulfamoylamino and R⁷ is hydrogen.

35. The compound of claim 30, wherein R¹ is sulfamoylamino, R⁶ is carboxy, and R⁷ is hydrogen.

36. A compound having the formula according to Structure II-A, Structure II-B, Structure II-C, Structure II-D, Structure II-E, or Structure II-F, or a prodrug, salt, or stereoisomer thereof,
wherein Structure II-A has the following formula: 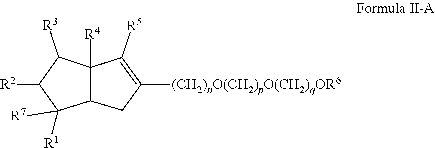
wherein:
n is 1-6;
p is 1-6;
q is 1-6;
R¹ is alkyl, halogen, nitro, cyano, hydroxy, amino, sulfamoylamino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is hydrogen, carboxy, or alkyl, wherein R⁶ is optionally substituted with an alkyl, hydroxy, carboxy, or phosphate, wherein the hydroxy, carboxy, or phosphate are optionally further substituted with R¹⁰;
R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁷ is optionally substituted with one or more, the same or different, R¹⁰; or
R¹ and R⁷ together are an oxo or oxime, wherein the oxime is optionally substituted with one or more, the same or different, R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl;
wherein Structure II-B has the following formula: 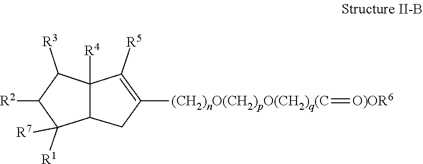
wherein:
n is 1-6;
p is 1-6;
q is 1-6;
R¹ is alkyl, halogen, nitro, cyano, hydroxy, amino, sulfamoylamino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is a hydrogen or alkyl wherein R⁶ is optionally terminally substituted with a hydroxy, carboxy, or phosphate, wherein the hydroxy, carboxy, or phosphate are optionally further substituted with R¹⁰;
R⁷ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁷ is optionally substituted with one or more, the same or different, R¹⁰; or
R¹ and R⁷ together are an oxo or oxime, wherein the oxime is optionally substituted with one or more, the same or different, R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl;
wherein Structure II-C has the following formula: 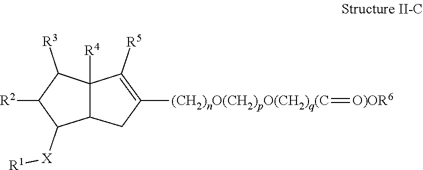
wherein:
n is 1-6;
p is 1-6;
q is 1-6;
X is a linking group, —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—
R¹ is hydrogen, hydroxy, alkyl, alkanoyl, amino, aminoalkyl, sulfamoylamino, carbamoyl, sulfate, sulfonate, aminosulfonyl, phosphate, phosphonate, or heterocyclyl;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is hydrogen or alkyl, wherein R⁶ is optionally terminally substituted with a hydroxy, carboxy, or phosphate, wherein the hydroxy, carboxy, or phosphate are optionally further substituted with R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl)₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl;
wherein Structure II-D has the following formula: 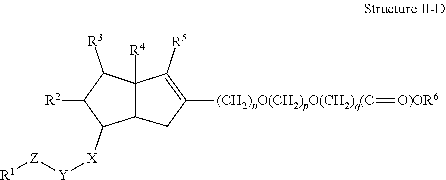
wherein:
n is 1-6;
p is 1-6;
q is 1-6;
X is a linking group, —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—;
Y is —CH₂—, —C(OH) (OH)—, —C(OH) H, —C(Hal) (Hal)-, —C(Hal) H—, —O—, —S—, —(S—O)—, —SO₂—, —NH—, —(C═O)—, —(C═NH)—, or —(C═S)—;
Z is —CH₂—, —C(OH) (OH)—, —C(OH) H, —C(Hal) (Hal)-, —C(Hal) H—, —O—, —S—, —(S═O)—, —SO₂—, —NH—, —(C═O)—, —(C═NH)—, or —(C═S)—;
R¹ is hydrogen, hydroxy, alkyl, alkanoyl, amino, aminoalkyl, sulfamoylamino, carbamoyl, sulfate, sulfonate, aminosulfonyl, phosphate, phosphonate, or heterocyclyl;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is a hydrogen or alkyl wherein R⁶ is optionally terminally substituted with a hydroxy, carboxy, or phosphate, wherein the hydroxy, carboxy, or phosphate are optionally further substituted with R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl;
wherein Structure II-E has the following formula: 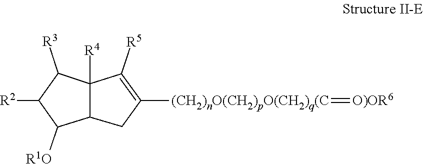
wherein:
n is 1-6;
p is 1-6;
q is 1-6;
R¹ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is a lipid or alkyl wherein R⁶ is optionally terminally substituted with a hydroxy, carboxy, or phosphate, wherein the hydroxy, carboxy, or phosphate are optionally further substituted with R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl; and
wherein Structure II-F has the following formula: 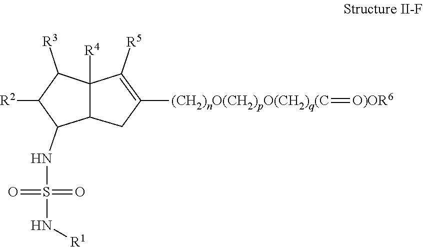
or a prodrug, salt, or stereoisomer thereof, wherein:
n is 1-6;
p is 1-6;
q is 1-6;
R¹ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R¹ is optionally substituted with one or more, the same or different, R¹⁰;
R² is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R² is optionally substituted with one or more, the same or different, R¹⁰;
R³ is hydrogen, alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R³ is optionally substituted with one or more, the same or different, R¹⁰;
R⁴ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁴ is optionally substituted with one or more, the same or different, R¹⁰;
R⁵ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, carbocyclyl, benzoyl, benzyl, aryl, or heterocyclyl, wherein R⁵ is optionally substituted with one or more, the same or different, R¹⁰;
R⁶ is a lipid or alkyl wherein R⁶ is optionally terminally substituted with a hydroxy, carboxy, or phosphate, wherein the hydroxy, carboxy, or phosphate are optionally further substituted with R¹⁰;
R¹⁰ is alkyl, halogen, nitro, cyano, hydroxy, amino, mercapto, formyl, carboxy, carbamoyl, alkoxy, hydroxyalkyl, alkylthio, thioalkyl, alkylamino, aminoalkyl, (alkyl) ₂amino, alkanoyl, alkoxycarbonyl, alkylsulfinyl, alkylsulfonyl, arylsulfonyl, sulfamoyl, aminosulfonyl, carbocyclyl, benzoyl, benzyl, aryl, or heterocyclyl, wherein R¹⁰ is optionally substituted with one or more, the same or different, R¹¹; and
R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, isopropoxy, tert-butoxy, hydroxymethyl, hydroxyethyl, thiomethyl, thioethyl, aminomethyl, aminoethyl, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, isopropoxycarbonyl, tert-butoxycarbonyl, N-methylsulfamoyl, N-ethylsulfamoyl, N,N-dimethylsulfamoyl, N,N-diethylsulfamoyl, N-methyl-N-ethylsulfamoyl, benzoyl, benzyl, carbocyclyl, aryl, or heterocyclyl.

37. The compound of claim 36, wherein n is 2, p is 3, and q is 2.

38. The compound of claim 36, wherein R⁴ is 1-phenylvinyl.

39. The compound of claim 36, R¹ is hydroxy or sulfamoylamino.

40. The compound of claim 36, wherein R¹ is sulfamoylamino and R⁷ is hydrogen.

41. The compound of claim 36, wherein R¹ is sulfamoylamino, R⁶ is carboxy, and R⁷ is hydrogen.

42-61. (canceled)

62. A compound of the following structure: 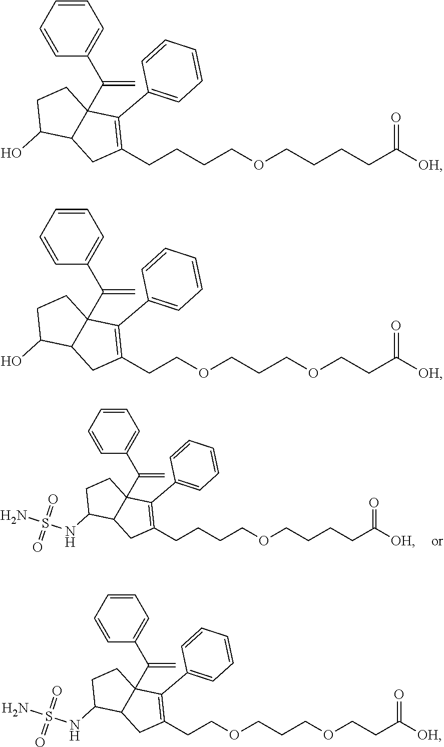 [SMILES: C=C(c1ccccc1)C12CCC(O)C1CC(CCCCOCCCCC(=O)O)=C2c1ccccc1 ; C=C(c1ccccc1)C12CCC(O)C1CC(CCOCCCOCCC(=O)O)=C2c1ccccc1 ; C=C(c1ccccc1)C12CCC(NS(N)(=O)=O)C1CC(CCCCOCCCCC(=O)O)=C2c1ccccc1 ; C=C(c1ccccc1)C12CCC(NS(N)(=O)=O)C1CC(CCOCCCOCCC(=O)O)=C2c1ccccc1]
or a prodrug or salt thereof.

63-65. (canceled)

66. A pharmaceutical composition comprising a compound as in claim 1 or a pharmaceutically acceptable salt and a pharmaceutically acceptable excipient.

67. A method of treating cancer, diabetes, cardiovascular disease, inflammatory bowel diseases (IBD), colitis or ulcerative colitis, or diabetic nephropathy, comprising administering an effective amount of a pharmaceutical composition of claim 66 to a subject in need thereof.

68. The method of claim 67, wherein the cancer is bladder cancer, breast cancer, colon cancer, rectal cancer, endometrial cancer, kidney cancer, leukemia, lung cancer, melanoma, non-Hodgkin lymphoma, pancreatic cancer, prostate cancer, or thyroid cancer, or wherein the diabetes is insulin-dependent diabetes mellitus, non insulin-dependent diabetes mellitus, or gestational diabetes.

69-74. (canceled)
